(19) United States
(12) Patent Application Publication
(10) Pub. No.: US 2023/0210897 A1
(43) Pub. Date: Jul. 6, 2023

(54) MODIFIED NATURAL KILLER CELLS AND METHODS OF USING THE SAME

(71) Applicants: CHILDREN’S NATIONAL MEDICAL CENTER, Washington, DC (US); THE GEORGE WASHINGTON UNIVERSITY, Washington, DC (US)

(72) Inventors: Catherine Mary BOLLARD, Bethesda, MD (US); Conrad Russell Y. CRUZ, Bethesda, MD (US); Rachel A. BURGA, Washington, DC (US); Eric YVON, Washington, DC (US)

(73) Assignees: CHILDREN’S NATIONAL MEDICAL CENTER, Washington, DC (US); THE GEORGE WASHINGTON UNIVERSITY, Washington, DC (US)

(21) Appl. No.: 17/626,013

(22) PCT Filed: Jul. 12, 2019
(86) PCT No.: PCT/US2019/041739

§ 371 Date: Jan. 10, 2022

(51) Int. Cl. A61K 35/17; C07K 14/71; C07K 14/55; C07K 14/54; C07K 14/435; C07K 14/705; A61P 35/00; C07K 14/725; A61P 25/00

(52) U.S. Cl. CPC A61K 35/17; A61P 25/00; A61P 35/00; C07K 14/54; C07K 14/55; C07K 14/71; C07K 14/435; C07K 14/705; C07K 14/5434; C07K 14/5443; C07K 14/7051; C07K 14/7056; C07K 14/70503; C07K 2319/02; C07K 2319/03

(57) ABSTRACT

The disclosure provides modified NK cells and pharmaceutical compositions comrpsing the same. The disclosure also provides methods of treating cancer using the same.

### FIELD

The present invention provides natural killer (NK) cell compositions, therapies and processes of manufacture that are tailored to inactivate tumor growth factor-beta (TGFβ) secreted by a patient’s cancer cell to encourage a more immunogenic microenvironment for cancer cell clearance. The present invention also extends to methods of manufacturing such NK cell compositions to provide cancer immunotherapy.

### BACKGROUND

Natural killer (NK) cells are cytotoxic lymphocytes that constitute a major component of the innate immune system, NK cells do not express T-cell antigen receptors (TCR), CD3 or surface immunoglobulins (1 g) B cell receptor. NK cells generally express the surface markers CD16 (FoγRIII) and CD56 in humans, but a subclass of human NK cells is CD16---. NK cells are cytotoxic; small granules in their cytoplasm contain special proteins such as perforin and proteases known as granzymes. Upon release in close proximity to a cell targeted for killing, perforin forms pores in the cell membrane of the target cell through which the granzymes and associated molecules can enter, inducing apoptosis. One granzyme, granzyme B (also known as granzyme 2 and cytotoxic T-lymphocyte-associated serine esterase 1), is a serine protease crucial for rapid induction of target cell apoptosis in the cell-mediated immune response.

NK cells are activated in response to interferons or macrophage-derived cytokines. Activated NK cells are referred to as lymphokine activated killer (LAK) cells. NK cells possess two types of surface receptors, labeled “activating receptors” and “inhibitory receptors,” that control the cells’ cytotoxic activity.

Among other activities, NK cells play a role in the host rejection of tumors. Because cancer cells have reduced or no class I MHC expression, they can become targets of NK cells. Accumulating clinical data suggest that haploidentical transplantation of human NK cells isolated from peripheral blood monomuclear cells (PBMC) or bone marrow mediate potent anti-leukemia effects without incurring detectable graft versus host disease (GVHD). See Ruggeri et al., Science 295:2097-2100 (2002)). Natural killer cells can become activated by cells lacking, or displaying reduced levels of, major histocompatibility complex (MHC) proteins. Additionally, the activating receptors expressed on NK cells are known to mediate detection of “stressed” or transformed cells with express ligands to activating receptors and therefore trigger the NK cell activation. For instance, NCR1 (NKp46) binds viral hemagglutinins. NKG2D ligands include CMV UL 16-binding protein 1 (ULB1), ULB2, ULB3 and MHC-class-I-polypeptide-related sequence A (MICA) and MICB proteins. NK protein 2B4 binds CD48, and DNAM-1 binds Poliovirus receptor (PVR) and Nectin-2, both are consistently detected in acute myeloid leukemia (AML). See Penda et al., Blood 105: 2066-2073 (2004). Moreover, lysis of AML has been described to be mainly natural cytotoxicity receptor (NCR) dependent. See Fauriat et al., Blood 109: 323-330 (2007). Activated and expanded NK cells and LAK cells from peripheral blood have been used in both ex vivo therapy and in vivo treatment of patients having advanced cancer, with some success against bone marrow related diseases, such as leukemia; breast cancer; and certain types of lymphoma LAK cell treatment requires that the patient first receive IL-2, followed by leukopheresis and then an ex vivo incubation and culture of the harvested autologous blood cells in the presence of IL-2 for a few days. The LAK cells must be reinfused along with relatively high doses of IL-2 to complete the therapy. This purging treatment is expensive and can cause serious side effects. These include fluid retention, pulmonary edema, drop in blood pressure, and high fever.

During tumor progression, tumor cells develop several mechanisms to either escape from NK-cell recognition and attack or to induce defective NK cells These include losing expression of adhesion molecules, costimulatory ligands or ligands for activating receptors, upregulating MHC class 1, soluble MIC, FasL or NO expression, secreting immunosuppressive factors such as IL-10, TGF-β and indoleamine 2,3-dioxygense (IDO) and resisting Fas- or perforin-mediated apoptosis Waldhauer 1, Steinle A. “NK cells and cancer immunosurveillance”. Oncogene 2008; 27: 5932-5943; Maki G, Krystal G, Dougherty G, Takei F, Klingemann HG, “Induction of sensitivity to NK-mediated cytotoxicity by TNF-alpha treatment: possible role of ICAM-3 and CD44”. Leukemia 1998; 12: 1565-1572; Costello RT, Sivori S, Marcenaro E, Lafage-Pochitaloff M, Mozziconacci MJ, Reviron D et al. « Defective expression and function of natural killer cell-triggering receptors in patients with acute myeloid leukemia”. Blood 2002; 99: 3661-3667). Whether enhanced cytotoxicity occurred due to an increase in expression of NK cell activating receptors or was the consequence of expanded NK cells having increased levels of molecules that induce tumor aptotosis (ie., TRAIL, FasL, granzymes, etc) is unclear (Childs RW, Berg M, 2013, “Bringing natural killer cells to the clinic: ex vivo manipulation”, Hematology Am Soc Hematol Educ Program.; 2013:234-46 ).

In cancer patients, NK-cell abnormalities have been observed, including decreased cytotoxicity, defective expression of activating receptors or intracellular signaling molecules, overexpression of inhibitory receptors, defective proliferation, decreased numbers in peripheral blood and in tumor infiltrate, and defective cytokine production (Sutlu T, Alici E. “Natural killer cell-based immunotherapy in cancer: current insights and future prospects”. J Intern Med 2009; 266: 154-181). Given that NK cells play critical roles in the first-line of defense against malignancies by direct and indirect mechanisms, the therapeutic use of NK. cells in human cancer immunotherapy has been proposed and followed in a clinical context.

Several strategies have been used to enhance NK-cell responses to tumors. Cytokines are used in the treatment of some human cancers and NK-cell differentiation and activation is affected by cytokines such as interleukins (e.g. IL-2, IL-12. IL-15, IL-18 and IL-21). The effect of IL-2 administration on activation and expansion of NK cells in cancer patients has been assessed in several trials, with mixed outcomes depending on the type of tumor and the conditions used for IL-2 administration. Further, such therapies involving administration of cytokines are associated with potential toxicities.

Currently, some of the most promising approaches for targeting NK cells involves adoptive cell transfer, including the use of autologous NK cells, allogeneic NK cells, NK cell lines and CAR NK cells. However, these approaches are associated with significant drawbacks, such as low efficacy, the requirement for substantial depletion of T cells to avoid GVHD (for allogeneic cells), low persistence in subjects, and difficulties in expanding and/or manufacturing large numbers of cells. Unfortunately, many solid tumors have an innate ability to evade immune surveillance by producing immunosuppressive cytokines such as transforming growth factor beta (TGFβ) which can prevent successful anti-tumor effects of cell therapie s.

Thus, there is a need in the art for alternative ways to exploit immune killer cells (e.g. NK cells and CD8+ T cells) for therapeutic purposes.

### SUMMARY OF EMBODIMENTS

The present disclosure provides a novel and inventive platform for cancer therapy that simultaneously endows immune cells with a means to resist the immunosuppressive environment as well as facilitate their own activation. Natural killer (NK) cell therapy represents a promising therapeutic platform because NK cells rapidly lyse their target cells without the need for prior exposure However, success is limited in solid tumors, such as neuroblastoma, which are frequently observed to downregulate MHC, thus preventing tumor killing by allogeneic NK cells (missing self theory). Umbilical cord blood is a promising source for allogeneic “off the shelf” NK cells, which are readily available. However, anti-tumor efficacy is limited by immunosuppressive cytokines present in the tumor microenvironment, such as TGFβ, which impairs NK-cell phenotype and function, and may therefore limit therapeutic efficacy. To overcome this limitation the present invention provides genetically-modified NK cells that express variants of a modified TGFβ receptor which couple the TGFβ dominant negative receptor to NK-specific activating domains. With this engineered receptor, TGFβ signals are effectively neutralized, and potentially converted to activating signals. These modified NK cells demonstrated higher cytotoxic activity against neuroblastoma in a TGFβ-rich environment, compared to their unmodified counterparts. The present disclosure describes the introduction of a novel and inventive feature, namely the ability to convert a suppressive signal (TGFβ) into an activating signal as a switch mechanism. As described by the present disclosure, not only will immune cells be resistant to the damaging effects of tumor-associated TGFβ, but they will also exhibit enhanced cellular activation as a direct response to TGFβ binding. This innovative approach to “hijack” the TGFβ receptor and target TGFβ in the tumor microenvironment allows for NK cells to simultaneously (1) resist the immune suppression in the microenvironment, (2) serve as cytokine sinks thereby preventing inhibition of other components of the immune response, and (3) modulate the immune environment into a more pro-immunogenic site by promoting ADCC

Accordingly, in a first aspect, the present disclosure provides a cell comprising an exogenous nucleic acid sequence comprising at least a first expressible coding sequence, the first expressible coding sequence encoding an amino acid sequence comprising a first and a second amino acid domain, wherein the first amino acid domain comprises a modified extracellular TGF-β receptor sequence capable of binding TGF- β and the second amino acid domain comprises a transmembrance or intracellular signaling sequence that is free of a biologically active modified TGF-β receptor 1 (TGF-βRI) or a modified TGF-β receptor II (TGF-βRII) intracellular domain. In some embodiments, the cell is a primary antigenic presenting cell, T-cell or NK cell from a subject. In some embodiments, the cell is a primary NK cell harvested from a subject or a cell derived from an umbilical cord blood of a subject. In some embodiments, the cell is a primary NK cell isolated from a subject or a cell derived from an umbilical cord blood of a subject. In some embodiments, the first expressible coding sequence comprises a fusion protein comprising the first amino acid domain that is free of or substantially free of a biologically active TGF-βRI or TGF-βRII intracellular domain and the second amino acid domain comprises a NK cell activation domain or sequence. In some embodiments, the first expressible coding sequence comprises a third amino acid domain encoded by a nucleic acid seqeunce comprising at least about 70%, 75%, 80%, 85%, 90%, 91%, 92%, 93%, 94%, 95%, 96%, 97%, 98%, 99%, or 100% sequence identity to SEQ ID NO:2 In some embodiments, the first expressible coding sequence comprises at least about 70%, 75%, 80%, 85%, 90%, 91%, 92%, 93%, 94%, 95%, 96%, 97%, 98%, 99%, or 100% sequence identity to SEQ ID NO:9. In some embodiments, the second amino acid domain comprises a aminon acid sequence encoded by a nucleic acid seqeunce comprising at least about 70%, 75%, 80%, 85%, 90%, 91%, 92%, 93%, 94%, 95%, 96%, 97%, 98%, 99%, or 100% sequence identity to SEQ ID NO:12, 17, 18, 19, 20, 21, 22, 23, 24, or 25.

In some embodiments, the exogenous nucleic acid sequence comprises a nucleic acid sequence comprising at least about 70%, 75%, 80%, 85%, 90%, 91%, 92%, 93%, 94%, 95%, 96%, 97%, 98%, 99%, or 100% sequence identity to SEQ ID NO:13, SEQ ID NO:14 and/or SEQ ID NO:15. In some embodiments, the exogenous nucleic acid sequence comprises a nucleic acid sequence comprising no more than about 70%, 75%, 80%, 85%, 90%, 91%, 92%, 93%, 94%, 95%, 96%, 97%, 98%, 99% sequence identity to SEQ ID NO:13, SEQ ID NO:14 and/or SEQ ID NO:15. In some embodiments, the exogenous nucleic acid sequence comprises at least about 70%, 75%, 80%, 85%, 90%, 91%, 92%, 93%, 94%, 95%, 96%, 97%, 98%, 99%, or 100% sequence identity to SEQ ID NO:13, SEQ ID NO:14 and/or SEQ ID NO: 16. In some embodiments, the expressible coding sequence further comprises at least one nucleic acid sequence that encodes a nuclear localization sequence and/or a leader sequence. In some embodiments, the at least one nucleic acid seqeuce that encodes a nuclear localization seqeunce comprises about 70%, 75%, 80%, 85%, 90%, 91%, 92%, 93%, 94%, 95%, 96%, 97%, 98%, 99%, or 100% sequence identity to SEQ ID NO:3 positioned 5′ upstream from the nucleic acid sequence encoding the first amno acid domain. In some embodiments, the cell comprises a viral vector that comprises the exogenous nucleic acid sequence. In some embodiments, the exogenous nucleic acid sequence comprises an intracellular signaling sequence capable of activating NK-cell innate immunity, such as DAP12 or a functional fragment thereof. In some embodiments, the exogenous nucleic acid sequence encodes an amino acid sequence comprising a transmembrane sequence capable of activating NK-cell innate immunity, such as those sequences from Table Z, or functional fragments thereof.

In some embodiments, either: (i) the first exogenous nucleic acid sequence further comprises a second expressible coding sequence encoding one or a plurality of interleukin molecules; or (ii) the cell further comprises a second exogenous nucleic acid sequence comprising a second expressible coding seqeunce encoding one or a plurality of interleukin molecules. In some embodiments, the interleukins are chosen from one or a combination of IL-2, IL-12, IL-15, IL-18, IL-21 or IL-27.

The disclosure also relates to a cell comprising: (i) an exogenous nucleic acid sequence comprising an expressible coding seqeunce operably linked to at least one regulatory sequence, wherein the expressible coding seqeunce encodes a fusion protein comprising at least a first, a second, and a third amino acid domain; wherein the first amino acid domain comprisies a modified extracellular TGF-β receptor sequence, the second amino acid domain comprises an intracellular signaling sequence that is free of a biologically active TGF-β receptor 1 or TGF-βRII intracellular domains; and the third amino acid domain comprises at least one isolation amino acid sequence; (ii) from about 5 to about 50 copies/density of CD16 or CD19 or functional fargments thereof. In some embodiments, the first amino acid domain comprises at least 70% sequence identity to an extracellular portion of human TGFβ-RI or TGFp-RII. In some embodiments, the second amino acid domain comprises one or a combination of: human DAP-12, human KIR2DS1, KIR2DS2, human KIR2DS3, human KIR2DS4, KIR2DS5, human KIR3DS1, human NKp44, human NKG2C, human NKG2E, human NOTCH1, NOTCH2, NOTCH3, NOTCH4 or a functional fragment thereof. In some embodiments, the third amino acid domain comprises a truncated form of human CD19.

IN some embodoiments, the disclosure relates to a pharmaceutical composition comprising: (i) a therapeutically effective amount of one or a plurality cells disclosed herein; and (ii) a pharmaceutically acceptable carrier. In some embodiments, the disclosure relates to a composition comprising an isolated nucleic acid sequence: (i) comprising at least about 70%, 75%, 80%, 85%, 90%, 91%, 92%, 93%, 94%, 95%, 96%, 97%, 98%, 99%, or 100% sequence identity to SEQ ID NO:4; (ii) comprising at least about 70%, 75%, 80%, 85%, 90%, 91%, 92%, 93%, 94%, 95%, 96%, 97%, 98%, 99%, or 100% sequence identity to SEQ ID NO:4 and at least about 70%, 75%, 80%, 85%, 90%, 91%, 92%, 93%, 94%, 95%, 96%, 97%, 98%, 99%, or 100% sequence identity to SEQ ID NO:5; or (iii) comprising at least about 70%, 75%, 80%, 85%, 90%, 91%, 92%, 93%, 94%, 95%, 96%, 97%, 98%, 99%, or 100% sequence identity to or SEQ ID NO:4 and at least about 70%, 75%, 80%, 85%, 90%, 91%, 92%, 93%, 94%, 95%, 96%, 97%, 98%, 99%, or 100% sequence identity to SEQ ID NO:5; wherein the isolated nucleic acid sequence is free of a SEQ ID NO:6 or any functional fragment thereof.

In some embodiments, the disclosure relates to a composition comprising an isolated nucleic acid sequence: (i) comprising at least about 70%, 75%, 80%, 85%, 90%, 91%, 92%, 93%, 94%, 95%, 96%, 97%, 98%, 99%, or 100% sequence identity to SEQ ID NO.4; (ii) comprising at least about 70%, 75%, 80%, 85%, 90%, 91%, 92%, 93%, 94%, 95%, 96%, 97%, 98%, 99%, or 100% sequence identity to SEQ ID NO:4 and at least about 70%, 75%, 80%, 85%, 90%, 91%, 92%, 93%, 94%, 95%, 96%, 97%, 98%, 99%, or 100% sequence identity to SEQ ID NO:5; or (iii) comprising at least about 70%, 75%, 80%, 85%, 90%, 91%, 92%, 93%, 94%, 95% 96%, 97%, 98%, 99%, or 100% sequence identity to or SEQ ID NO:4 and at least about 70%, 75%, 80%, 85%, 90%, 91%, 92%, 93%, 94%, 95%, 96%, 97%, 98%, 99%, or 100% sequence identity to SEQ ID NO: 5.

In some embodiments, the isolated nucleic acid sequence further comprises: (i) at least about 70%, 75%, 80%, 85%, 90%, 91%, 92%, 93%, 94%, 95%, 96%, 97%, 98%, 99%, or 100% sequence identity to SEQ ID NO:9; or (ii) a nucleic acid sequence comprising at least about 70%, 75%, 80%, 85%, 90%, 91%, 92%, 93%, 94%, 95%, 96%, 97%, 98%, 99%, or 100 % sequence identity to SEQ ID NO:9 and a nucleic acid sequence comprising at least about 70%, 75%, 80%, 85%, 90%, 91%, 92%, 93%, 94%, 95%, 96%, 97%, 98%, 99%, or 100% sequence identity to SEQ ID NO:8. In some embodiments, the isolated nucleic acid sequence further comprises at least about 70%, 75%, 80%, 85%, 90%, 91%, 92%, 93%, 94%, 95%, 96%, 97%, 98%, 99% or 100% sequence identity to SEQ ID NO:7.

In some embodiments, the isolated nucleic acid sequence further comprises: (i) at least about 70%, 75%, 80%, 85%, 90%, 91%, 92%, 93%, 94%, 95%, 96%, 97%, 98%, 99%, or 100% sequence identity to SEQ ID NO: 12; or (ii) a nucleic acid sequence comprising at least about 70%, 75%, 80%, 85%, 90%, 9 1%, 92%, 93%, 94%, 95%, 96%, 97%, 98%, 99%, or 100% sequence identity to SEQ ID NO:12 and a nucleic acid sequence comprising at least about 70%, 75%, 80%, 85%, 90%, 91%, 92%, 93%, 94%, 95%, 96%, 97%, 98%, 99%, or 100% sequence identity to SEQ ID NO: 11.

In some embodiments, wherein the isolated nucleic acid sequence further comprises: (i) at least about 70%, 75%, 80%, 85%, 90%, 91%, 92%, 93%, 94%, 95%, 96%, 97%, 98%, 99%, or 100% sequence identity to SEQ ID NO:12; or (ii) a nucleic acid sequence comprising at least about 70%, 75%, 80%, 85%, 90%, 91%, 92%, 93%, 94%, 95%, 96%, 97%, 98%, 99%, or 100% sequence identity to SEQ ID NO: 12 and a nucleic acid sequence comprising at least about 70%, 75%, 80%, 85%, 90%, 91%, 92%, 93%, 94%, 95%, 96%, 97%, 98%, 99%, or 100% sequence identity to SEQ ID NO: 11. In some embodiments, the isolated nucleic acid sequence further comprises a nucleic acid at least about 70%, 75%, 80%, 85%, 90%, 91%, 92%, 93%, 94%, 95%, 96%, 97%, 98%, 99%, or 100% sequence identity to SEQ ID NO:3 and, when oriented from the 5′ to the 3′ oritentation, positioned 5′ upstream from SEQ ID NO:9

In some embodiments, the isolated nucleic acid sequence further comprises at least one linker between any one or more sequence identifiers. In some embodiments, the linker comprises a nucleic acid at least about 70%, 75%, 80%, 85%, 90%, 91%, 92%, 93%, 94%, 95%, 96%, 97%, 98%, 99%, or 100% sequence identity to SEQ ID NO: 1.

In some aspects, the dsiclsure relates to a composition comprising a plasmid, the plasmid comprising any one or plurality of isolated nulcleic acid sequences disclosed herein. IN some embodiments, the disclosure relates to a viral vector comprising a any one or plurality of isolated nulcleic acid sequences disclosed herein. In some embodiments, the viral vector is a nonpathogenic AAV vector, retroviral vector, or lentiviral vector.

The disclosure also relates to a cell, such as a isolated NK cell, disclosed herein comprising a plasmid comprising any one or plurality of isolated nulcleic acid sequences disclosed herein.

The disclosure relates to a pharmaceutical composition comprising: (i) a pharmaceutically effective amount of modified NK cells disclosed herein; and (ii) a pharmceutically acceptable carrier.

In some aspects, the disclosure relates to a method for inducing cell death of a target cell, the method comprising: (a) contacting a pharmaceutically effective amount of any one or plurality of cell disclosed herein to a target cell. In some embodiments, the method further comprises contacting the target cell with one or a plurality of cytokines or a nucleic acid encoding one or a plurality of cytokines. In some embodiments, the cytokines are chosen from one or a combination of IL-2, IL-12, IL-15, IL-18, IL-21 or IL-27. In some embodiments, the cells disclosed herein are further transduced with a plasmid or plurality of plasmids encoding any one or plurality of disclosed cytokines or functional fragments herein. In some embodiments, the one or plurality of cells are contacted with one or a plurality of target cells for a time period sufficient for the one or plurality of cells to secrete an amount of granzymes and/or porforin into the target cell. In some embodiments, the one or plurality of cells are contacted with one or a plurality of target cells for a time period sufficient for the one or plurality of cells to secrete an amount of granzymes and/or porforin into the target cell sufficient to kill the target cell. In some embodiments, the target cell is a cancer cell or a cancer cell within a solid tumor .In some embodiments, the target cell is a cancer cell exhibiting dysfunctional secretion of TGFβ. In some embodiments, the target cell is a brain cell or a metastatic cell derived from the brain.

A method of treating brain cancer in a subject in need thereof, the method comprising administering to the subject a therapeutically effective amount of: (i) one or a plurality of cells disclosed herein; or (ii) a pharmaceutical composition disclosed herein. In some embodiments, the brain cancer is a solid tumor . In some embodiments, the brain cancer is neuroblastoma. In some embodiments, the brain cancer is pediatric neuroblastoma characterized by one or a plurality of cells overexpressing TGFβ relative to expression of TGFβ in a non-cancerous cell of the same or similar cell type from which the cancer cell is derived. In some embodiments, the level of TGFβ expression is detected by immunohistochmestry, fluorescence of one or a plurality of probes, microarray, or PCR.

A method of treating a hyperproliferative disorder, such as a cancer, characterized by dysfunctional or increased of expression of TGFβ in a subject in need thereof, the method comprising administering to the subject a therapeutically effective amount of: (i) one or a plurality of cells disclosed herein; or (ii) a pharmaceutical composition disclosed herein.

The disclosure relates to a method of preventing progression of cancer in a subject in need thereof, the method comprising administering to the subject a therapeutically effective amount of: (i) one or a plurality of cells disclosed herein or one or a plurality of pharmaceutical compositions disclosed herein. In some aspects, the disclosure relates to a method of targeting and/or killing a hyperproliferative cell, such as a cancer cell in a subject, the method comprising administering to the subject a therapeutically effective amount of: (i) one or a plurality of cells disclosed herein; or (ii) a pharmaceutical composition comprising any of the nucleic acid molecules expressing the amino acid sequences disclosed herein.The method of any of claims 41 through 44, wherein the step of administering comprises administering the composition or pharmaceutical composition intravenously, intraparentally, topically, irrigation of wounds either as wound dressing or in sterile solution, intradermally, intramucosally, subcutaneously, sublingually, orally, intravaginally, intramuscularly, intracavernously, intraocularly, intranasally, into a sinus, intrarectally, intracranially, gastrointestinally, intraductally, intrathecally, subdurally, extradurally, intraventricular, intrapulmonary, into an abscess, intra articularly, into a bursa, subpericardially, into an axilla, intrauterine, into the pleural space, or intraperitoneally.

The disclosure also relates to a method of manufacturing a modified NK cell or modifying primary human lymphocyte population the method comprising:
(a) culturing one or a plurality of isolated lymphocytes;
(b) isolating the one or plurality of cells into a population of cells that exhibit from about 1.0% to about 99% CD 16 and from about 1.0% to about 99% CD52 from the one or plurality of lymphocytes as measured by flow cytometry;
(c) transducing the population of the one or plurality of isolated cells with one or a plurality of vectors comprising one or more isolated nucleic acid sequences disclosed herein.

In some embodiments, the method further comprises transducing the one or plurality of isolated cells with one or a plurality of vectors comprising one or more nucleic acid sequences encoding one or a combination of cytokines chosen from: IL-2, IL-12, IL- 15, IL-18, IL-21. In some embodiments, the method further comprises isolating a sample of lymphocytes from an umbilical cord tissue prior to step (a). In some embodiments, the method further comprises wherein steps (a) through (d) are performed ex vivo in a sterile chamber. In some embodiments, the method further comprises administering to a subject one or a plurality of modified T cells expressing one or a plurality of receptor molecules capable of binding one or a combination of tumor antigens chosen from amino acid sequences at least 70% homolgous to H3K27M, DNAJB1-PRKACA, bcr-abl, CDK4, MUM1, CTNNB1, CDC27, TRAPPC1, TPI, ASCC3, HHAT, FN1, OS-9, PTPRK, CDKN2A, HLA-A11, GAS7, SIR2, Prdx5, CLPP, PPPIR3B, EF2, ACTN4, ME1, NF-YC, HSP70-2, KIAA1440, CASP8, gag, pol, nef, env, survivin, MAGEA4, SSX2, PRAME, NYESO1, Oct4, Sox2, Nanog, WT1, p53, or MYCN.

In some embodiments, the disclosure relates to a method of manufacturing a modified NK cell or modifying a mononuclear cell with the method comprising:
(a) culturing one or a plurality of mononuclear cells;
(b) expanding the NK cells in culture;
(c) transducing the one or plurality of NK cells with one or a plurality of vectors comprising one or more isolated nucleic acid sequences disclosed herein.

In some embodiments, the method further comprises (d) transducing the one or plurality of NK cells with one or a plurality of vectors comprising one or more nucleic acid sequences encoding one or more cytokines chosen from: IL-2, IL-12, IL-15, IL-18, IL-21.

In some embodiments, the method further comprises isolating the one or plurality of mononuclear cells from one or a plurality of samples. In some embodiments, all of the steps are performed ex vivo in a sterile chamber. In some embodiments, the method further comprises isolating the one or plurality of NK cells after the transducing. In some embodiments, the step of isolating is accomplished by magnetic beads comprising a surface immobilized with a ligand for CD19. In some embodiments, the method further comprising freezing the cells at or lower than -80 degrees.

### BRIEF DESCRIPTION OF DRAWINGS

FIG. 1 is a schematic depicting the effects of TGFβ binding to the receptor complex in untransduced NK cells (UT), RBDNR-transduced NK cells (RBDNR), NKA-transduced NK cells (NKA), or NKCT-transduced NK cells (NKCT).

FIG. 2A vector maps of RBDNR (top), NKA (middle), and NKCT (bottom) constructs. FIG. 2B Flow cytometry demonstrating transduction efficiency based on TGFβRII and/or CD19 positive staining. Representative flow dot plots and histograms are on the right, and summarizing data on the left. FIG. 2C the phenotype of transduced and untransduced NK cells were examined by flow cytometry, and mean fluorescent intensity values for a given surface receptor is depicted in each panel. FIG. 2D Transduced and untransduced NK cells were stained with CFSE, and stimulated with irradiated feeder cells. After 3 days, cells were harvested and assessed for CFSE dilution by flow cytometry. FIG. 2E⁵¹Cr-labeled K561- target cells were co-cultured at various effector:target ratios with transduced or untransduced NK cells, and cytotoxicity after 5 hour co-culture was determined based on chromium content in the supernatant, calculated with spontaneous and maximum release controls All data is representative of >8 experiments, with * indicating significant p values <0.05.

FIG. 3A flow cytometry was performed to examine the expression of phosphorylated Smad2/3 in transduced and untransduced NK cells after 0.5, 1, and 3 hrs of exposure to 10 ng/mL TGFβ. Representative histograms are on top, and summarizing data below. FIG. 3B protein was isolated from transduced and untransduced NK cells after 1 hr of exposure to 10 ng/mL TGFβ, and was assessed for phosphorylated Smad2, phosphorylated Smad3, and Smad2 protein content by multiplex assay. Representative protein data for NK cells generated from one donor line. FIG. 3C Summarizing protein data for NK cells, where protein amounts are normalized to that of non-TGFβ conditions. All data is representative of >3 experiments, with * indicating significant p values <0.05.

FIG. 4A transduced and untransduced NK cells were exposed to TGFβ for 5 days, after which they were harvested and examined for phenotypic changes by flow cytometry. Representative histograms on the left and summarizing data on the right demonstrates changes in the expression of DNAM 1 and NKG2D, with mean fluorescent intensities normalized to that of non-TGFβ conditions. FIG. 4B⁵¹Cr-labeled SHSY5Y neuroblastoma cells were co-cultured at various effector:target ratios with transduced or untransduced NK cells, and cytotoxicity after 5 hour co-culture was determined based on chromium content in the supernatant, calculated with spontaneous and maximum release controls. All data is representative of >5 experiments, with * indicating significant p values <0.05.

FIG. 5A flow cytometry was performed to examine the expression of p65 (RELA) in transduced and untransduced NK cells after 0.5, 1, and 3 hrs of exposure to 10 ng/mL TGFβ. FIG. 5B Protein was isolated from transduced and untransduced NK cells after 1 hr of exposure to 10 ng/mL TGFβ, and was assessed for phosphorylated ERK1/2 and phosphorylated Akt protein content by multiplex assay. Summarizing protein data is graphed, where protein amounts are normalized to that of non-TGFβ conditions. All data is representative of >3 experiments, with * indicating significant p values <0.05.

FIG. 6A is a schematic for the in vivo neuroblastoma model: immunodeficient mice were preconditioned, inoculated with luciferase-positive SHSY5Y, treated with systemically delivered transduced or untransduced NK cells, and received adjuvant IL2. FIG. 6B shows tumor growth was monitored by evaluation bioluminescence of animals, which was FIG. 6C quantified by total photon counts taken at the same scale. FIG. 6D shows the effect of treatment with transduced or untransduced NK cells on animal survival over the length of the study. FIG. 6E peripheral blood was obtained 6 and 32 days following NK cell treatment, and was assessed to quantify the presence of genetic content from NK cells via ddPCR. Tumor bioluminescence was qualitatively identified according to the heat map color scale, in vivo results are representative with n=4 animals/experimental group, * indicates significant p values <0.05 compared to untreated animals, and NK cell identification was normalized to copies of TBP housekeeping gene content.

FIG. 7A is a schematic for the in vivo neuroblastoma model: immunodeficient mice were preconditioned, inoculated with luciferase-positive SHSY5Y, treated with systemically delivered transduced or untransduced NK cells on a weekly basis for 5 weeks, and received adjuvant IL2. FIG. 7B tumor growth was monitored by evaluation bioluminescence of animals, which was FIG. 7C quantified by total photon counts taken at the same scale. FIG. 7D shows the effect of treatment with transduced or untransduced NK cells on animal survival over the length of the study. Tumor bioluminescence was qualitatively identified according to the heat map color scale, in vivo results are representative with n=4 animals/experimental group, * indicates significant p values <0.05 compared to untreated, UT and Mock-tdx animals, and # indicates significant p values <0.05 compared to untreated animals only.

FIGS. 8A and Bshows SHSY5Y neuroblastoma line produced high levels of TGFβ in vivo from SHSY5Y-inoculated NSG mice.

FIG. 9 shows protection from the cytolytic activity of exogenous TGFβ as well as TGFβ-producing tumors in vitro was lost when TGFβ receptor-modified NK cells were placed in superphysiological (>50 ng/mL) environments.

FIG. 10 shows the RBDNR vector map and sequence.

FIG. 11 shows the NKA vector map and sequence.

FIG. 12 shows the NKCT vector map and sequence.

FIG. 13 depicts TGFB signaling in untransduced versus RBDNR, NKA, or NKCT TGFβ receptor-modified NK cells. Schematic depicting the effects of TGFβ binding to the receptor complex: Untransduced (UT) NK cells express the wild-type TGFBR.II, which, when engaged with TGFβ in the tumor microenvironment, initiates a signaling cascade that culminates in impaired NK-cell phenotype and cytotoxicity . NK cells transduced with the RBDNR, NKA, or NKCT variant TGFβ receptors alter the intracellular signaling and allow for maintained or enhanced NK cell phenotype and Q7 cytotoxicity in the setting of tumor-associated TGFβ.

FIGS. 14A –FIG. 14E. Generating and characterizing TGFβ receptor-modified NK cells. 14A, Vector maps of RBDNR (top), NKA (middle), and NKCT (bottom) constructs. 14B, Flow cytometry demonstrating transduction efficiency based on TGFβRII and/ or CD19-positive staining. Representative flow dot plots and histograms are on the right, and summarizing data on the left 14C, The phenotype of transduced and untransduced NK cells were examined by flow cytometry, and mean fluorescent intensity values for a given surface receptor is depicted in each panel. 14D, Transduced and untransduced NK cells were stained with CFSE, and stimulated with irradiated feeder cells. After 3 days, cells were harvested and assessed for CFSE dilution by flow cytometry. 14E, 51Cr-labeled K562 target cells were cocultured at various effector:target (E:T) ratios with transduced or untransduced NK cells, and cytotoxicity after 5-hour coculture was determined on the basis of chromium content in the supernatant, calculated with spontaneous and maximum release controls. All data is representative of experiments with > 8 donor lines, with * indicating significant P values < 0.05.

FIGS. 15A – 15C. Examining the molecular effects of TGFβ signaling 15A, Flow cytometry was performed to examine the expression of phosphorylated Smad2/3 in transduced and untransduced NK cells after 0.5, 1, and 3 hours of exposure to 10 ng/mL TGFβ. Representative histograms are on top, and summarizing data below. 15B, Protein was isolated from transduced and untransduced NK cells after 1 hour of exposure to 10 ng/mL TGFβ, and was assessed for phosphorylated Smad2, phosphorylated Smad3, and Smad2 protein content by multiplex assay. Representative protein data for NK cells generated from one donor line. 15C, Summarizing protein data for NK cells, where protein amounts are normalized to that of non-TGFβ conditions. All data is representative of experiments with >3 donor lines, with * indicating significant P values <0.05

FIGS. 16A – 16C. Examining downstream phenotypic and functional effects of TGF signaling. 16A, Transduced and untransduced NK cells were exposed to TGF for 5 days, after which they were harvested and examined for phenotypic changes by flow cytometry. Representative histograms on the left and summarizing data on the right demonstrates changes in the expression of DNAM1 and NKG2D, with mean fluorescent intensities normalized to that of non-TGF conditions . 16B, 51Cr-labeled SHSY5Y neuroblastoma cells were cocultured at various effector:target ratios with transduced or untransduced NK cells, and cytotoxicity after 5-hour coculture was determined on the basis of chromium content in the supernatant, calculated with spontaneous and maximum release controls. 16C, Cytotoxicity of NK cells against SHSY5Y neuroblastoma at a 40:1 effector:target ratio. All data is representative of experiments with < 7 donor lines, with * indicating significant P values <0.05.

FIGS. 17A – 17E. Long-term tumor-free survival with repeat doses of NK-cell treatment in vivo. 17A, Schematic for our in vivo neuroblastoma model, immunodeficient mice were preconditioned, inoculated with luciferase-positive SHSY5Y, treated with systemically delivered transduced or untransduced NK cells on a weekly basis for 5 weeks, and received adjuvant IL2. 17B, Tumor growth was monitored by evaluation bioluminescence of animals, which was quantified by total photon counts taken at the same scale (17C). 17D, The effect of treatment with transduced or untransduced NK cells on animal survival over the length of the study. 17E, Untransduced or transduced NK cells were identified using ddPCR methods to identify transgene copies in systemic blood isolated at weekly intervals following the last NK treatment. Tumor bioluminescence was qualitatively identified according to the heat map color scale, in vivo results are representative with n ¼ 5 – 9 animals/ experimental group; ^{Λ} indicates significant P values < 0.05 compared with RBDNR and NKCT animals, * indicates significant P values <0.05 compared with untreated, UT and Mock-tdx animals, and # indicates significant P values < 0.05 compared with untreated animals only.

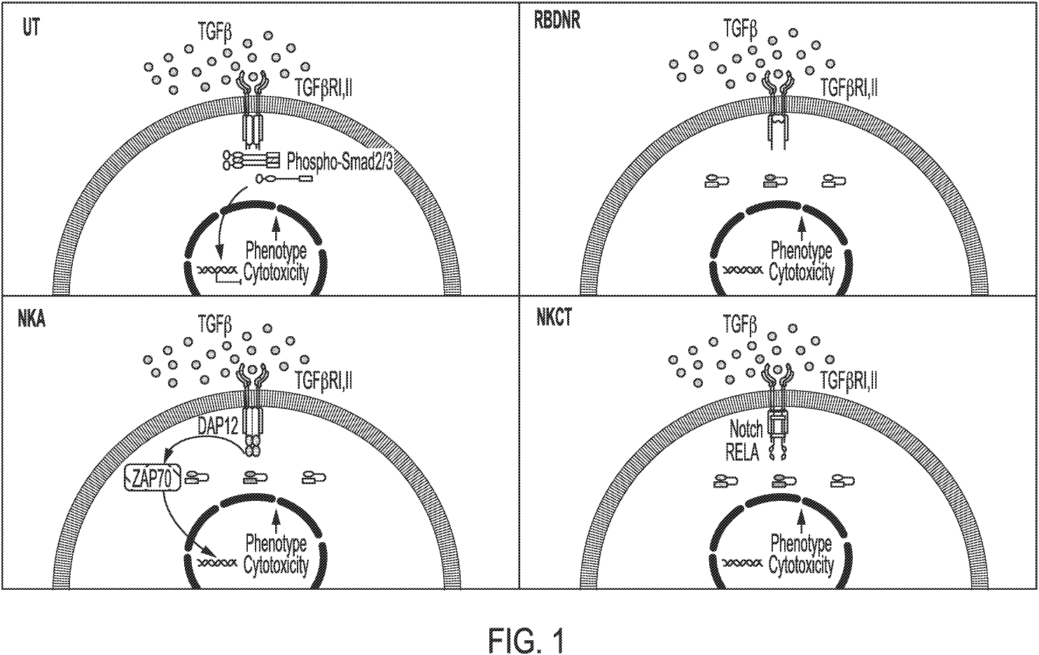

### DETAILED DESCRIPTION OF THE INVENTION

It has been discovered that genetically modifying allogeneic KIR-mismatched NK cells with one of three variants of a TGFβreceptor prevents downstream signaling leading to NK cell dysfunction (e.g., impaired proliferation, impaired cytolytic activity, exhaustion) and additionally incorporates activation signals, to turn this into immunological “switch”. Thus, the disclosed “inhibitory-to-activating switch” receptors represent a unique modification that takes advantage of a tumor-abundant cytokine and converts a customarily inhibitory environment into a therapeutically advantageous environment. This strategy provides, in part, gene-modified NK cells as a treatment modality for patients with neuroblastoma and other malignancies that utilize TGFβ secretion as a potent immune evasion mechanism.

### Definitions

Unless defined otherwise, all technical and scientific terms used herein have the same meaning as commonly understood by one of ordinary skill in the art to which the invention pertains.

The term “a” and “an” refers to one or to more than one (i.e, to at least one) of the grammatical object of the article. By way of example, “an element” means one element or more than one element.

The term “allogeneic” as used herein refers to medical therapy in which the donor and recipient are different individuals of the same species.

The term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

The term “antigen presenting cell (ARC)” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

The term “autologous” as used herein refers to medical therapy in which the donor and recipient are the same person.

The term “cord blood” as used herein has its normal meaning in the art and refers to blood that remains in the placenta and umbilical cord after birth and contains hematopoietic stem cells . Cord blood may be fresh, cryopreserved, or obtained from a cord blood bank.

The term “cytokine” as used herein has its normal meaning in the art. Nonlimiting examples of cytokines used in the invention include IL-2, 11.-6, IL-7, IL-12, IL-15, IL-18, IL-21, and IL-27.

The term “cytotoxicity” as used herein is meant to refer to the extent of the destructive or killing capacity of an agent. In certain embodiments, NK cell cytotoxicity is meant to refer to the character of the NK cell activity that limits the development of cancer cells. Cytotoxic potential can be expressed as the percent of target cell death above background (e.g., without the binding molecule or with an irrelevant binding molecule), using complete target cell death as 100% In certain aspects, the NK cell engineered accordingly to the disclosure reduces the quantity, number, amount or percentage of targeted cancerous cells by at least 25%, at least 30%, at least 40%, at least 50%, at least 65%, at least 75%, at least 85%, at least 95%, or at least 99% (to undetectable level) in a subject relative to a negative control. Cytotoxicity of the engineered NK cells of the invention may be monitored by in vitro assay: by using cytotoxicity such as used currently when T cells are tested; a classical protocol is described in the section “General methods” thereafter; by in vivo assay: by using for instance the tumor challenge test in mammals such as mice, for instance disclosed in Ng S, Yoshida K, Zelikoff JT 2010 “tumor challenges in immunotoxicity testing”, Methods Mol Biol.;598:143-55 Reduction of immune checkpoint activity such as PD-1 may be monitored: in vitro assay: cytotoxicity or serial killing assay may be used (Bhat R and Watzl C 2007 “Serial Killing of Tumor Cells by Human Natural Killer Cells - Enhancement by Therapeutic Antibodies”, PLoS ONE.; 2(3); by in vivo assay: survival curve with tumor-expressing mammals such as mice may be used (Valiathan C and McFaline J L, 2011, “A Rapid Survival Assay to Measure Drug- Induced Cytotoxicity and Cell Cycle Effects” DNA Repair (Amst). PMC 2013 Jan 2).

The term “cytolytic activity” is meant to refer to the ability of NK cells to initiate an immediate and direct cytolytic response to, e.g., virally infected or malignantly transformed cells.

The term “cytotoxic T-cell” or “cytotoxic T lymphocyte” as used herein is a type of immune cell that bears a CD8+ antigen and that can kill certain cells, including foreign cells, tumor cells, and cells infected with a virus. Cytotoxic T cells can be separated from other blood cells, grown ex vivo, and then given to a patient to kill tumor or viral cells. A cytotoxic T cell is a type of white blood cell and a type of lymphocyte.

The term “dendritic cell” or “DC.” as used herein describes a diverse population of morphologically similar cell types found in a variety of lymphoid and non-lymphoid tissues, see Steinman, Ann. Rev. Immunol. 9:271-296 (1991).

The term “effector cell” as used herein describes a cell that can bind to or otherwise recognize an antigen and mediate an immune response. Tumor, virus, or other antigen-specific T-cells and NKT-cells are examples of effector cells.

The term “endogenous” as used herein refers to any material from or produced inside an organism, cell, tissue or system.

The term “engraflment” (or transplantation) as used herein it is meant to refer to a process by which transplanted or transfused cells, e.g. NK cells, from an allogeneic donor grow and reproduce with a recipient.

The term “epitope” or “antigenic determinant” as used herein refers to the part of an antigen that is recognized by the immune system, specifically by antibodies, B cells, or T cells

The term “exogenous” as used herein refers to any material introduced from or produced outside an organism, cell, tissue or system.

The term “HLA” as used herein refers to human leukocyte antigen. There are 7,196 HLA alleles. These are divided into 6 HLA class 1 and 6 HLA class II alleles for each individual (on two chromosomes) . The HLA system or complex is a gene complex encoding the major histocompatibility complex (MHC) proteins in humans. HLAs corresponding to MHC Class I (A, B, or C) present peptides from within the cell and activate CD8-positive (ie., cytotoxic) T-cells. HLAs corresponding to MHC Class II (DP, DM, DOA, DOB, DQ and DR) stimulate the multiplication of CD4-positive T-cells) which stimulate antibody-producing B-cells.

The term “isolated” as used herein means separated from components in which a material is ordinarily associated with, for example, an isolated cord blood mononuclear cell can be separated from red blood cells, plasma, and other components of cord blood.

As used herein, “Natural Killer cell” (“NK cell”) refers to a type of cytotoxic lymphocyte of the immune system NK cells provide rapid responses to virally infected cells and respond to transformed cells.

The term “activity of NK cells” refers, in part, to NK cell activity in promoting antitumor immunotherapy (cytotoxic or cytolytic activity); as regulatory cells engaged in reciprocal interactions with other immune cells (such as immune checkpoints); in improving hematopoietic and solid organ transplantation (engraftment). In certain embodiments, the “activity of NK cells” refers to the therapeutic activity of NK cells.

A “peptide library” or “overlapping peptide library” as used herein within the meaning of the application is a complex mixture of peptides which in the aggregate covers the partial or complete sequence of a protein antigen, especially those of opportunistic viruses. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries are commercially available and may be custom-made for particular antigens. Methods for contacting, pulsing or loading antigen-presenting cells are well known and incorporated by reference to Ngo, et al (2014), Peptide libraries may be obtained from JPT and are incorporated by reference to the website at https://www.jpt.com/products/peptrack/peptide-libraries.

A “peripheral blood mononuclear cell” or “PBMC” as used herein is any peripheral blood cell having a round nucleus. These cells consist of lymphocytes (T cells, B cells, NK cells) and monocytes. In humans, lymphocytes make up the majority of the PBMC population, followed by monocytes, and only a small percentage of dendritic cells.

The term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines

A “subject” or “host” or “patient” as used herein is a vertebrate, preferably a mammal, more preferably a human. Mammals include, but are not limited to humans, simians, equines, bovines, porcines, canines, felines, murines, other farm animals, sport animals, or pets. Humans include those in need of virus- or other antigen-specific T-cells, such as those with lymphocytopenia, those who have undergone immune system ablation, those undergoing transplantation and/or immunosuppressive regimens, those having naive or developing immune systems, such as neonates, or those undergoing cord blood or stem cell transplantation. In a typical embodiment, the term “patient” as used herein refers to a human.

A “T-cell population” or “T-cell subpopulation” is intended to include thymocytes, immature T lymphocytes, mature T lymphocytes, resting T lymphocytes and activated T-lymphocytes. The T-cell population or subpopulation can include αβ T-cells, including CD4+ T-cells, CD8+ T cells, γδ T-cells, Natural Killer T-cells, or any other subset of T-cells.

The terms “treatment” or “treating” as used herein is an approach for obtaining beneficial or desired results including clinical results For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival.

The terms “vector” or “vectors” as used herein are meant to refer to a nucleic acid molecule capable of transporting another nucleic acid to which it has been linked. A “vector” includes, but is not limited to, a viral vector, a plasmid, a RNA vector or a linear or circular DNA or RNA molecule which may consists of a chromosomal, non chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adeno-associated viruses), coronavirus, negative strand NA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lenti- virus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non-limiting example, that are able to integrate the genome of a target cell. At the opposite by “non integrative lentiviral vectors (or NILV)” is meant efficient gene delivery vectors that do not integrate the genome of a target cell through the action of the virus integrase.

Delivery vectors and vectors can be associated or combined with any cellular permeabilization techniques such as sonoporation or electroporation or derivatives of these techniques. By “cell” or “cells” is intended any eukaryotic living cells, primary cells and cell lines derived from these organisms for in vitro cultures.

### Hematopoietic Cells

Hematopoietic cells useful in the methods disclosed herein can be any hematopoietic cells able to differentiate into NK cells, e.g., precursor cells, hematopoietic progenitor cells, hematopoietic stem cells, or the like. Hematopoietic cells can be obtained from tissue sources such as, e.g., bone marrow, cord blood, placental blood, peripheral blood, liver or the like, or combinations thereof. In some embodiments, engineered NK cells described herein may be produced from hematopoietic cells, e.g., hematopoietic stem or progenitors from any source, e.g., placental tissue, placental perfusate, umbilical cord blood, placental blood, peripheral blood, spleen, liver, or the like. In some embodiments, the hematopoietic cells, e.g., hematopoietic stem cells or progenitor cells, from which the engineered NK cells described herein are produced, are obtained from placental perfusate, umbilical cord blood or peripheral blood. In one embodiment, the hematopoietic cells, e.g., hematopoietic stem cells or progenitor cells, from which the engineered NK cells described herein are produced, are combined cells from, e.g. placental perfusate and cord blood, e.g., cord blood from the same placenta as the perfusate.

In some embodiments, the hematopoietic cells are CD34+ cells. In specific embodiments, the hematopoietic cells are CD34+CD38+ or CD34+CD38..... In another embodiment, the hematopoietic cells are CD34+CD38-Lin-. In another embodiment, the hematopoietic cells are one or more of CD2-, CD3-,CD11b...., CD 11c..., CDI4..., CD16---, CD19--, CD24---, CD56---, CD66b... and/or glycophorin A-. In another embodiment, the hematopoietic cells are CD2 -, CD3-, CD11b-, CD11c-, CD14-, CD16-, CD19-. CD24-, CD56-, CD66b- and glycophorin A-. In another embodiment, the hematopoietic cells are CD34+CD38-CD33-CDlI7-. . In another more specific embodiment, the hematopoietic cells are CD34+CD38-CD33-CD117-CD235-CD36

In another embodiment, the hematopoietic cells are CD45+. In another specific embodiment, the hematopoietic cells are CD34+CD454+.In another embodiment, the hematopoietic cell is Thy-1 +. In a specific embodiment, the hematopoietic cell is CD34-+Thy-1 +. In another embodiment, the hematopoietic cells are CD133+. In specific embodiments, the hematopoietic cells are CD34+CD133+ or CD133+Thy-1+.

In certain other embodiments, the CD34+cells are CD45-

In certain embodiments, the hematopoietic cells are CD34-

In some embodiments, the hematopoietic cells can also lack certain markers that indicate lineage commitment, or a lack of developmental naiveté. For example, in another embodiment, the hematopoietic cells are HLA-DR-. In specific embodiments, the hematopoietic cells are CD34+HLA-DR-, CD133+HLA-DR-, Thy-1+HLA-DR- or ALDH+HLA+DR- In another embodiment, the hematopoietic cells are negative for one or more, preferably all, of lineage markers CD2, CD3, CD11b,CD11c, CD14, CD16, CD19, CD24, CD56, CD66b and glycophorin A.

Thus, hematopoietic cells can be selected for use in the methods disclosed herein on the basis of the presence of markers that indicate an undifferentiated state, or on the basis of the absence of lineage markers indicating that at least some lineage differentiation has taken place. Methods of isolating cells, including hematopoietic cells, on the basis of the presence or absence of specific markers is discussed below.

Hematopoietic cells used in the methods provided herein can be a substantially homogeneous population, eg., a population comprising at least about 95%, at least about 98% or at least about 99% hematopoietic cells from a single tissue source, or a population comprising hematopoietic cells exhibiting the same hematopoietic cell-associated cellular markers. For example, in various embodiments, the hematopoietic cells can comprise at least about 95%, 98% or 99% hematopoietic cells from bone marrow, cord blood, placental blood, peripheral blood, or placenta, e.g., placenta perfusate.

Hematopoietic cells used in the methods provided herein can be obtained from a single individual, e.g., from a single placenta, or from a plurality of individuals, e.g., can be pooled. Where the hematopoietic cells are obtained from a plurality of individuals and pooled, the hematopoietic cells may be obtained from the same tissue source. Thus, in various embodiments, the pooled hematopoietic cells are all from placenta, e.g., placental perfusate, all from placental blood, all from umbilical cord blood, all from peripheral blood, and the like

Hematopoietic cells used in the methods disclosed herein can, in certain embodiments, comprise hematopoietic cells from two or more tissue sources. The hematopoietic cells from the sources can be combined in any ratio, for example. 1:10, 2:9, 3:8, 4:7, 5:6, 6:5, 7:4, 8:3, 9:2, 1:10, 1:9, 1:8, 1:7, 1:6, 1:5, 1:4, 1:3, 1:2, 1:1, 2:1, 3-1, 4:1, 5:1, 6:1, 7: 1, 8:1 or 9:1.

### Engineered Natural Killer (NK) Cells Expressing Novel TGF-Beta (TGFb) Receptors

Provided are immune cells, and in particular Natural Killer (NK) cells, that are engineered to express or contain molecules, such as recombinant or engineered molecules or a functional and/or catalytically-active portion or variant thereof, which are involved in or capable of modulating, e.g., promoting, inducing, enhancing, inhibiting, preventing, or carrying out or facilitating, NK-cell activity. In some embodiments, the composition comprises a cell expressing a chimeric protein comprising a first domain and a second domain, and optionally a third domain, wherein the first domain is a TGFβ receptor extracellular domain and the second domain is a NK-activating domain and the third domain is a selection domain, the slection domain expressing one or a combination of CD molecules. In some embodiments, the third domain is a modified CD19 moelcule. In some embodiments, the second domain is either a DAP12 seqeunce or a functional fragment thereof or a transmembrane domain chosen from the below Table Z:

TABLE Z

| | | | | | | | | | | | | | | | | | | | | |
|---|---|---|---|---|---|---|---|---|---|---|---|---|---|---|---|---|---|---|---|---|
| TMD aa | 1 | 2 | 3 | 4 | 5 | 6 | 7 | 8 | 9 | 10 | 11 | 12 | 23 | 14 | 15 | 16 | 17 | 18 | 19 | 20 |
| KIR2D S1 | V | L | 1 | G | T | S | V | V | K | I | P | F | T | I | L | L | F | F | L | |
| KIR2D S2 | v | L | I | G | T | S | V | V | K | I | P | F | T | I | L | L | F | F | L | L |
| KlR2D S3 | V | L | I | G | T | S | V | V | K | L | P | F | T | I | L | L | F | F | L | |
| KIR2D S4 | V | L | I | G | T | S | V | V | K | I | P | F | T | I | L | L | F | F | L | L |
| KIR2D S5 | V | L | I | G | T | S | V | V | K | L | P | F | T | I | L | L | F | F | L | |
| KIR3D S1 | I | L | I | G | T | S | V | V | K | I | P | F | T | I | L | L | F | F | L | L |
| NK-p44 | L | V | P | v | F | C | G | L | L | V | A | K | S | L | S | A | L | L | V | |
| NKG2 C | L | T | A | E | V | L | G | I | I | C | I | V | L | M | A | T | V | L | K | T |
| NKG2E | L | T | A | E | V | L | G | I | I | C | I | V | L | M | A | T | V | L | K | T |

Where the column number at the top represents amino acid number 1 through 20 in sequence from amino to carboxy orientation. Each seqeunce in a row from top to bottom is SEQ ID NO:17, SEQ ID NO: 18, SEQ ID NO: 19, SEQ ID NO:20, SEQ ID NO:21, SEQ ID NO:22, SEQ ID NO:23, SEQ ID NO:24, and SEQ ID NO:25. Note that the compositions of the disclosure comprise nucleic acid sequences such as DNA, RNA or DNA/RNA hybrid molecules that express one of the transmembrane sequences from the Table Z One of ordinary skill in the art would know how to identify a DNA seqeunce encoding the above amino acid sequences by running a EXPASY function to identify the series of codons for each amino acid capable of encoding the sequential sequence.

The disclosure relates to a cell expressing any one or plurality of nucleic acids disclosed herein. The disclosure relates to a cell expressing any one or plurality of amino acid sequences disclosed herein or encoded by any of the one or plurality of nucleic acids disclosed herein. In some embodiments, the cell is a NK cell. In some embodiments, the NK cell is from a healthy subject or a subject not diagnosed with a hyperproliferative disorder such as a cancer. In some embodiments, the NK cell is a

In some embodiments, the disclosure relates to a composition or pharmaceutical composition comprising a modified and/or isolated NK cell comprising any one or plurality of nucleic acids disclosed herein, and/or expressing any one or plurality of amino acid sequences disclosed herein or encoded by any of the one or plurality of nucleic acids disclosed herein. In some embodiments, the amino acid sequence is a chimeric protein comprising, consisting of or consisting essentially of a TGFbeta receptor domain, capable of binding TGFbeta when exposed to TGFbeta in vivo or in culture more than unmodified cells expressing a wild-type TGFbeta receptor; and a NK-cell activation domain, capable or inducing activation of the NK cell on which the domain is expressed In some embodiments, the NK-activation domain comprises DAP12 or a functional fragment thereof. In some embodiments, the the NK-activation domain comprises DAP 12 or a functional fragment thereof In some embodiments, the NK-activation domain comprises a sequence that is capable of activating endogenously expressed DAP12 in the cell upon which the chimeric protein is expressed. In some embodiments, the NK-activation domain comprises SEQ ID NO: 17, 18, 19, 20, 21, 22, 23, 24 or 25 or a functional fragment thereof that comprises at least about 70%, 75%, 80%, 85%, 90%, 91%, 92%, 93%, 94%, 95%, 96%, 97%, 98%, 99% sequence identity to SEQ ID NO: 17, 18, 19, 20, 21, 22, 23, 24 or 25. In some embodiments, the a functional fragment thereof that comprises at least about 70%, 75%, 80%, 85%, 90%, 91%, 92%, 93%, 94%, 95%, 96%, 97%, 98%, 99% sequence identity to SEQ ID NO: 17, 18, 19, 20, 21, 22, 23, 24 or 25 that has the lysine at position 9, 10 or 19 corresponding to the order of amino acids in Table Z, as oriented in the carboxy to amino orientation. In some embodiments, the second domain comprises a functional fragment thereof that comprises about 70%, 75%, 80%, 85%, 90%, 91%, 92%, 93%, 94%, 95%, 96%, 97%, 98%, 99% sequence identity to one or a combination of SEQ ID NO: 17, 18, 19, 20, 21, 22, 23, 24 or 25. In some embodiments, the second domain comprises a functional fragment thereof that comprises about 70%, 75%, 80%, 85%, 90%, 91%, 92%, 93%, 94%, 95%, 96%, 97%, 98%, 99% or 100% sequence identity to one or a combination of SEQ ID NO: 17, 18, 19, 20, 21, 22, 23, 24 or 25.

In some embodiments the second domain comprises an amino acid encoded by SEQ ID NO: 9 optionally also comprising an amino acid encoded by SEQ ID NO:7 and/or SEQ ID NO:8, or a functional fragment thereof that comprises about 70%, 75%, 80%, 85%, 90%, 91%, 92%, 93%, 94°0. 95%, 96%, 97%, 98%, 99% sequence identity to one or a combination of SEQ ID NO: 7 or 8.

In some embodiments, the first domain comprises an amino acid encoded by SEQ ID NO: 4 optionally also comprising an amino acid encoded by SEQ ID NO:5 and/or SEQ ID NO:6, or a functional fragment thereof that comprises about 70%, 75%, 80%, 85%, 90%, 91%, 92%, 93%, 94%, 95%, 96%, 97%, 98%, 99% sequence identity to one or a combination of SEQ ID NO: 5 or 6. In some embodiments, the second domain comprises a nucleic acid that that comprises about 70%, 75%, 80%, 85%, 90%, 91%, 92%, 93%, 94%, 95%, 96%, 97%, 98%, 99% or 100% sequence identity to one or a combination of SEQ ID NO: 4, 5 and/or 6. In some embodiments, the first domain comprises a nucleic acid that that comprises about 70%, 75%, 80%, 85%, 90%, 91%, 92%, 93%, 94%, 95%, 96%, 97%, 98%, 99% or 100% sequence identity to SEQ ID NO:4 but is free of a functional SEQ ID NO:6.

In some embodiments, the cell expresses a nucleic acid expressed herein or a chimeric protein disclosed herein for no longer than about 5, 10, 15, 20, 30, or 40 days after administration.

In some embodiments, the cell expresses a nucleic acid expressed herein or a chimeric protein disclosed herein, but the expression is not constituitive for more than about 5, 10, 15, 20, 30, or 40 days after administration.

In some embodiments, the chimeric protein or fusion protein comprises a linker between the first and second and/or between the second and third amino acid. In some embodiments, the linker is encoded by the T2A nucleic acid seqeunce disclosed herein.

In some embodiments, the nucleic acid sequences disclosed herein comprise a nucleic acid sequence that encodes an interleukin molecule or a functional fragment thereof. In some embodiments, the nucleic acid sequences disclosed herein comprise a nucleic acid seqeunce that encodes an interleukin chosen from one or a combination of those in Table YY.

Cytokine Table YY

| SEQ ID | DESCRIPTION | UniProtKB4# |
|---|---|---|
| 26 | Human Interleukin 2 | P60568 |
| 27 | Human Interleukin 12A | P29459 |
| 28 | Human Interleukin 12B | P29460 |
| 29 | Human Interleukin 15 | p40933 |
| 30 | Human Interleukin 18 | Q14116 |
| 31 | Human Interleukin 21 | Q9HHE4 |

SEQ#26
>sp|P60568|IL2_HUMAN Interleukin-2 OS=Homo sapiens
OX=9606 GN=IL2 PE=I SV=1
MYRMQLLSCIALSLALVTNSAPTSSSTKKTQLQLEHLLLDLQMILNGINN
YKNPKLTRLTFKFYMPKKATELKHIQCLEEELKPLEEVLNLAQSKNFHLR
PRDLISNINVIVLELKGSETTFMCEYADETATIVEFLNRWITFCQSIIST
LT

SEQ#27
>SP|P29459|IL12A_HUMAN Interleukin-12 subunit
alpha OS=Homo sapiens OX=9696 GN=IL12A
PE=1 SV=2
MCPARSLLLVATLVLLDHLSLARNLPVATPDPGMFPCLHHSQNLLRAVSN
MLQKARQTLEFVPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS

SEQ#28
>sp|P29469|IL12B_HUMAN Interleukin-12 subunit
beta OS-Homo sapiens OX=9606 GN=IL12B
PE=1 SV=1
MCHQQLVISWFSLVFLASPLVAIWELKKDVYVVELDWVPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEVEVSVECQEDSACP
AAEESLPIEVMVDAVHKLKVENVTSSFFIRDIIKPDPPKNlQLKPLKNSR
QVEVSWEVPOTWSTPHSYFSLTFCVQVQGKSKREKKORVFTDKTSATVIC
RKNASISVRAQDRYYSSSWSEWASVPCS

SEQ#29
>sp|P40933|IL1S_HUMAN Interleukin-15
OS=Homo sapiens OX=9606 GN=ILI5 PE=1 SV=1
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

SEQ#30
>sp|Q14116|IL18_HUMAN Interleukin-18
OS=Homo sapiens OX=9606 GN=IL18 PE=1 SV=1
MAAEPVEDNCINFVAMKFIDNTLYFIAEODENLESDYFGKLESKLSVIRN
LNDQVLFIDQGNRPLFEDMTDSDCRDNAPRTIFIISMYKDKSQPRGMAVT
ISVKCEKISTLSCENKIISFKEMNPPDNIKDTKSDIIFFQRSVPGHDNKM
QFESSSYEGYFLACEKERDLFKLILKKEDELGDRSIMFTVQNED

SEQ#31
>sp|Q9HBE4|IL21_HUMAN Interleukin-21
OS=Homo sapiens OX=9606 GNIL21 PE=1 SV=3
MRSSPGNMERIVICLMVIFLGTLVHKSSSQGQDRHMIRMRQLIDIVDQLK
NVVNDLVPEFLPAPEDVETNCEWSAFSCFQKAQLKSANTGNNERIINVSI
KKLKRKPPSTNAGRRQKNRLTCPSCDSYEKKPPKEFLERFKSLLQKMIHQ
HLSSRTHGSEDS

In some embodiments, the cell expresses an amino acid domain comprises that comprises about 70%, 75%, 80%, 85%, 90%, 91%, 92%, 93%, 94%, 95%, 96%, 97%, 98%, 99% or 100% sequence identity to one or a combination of SEQ ID NO: 26 - 31 or a functional fragment that that comprises about 70%, 75%, 80%, 85%, 90%, 91%, 92%, 93%, 94%, 95%, 96%, 97%, 98%, 99% or 100% sequence identity to one or a combination of SEQ ID NO: 26 - 31.

In some embodiments, the methods disclosed herein comprise a step of administering a protein of that comprises about 70%, 75%, 80%, 85%, 90%, 91%, 92%, 93%, 94%, 95%, 96%, 97%, 98%, 99% or 100% sequence identity to one or a combination of SEQ ID NO: 26 - 31 or a nucleic acid encoding the same.

### Receptor 1

In some embodiments, the composition comprises a chimeric protein with a first domain and a second domain, and optionally a third domain, wherein the first domain is a TGFβ receptor extracellular domain and the second domain is a NK-activating domain. In some embodiments, the first domain is a TGFβ receptor coupled to the second domain which is the truncated TGFβ dominant negative receptor to tags for CD19, which allows users to specifically target cells containing this modified receptor for downstream application (such as silencing if needed), and we define it as the RBDNR receptor. The molecular structure of RBDNR is as follows: human type II. TGFβ receptor cDNA was truncated at nt597 and coupled to a truncated CD19 tag and pac puromycin resistance gene via T2A sequences.

### Receptor 2

This novel TGFβ receptor contains the truncated TGFβ dominant negative receptor coupled to tags for CD 19 and fused to the intracellular DNAX-activation protein 12 (DAP12) activation motif, and we define it as the NKA receptor. The DAP12 motif initiates cellular signaling through its single immuno-receptor tyrosine-based activation motif (ITAM) which in turn initiates a molecular signaling cascade that leads to cell activation. The molecular structure of NKA is as follows: human type II TGFβ receptor cDNA was truncated at nt597 and coupled to the transmembrane and intracellular coding region of DAP12 as derived from full-length DAP 12 cDNA, a truncated CD19 tag and a pac puromycin resistance gene via T2A sequences.

### Receptor 3

This novel TGFβ receptor contains the truncated TGFβ dominant negative receptor coupled to tags for CD19 and fused to a synthetic Notch-like receptor (“synNotch”) coupled to the RELA/p65 protein, and we define it as the NKCT receptor. Upon binding to TGFβ, the Notch-like receptor is cleaved, and RELA/p65 is translocated to the nucleus, where NFKB signaling directly leads to cell activation. The molecular structure of NKCT is as follows: human type II TGFβ receptor cDNA was truncated at nt597 and coupled to a “SynNotch” receptor composed of the Notch 1 minimal regulatory region fused to the DNA binding domain for RELA (p65), a VP64 effector domain, a truncated CD19 tag and a pac puromycin resistance gene via T2A sequences.

### Receptor 4

This novel TGFβ receptor contains the truncated TGFβ dominant negative receptor coupled to tag for CD19 and fused to the DNAX-activation protein 12 (DAP12) activation motif, and we define it as the NKA2 receptor. The DAP 12 motif initiates cellular signaling through its single immuno-receptor tyrosine-based activation motif (ITAM) which in turn initiates a molecular signaling cascade that leads to cell activation. The molecular structure of NKA2 is as follows: human type II TGFβ receptor cDNA was truncated at nt498 coupled to the coding region of DAP12 as derived from full-length DAP12 cDNA and a truncated CD19 tag via T2A sequence

### Receptor 5

This novel TCFβ receptor contains the truncated TGFβ dominant negative receptor coupled to tag for CD19 and fused to a truncated KIR2DS2 receptor, and we define it as the NKA3 receptor. The KIR2DS2 receptor initiates cellular signaling by recruiting DAP12 which through its single immuno-receptor tyrosine-based activation motif (ITAM) in turn initiates a molecular signaling cascade that leads to cell activation. The molecular structure of NKA3 is as follows: human type II TGFβ receptor cDNA was truncated at nt498 coupled to the coding region of KIR2DS2 as derived from KIR2DS2 cDNA from nt690 to nt912 and a truncated CD19 tag via T2A sequence.

With respect to suitable NK cells it is generally contemplated that the NK cells may be an autologous NK cell from a subject that will receive genetically modified NK cells Such autologous NK cells may be isolated from whole blood, or cultivated from precursor or stem cells using methods well known in the art Moreover, it should also be appreciated that the NK cells need not be autologous, but may be allogenic, or heterologous NK cells. However, in particularly preferred aspects of the inventive subject matter, the NK cells are genetically engineered to achieve one or more desirable traits. In some embodiments, suitable NK cells will also be continuously growing (‘immortalized’) cells.

### Production of Engineered Natural Killer Cells

### Isolation of Engineered NK Cells

Methods of isolating natural killer cells are known in the art and can be used to isolate the engineered NK cells Natural killer cells can be isolated or enriched by staining cells from a tissue source, e.g., peripheral blood, with antibodies to CD56 and CD3, and selecting for CD56+CD3- cells. The engineered NK cells can be isolated using a commercially available kit, for example, the NK Cell Isolation Kit (Miltenyi Biotec). The engineered NK cells can also be isolated or enriched by removal of cells other than NK cells in a population of cells that comprise the TSNK cells. For example, engineered NK cells cells may be isolated or enriched by depletion of cells displaying non-NK cell markers using, e.g., antibodies to one or more of CD3, CD4, CD14, CD19, CD20, CD36, CD66b, CD123, HLA DR and/or CD235a (glycophorin A). Negative isolation can be carried out using a commercially available kit, e.g., the NK Cell Negative Isolation Kit (Dynal Biotech), Cells isolated by these methods may be additionally sorted, e.g., to separate CD16+ and CD16-cells.

Cell separation can be accomplished by, e.g., flow cytometry, fluorescence-activated cell sorting (FACS), or, preferably, magnetic cell sorting using microbeads conjugated with specific antibodies. The cells may be isolated, e.g., using a magnetic activated cell sorting (MACS) technique, a method for separating particles based on their ability to bind magnetic beads (e.g., about 0.5-100 µm diameter) that comprise one or more specific antibodies, e.g., anti-CD56 antibodies. Magnetic cell separation can be performed and automated using, e.g., an AUTOMACS™ Separator (Miltenyi) A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

### Selection

In certain embodiments, greater than 50%, 60%, 70%, 80%, 90%, 92%, 94%, 96%, 98% of the engineered NK cells are CD16+. In other embodiments, at least 50%, 60%, 70%, 80%, 82%, 84%, 86%, 88% or 90% of said engineered NK cells are CD56^{dim.} In other embodiments, at least 50%, 52%, 54%, 56%, 58% or 60% of the engineered NK cells are cells are CD16+CD56^{dim.} In other embodiments, at least 50%, 52%, 54%, 56%, 58% or 60% of the engineered NK cells are cells are CD16+CD56⁺. In such embodiments, CD56 is often use for NK-cell positive selection. Miltenyi has a clinical grade kit for CD56 selection.

In certain embodiments, the engineered NK cells can be assessed by detecting one or more functionally relevant markers, for example, CD94, CD161, NKp44, DNAM-1, 2B4, NKp46, CD94, KIR, and the NKG2 family of activating receptors (e.g., NKG2D). In some embodiments, the purity of the isolated or enriched natural killer cells can be confirmed by detecting one or more of CD56, CD3 and CD16.

Optionally, the cytotoxic activity of the engineered natural killer cells can be assessed, e.g., in a cytotoxicity assay using tumor cells, e.g., cultured K562, LN-18, U937, WERI-RB-I, U-1 18MG, HT-29, HCC2218, KG-1, or U266 tumor cells, or the like as target cells.

### Transduction

### Cryopreserving Engineered NK Cells

Cells provided herein can be cryopreserved, e.g., in cryopreservation medium in small containers, e.g., ampoules or septum vials . In another embodiment, the method further comprises cryopreserving a population of NK cells. In one embodiment, the method comprises [INSERT METHOD HERE], further comprising the steps of cryopreserving the NK cells from step (***) in a cryopreservation medium. In a specific embodiment, they cryopreserving step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below –80° C.

In certain embodiments, cells provided herein are cryopreserved at a concentration of about 1 × 10⁴⁻⁵× 10⁸ cells per mL. In specific embodiments, cells provided herein are cryopreserved at a concentration of about 1 × 10⁶-1.5×10⁷ cells per mL. In more specific embodiments, cells provided herein are cryopreserved at a concentration of about 1 × 10⁴, 5×10⁴, 1 × 10⁵, 5 × 10⁵, 1 x 10⁶, 5×10⁶, 1× 10⁷, 1.5×10⁷ cells per mL.

Suitable cryopreservation medium includes, but is not limited to, normal saline, culture medium including, e.g., growth medium, or cell freezing medium, for example commercially available cell freezing medium, e.g., C2695, C2639 or C6039 (Sigma); CryoStor® CS2, CryoStor® CS5 or CryoStor®CS10 (BioLife Solutions). Cryopreservation medium preferably comprises DMSO (dimethylsulfoxide), at a concentration of, e.g., about 1, 2, 3, 4, 5, 6, 7, 8, 9 or 10% (v/v). Cryopreservation medium may comprise additional agents, for example, methylcellulose, dextran, albumin (e.g., human serum albumin), trehalose, and/or glycerol. In certain embodiments, the cryopreservation medium comprises about 1%-10% DMSO, about 25%-75% dextran and/or about 20-60% human serum albumin (HSA). In certain embodiments, the cryopreservation medium comprises about 1%-10% DMSO, about 25%-75% trehalose and/or about 20-60% human HSA. In a specific embodiment, the cryopreservation medium comprises 5% DMSO, 55% dextran and 40% HSA. In a more specific embodiment, the cryopreservation medium comprises 5% DMSO, 55% dextran (10% w/v in normal saline) and 40% HSA. In another specific embodiment, the cryopreservation medium comprises 5% DMSO, 55% trehalose and 40% HSA. In a more specific embodiment, the cryopreservation medium comprises 5% DMSO, 55% trehalose (10% w/v in normal saline) and 40% HSA. In another specific embodiment, the cryopreservation medium comprises CryoStor® CS5. In another specific embodiment, the cryopreservation medium comprises CryoStor®CS10.

Cells provided herein can be cryopreserved by any of a variety of methods, and at any stage of cell culturing, expansion or differentiation.

Cells provided herein are preferably cooled in a controlled-rate freezer, e.g., at about 0. i, 0.3, 0.5, or 1° C./min during cryopreservation. A preferred cryopreservation temperature is about –80° C. to about –180° C., preferably about –125° C. to about –140° C. Cryopreserved cells can be transferred to liquid nitrogen prior to thawing for use In some embodiments, for example, once the ampoules have reached about -90° C., they are transferred to a liquid nitrogen storage area. Cryopreserved cells preferably are thawed at a temperature of about 25° C. to about 40° C., preferably to a temperature of about 37° C. In certain embodiments, the cryopreserved cells are thawed after being cryopreserved for about 1, 2, 4, 6, 10, 12, 18, 20 or 24 hours, or for about 1. 2, 3, 4, 5, 6, 7, 8, 9, 10, 11, 12, 13, 14, 15, 16, 17, 18, 19, 20, 21, 22, 23, 24, 25, 26, 27 or 28 days. In certain embodiments, the cryopreserved cells are thawed after being cryopreserved for about 1, 2, 3, 4, 5, 6, 7, 8, 9, 10, 11, 12, 13, 14, 15, 16, 17, 18, 19, 20, 21, 22, 23, 24, 25, 26, 27 or 28 months. In certain embodiments, the cryopreserved cells are thawed after being cryopreserved for about 1, 2, 3, 4, 5, 6, 7, 8, 9 or 10 years.

Suitable thawing medium includes, but is not limited to, normal saline, plasmalyte culture medium including, for example, growth medium, eg., RPMI medium. In preferred embodiments, the thawing medium comprises one or more of medium supplements (e.g., nutrients, cytokines and/or factors) . Medium supplements suitable for thawing cells provided herein include, for example without limitation, serum such as human serum AB, fetal bovine serum (FBS) or fetal calf serum (FCS), vitamins, human serum albumin (HSA), bovine serum albumin (BSA), amino acids (eg., L-glutamine), fatty acids (e.g., oleic acid, linoleic acid or palmitic acid), insulin (e.g., recombinant human insulin), transferrin (iron saturated human transferrin), 0-mercaptoethanol, stem cell factor (SCF), Fms-like-tyrosine kinase 3 ligand Flt3-I..), cytokines such as interleukin-2 (IL-2), interleukin-7 (IL-7), interleukin-15 (IL-15), thrombopoietin (Tpo) or heparin. In a specific embodiment, the thawing medium useful in the methods provided herein comprises RPMI. In another specific embodiment, said thawing medium comprises plasmalyte. In another specific embodiment, said thawing medium comprises about 0.5-20% FBS. In another specific embodiment, said thawing medium comprises about 1, 2, 5, 10, 15 or 20% HBS. In another specific embodiment, said thawing medium comprises about 0.5%-20% HSA. In another specific embodiment, said thawing medium comprises about 1, 2.5, 5, 10, 15, or 20% HSA. In a more specific embodiment, said thawing medium comprises RPMI and about 10% FBS In another more specific embodiment, said thawing medium comprises plasmalyte and about 5% EdSA.

The cryopreservation methods provided herein can be optimized to allow for long-term storage, or under conditions that inhibit cell death by, eg, apoptosis or necrosis. In one embodiments, the post-thaw cells comprise greater than about 60%, 65%, 70%, 75%, 80%, 85%, 90%, 95% or 98% of viable cells, as determined by, e.g., automatic cell counter or trypan blue method. In another embodiment, the post-thaw cells comprise about 0.5, 1, 5, 10, 15, 20 or 25% of dead cells. In another embodiment, the post-thaw cells comprise about 0.5, 1, 5, 10, 15, 20 or 25% of early apoptotic cells. In another embodiment, about 0.5, 1, 5, 10, 15 or 20% of post-thaw cells undergo apoptosis after 1, 2, 3, 4, 5, 6, 7, 8, 9, 10, 11, 12, 13, 14, 15, 16, 17, 18, 19, 20, 21, 22, 23, 24, 25, 26, 27 or 28 days after being thawed, e.g., as determined by an apoptosis assay (e.g., TO-PRO3 or AnnV/131 Apoptosis assay kit). In certain embodiments, the post-thaw cells are re-cryopreserved after being cultured, expanded or differentiated using methods provided herein.

### Uses of Engineered NK Cells

The engineered NK cells provided herein can be used in methods of treating individuals having cancer, eg., individuals having solid tumor cells and/or blood cancer cells. The engineered NK cells provided herein can also be used in methods of suppressing proliferation of tumor cells.

### Treatment of Subjects Having Cancer

In one embodiment, provided herein is a method of treating an individual having a cancer, for example, a brain cancer, a blood cancer or a solid tumor, comprising administering to said individual a therapeutically effective amount of engineered NK cells. In certain embodiments, the individual has a deficiency of natural killer cells, e.g., a deficiency of NK cells active against the individual’s cancer. As used herein, an “effective amount” is an amount that, e.g., results in a detectable improvement of, lessening of the progression of, or elimination of, one or more symptoms of a cancer from which the individual suffers.

In another embodiment, provided herein is a method of suppressing the proliferation of tumor cells comprising contacting the tumor cells with a therapeutically effective amount of engineered NK cells disclosed herein.

In another specific embodiment, the method further comprises contacting the tumor cells with an effective amount of an anticancer compound. In some embodiments, the brain cancer is a neuroblastoma.

The invention includes a method to treat a patient with a tumor, typically a human, by administering an effective amount of an engineered NK-cell composition described herein.

The dose administered may vary. In some embodiments, the engineered NK-cell composition is administered to a patient, such as a human in a dose ranging from 1 × 10⁶ cells/m² to 1 × 10⁸ cells/m². The dose can be a single dose, or multiple separate doses. In some embodiments, the engineered NK-cell composition dosage is about any of the following values: 1 × 10⁶ cells/m², 2 × 10⁶ cells/m², 3 × 10⁶ cells/m², 4 × 10⁶ cells/m², 5 × 10⁶ cells/m², 6 × 10⁶ cells/m², 7 × 10⁶ cells/m², 8 × l0⁴ cells/m², 9 × 10⁶ cells/m², 1 × 10⁷ cells/m², 2 × 10⁷ cells/m², 3 × 10⁷ cells/m², 4 × 10⁷ cells/m², 5 × 10⁷ cells/m², 6 × 10⁷ cells/m², 7 × 10⁷ cells/m², 8 × 10⁷ cells/m², 9 × 10⁷ cells/m², or 1 × 10⁸ cells/m².

The engineered NK-cell composition may be administered by any suitable method. In some embodiments, the engineered NK-cell composition is administered to a patient, such as a human as an infusion and in a particular embodiment, an infusion with a total volume of 1 to 10 cc. In some embodiments, the engineered NK-cell composition is administered to a patient as a 1, 2, 3, 4, 5, 6, 7, 8, 9, or 10 cc infusion. In some embodiments, the engineered NK-cell composition when present as an infusion is administered to a patient over 10, 20, 30, 40, 50, 60 or more minutes to the patient in need thereof.

In one embodiment, a patient receiving an infusion has vital signs monitored before, during, and 1-hour post infusion of the engineered NK-cell composition. In certain embodiments, patients with stable disease (SD), partial response (PR), or complete response (CR) up to 6 weeks after initial infusion may be eligible to receive additional infusions, for example, 1, 2, 3, 4, 5, 6, 7, 8, 9 or 10 additional infusions several weeks apart, for example, up to about 2, 3, 4, 5, 6, 7, 8, 9 or 10 weeks apart

### Hematological and Solid Tumors Targeted for Treatment

The engineered NK-cell compositions described herein can be used to treat a patient with a solid or hematological tumor.

Neuroblastoma, along with many other solid tumors, evades immune detection by sequestering itself in a suppressive milieu, dominated largely by the cytokine transforming growth factor beta (TGFp). Reports suggest that immune efficacy in solid tumor patients is hampered by the TGFfl-rich tumor microenvironment.¹⁻⁶ Anti-tumor activity of effector cells in neuroblastoma patients has only been described in the specific setting of concurrent immune modulation, suggesting that the tumor microenvironment is largely to blame for this impaired functionality.⁷ Recent years have seen a renewed optimism for immunotherapy, specifically in adoptive cell therapies, which involves the treatment of cancer patients with autologous or allogeneic transplanted immune cells.⁸⁻¹³ Adoptive cell therapies offer the potential for personalized therapeutic treatment options with verified clinical efficacy, which could yield better outcomes in neuroblastoma. In certain embodiments, the tumor is a neuroblastoma.

In certain other embodiments, the individual having a cancer has been treated with at least one anticancer drug, and has relapsed, prior to said administering.

Lymphoid neoplasms are broadly categorized into precursor lymphoid neoplasms and mature ‘1′-cell, B-cell or natural killer cell (NK) neoplasms. Chronic leukemias are those likely to exhibit primary manifestations in blood and bone marrow, whereas lymphomas are typically found in extramedullary sites, with secondary events in the blood or bone. Over 79,000 new cases of lymphoma were estimated in 2013. Lymphoma is a cancer of lymphocytes, which are a type of white blood cell. Lymphomas are categorized as f-todgkin’s or non-Hodgkin’s. Over 48,000 new cases of leukemias were expected in 2013.

In one embodiment, the disease or disorder is a hematological malignancy selected from a group consisting of leukemia, lymphoma and multiple myeloma.

In one embodiment, the methods described herein can be used to treat a leukemia. For example, the patient such as a human may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AM!.): large granular lymphocytic leukemia; or Adult T-cell chronic leukemia. In one embodiment, the patient suffers from an acute myelogenous leukemia, for example an undifferentiated AML (M0); myeloblastic leukemia (Ml; with/without minimal cell maturation); myeloblastic leukemia (M2; with cell maturation); promyelocytic leukemia (M3 or M3) variant [M3V]); myelomonocytic leukemia (M4 or M4 variant with eosinophilia (M.4EJ); monocytic leukemia (M5); erythroleukemia (M6); or megakaryoblastic leukemia (11iT7).

In a particular embodiment, the hematological malignancy is a lymphoma or lymphocytic or myelocytic proliferation disorder or abnormality. In one embodiment, the lymphoma is a non-Hodgkin’s lymphoma. In one embodiment, the lymphoma is a Hodgkin’s lymphoma.

In some aspects, the methods described herein can be used to treat a patient such as a human, with a Non-Hodgkin’s Lymphoma such as, but not limited to: an AIDS-Related Lymphoma; Anaplastic Large-Cell Lymphoma; Angioimmunoblastic Lymphoma; Blastic NK-Cell Lymphoma; Burkitt’s Lymphoma; Burkitt-like Lymphoma (Small Non-Cleaved Cell Lymphoma); Chronic Lymphocytic Leul:emiaJSn3al1 Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta ‘1′-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom’s Macroglobulinemia.

Alternatively, the methods described herein can be used to treat a patient, such as a human, with a Hodgkin’s Lymphoma, such as, but not limited to: Nodular Sclerosis Classical Hodgkin’s Lymphoma (CHL); Mixed Cellularity CHI..; Lymphocyte-depletion CHL; Lymphocyte-rich t:HLL; Lymphocyte Predominant Hodgkin Lymphoma; or Nodular Lymphocyte Predominant HL..

Alternatively, the methods described herein can be used to treat a patient, for example a human, with specific B-cell lymphoma or proliferative disorder such as, but not limited to: multiple myeloma, Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

Abnormal proliferation of T cells, B cells, and/or NK cells can result in a wide range of cancers. A host, for example a human, afflicted with any of these disorders can be treated with an effective amount of the TAA-L composition as described herein to achieve a decrease in symptoms (a palliative agent) or a decrease in the underlying disease (a disease modifying agent).

Alternatively, the methods described herein can be used to treat a patient, such as a human, with a hematological malignancy, for example but not limited to T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-typeT-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/ lymphoma; Enteropathy-associated T-cell lymphoma; Hepatosplenic T-cell lymphoma; or Subcutaneous panniculitis-like T-cell lymphoma

In one embodiment, the engineered NK-cell compositions disclosed herein is used to treat a patient with a selected hematopoietic malignancy either before or after hematopoietic stem cell transplantation (HSCT). In some embodiments, the composition is used to treat a patient with a sbrain cancer. In one embodiment, the composition is used to treat a patient with a selected hematopoietic malignancy up to about 30, 35, 40, 45, or 50 days after HSCT. In one embodiment, the composition is used to treat a patient with a selected hematopoietic malignancy after neutrophil engraftment. In some embodiments, the composition is used to treat a patient with a selected hematopoietic malignancy before HSCT, such as one week, two weeks, three weeks or more before HSCT. In some embodiments, the composition is used to treat a patient with a brain malignancy, such as neuroblastoma.

In some aspects, the tumor is a solid tumor. In one embodiment, the solid tumor is Wilms Tumor. In one embodiment, the solid tumor is osteosarcoma. In one embodiment, the solid tumor is Ewing sarcoma. In one embodiment, the solid tumor is neuroblastoma. In one embodiment, the solid tumor is soft tissue sarcoma. In one embodiment, the solid tumor is rhabdomyosarcoma.

Non-limiting examples of tumors that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast, triple negative breast cancer, HER2-negative breast cancer, HER2-positive breast cancer, male breast cancer, late-line metastatic breast cancer, progesterone receptor-negative breast cancer, progesterone receptor-positive breast cancer, recurrent breast cancer), brain cancer (e.g., meningioma; glioma, e.g, astrocytoma, oligodendroglioma, medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi’s sarcoma, multiple idiopathic hemorrhagic sarcoma), endometrial cancer (e.g., uterine cancer, uterine sarcoma), esophageal cancer (e.g., adenocarcinoma of the esophagus, Barrett’s adenocarcinoma), Ewing’s sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms’ tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget’s disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g, squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget’s disease of the vulva).

### Treatment of Subjects Having a Viral Infection

In another embodiment, provided herein is a method of treating an individual having a viral infection, comprising administering to said individual a therapeutically effective amount of engineered NK cells as described herein. In certain embodiments, the individual has a deficiency of natural killer cells, e.g., a deficiency of NK cells active against the individual’s viral infection. In certain embodiments, the therapeutically effective amount is an amount that, e.g., results in a detectable improvement of, lessening of the progression of, or elimination of, one or more symptoms of said viral infection. In specific embodiments, the viral infection is an infection by a virus of the Adenoviridae, Picornaviridae, Herpesviridae, Hepadnaviridae, Flaviviridae, Retroviridae, Orthomyxoviridae, Paramyxoviridae, Papilommaviridae, Rhabdoviridae, or Togaviridae family. In more specific embodiments, said virus is human immunodeficiency virus (HIV) coxsackievirus, hepatitis A virus (HAV), poliovirus, Epstein-Barr virus (EBV), herpes simplex type 1 (HSV1), herpes simplex type 2 (HSV2), human cytomegalovirus (CMV), human herpesvirus type 8 (HHV8), herpes zoster virus (varicella zoster virus (VZV) or shingles virus), hepatitis B virus (HBV), hepatitis C virus (HCV), hepatitis D virus (HDV), hepatitis E virus (HEV), influenza virus (e.g., influenza A virus, influenza B virus, influenza C virus, or thogotovirus), measles virus, mumps virus, parainfluenza virus, papillomavirus, rabies virus, or rubella virus.

In other more specific embodiments, said virus is adenovirus species A, serotype 12, 18, or 31; adenovirus species B, serotype 3, 7, 11, 14, 16, 34, 35, or 50; adenovirus species C, serotype 1, 2, 5, or 6; species D, serotype 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, or 51; species E, serotype 4; or species F, serotype 40 or 41.

In certain other more specific embodiments, the virus is Apoi virus (APOIV), Aroa virus (AROAV), bagaza virus (BAGV), Banzi virus (BANV), Bouboui virus (BOUV), Cacipacore virus (CPCV), Carey Island virus (CIV), Cowbone Ridge virus (CRV), Dengue virus (DENV), Edge Hill virus (EHV), Gadgets Gully virus (GGYV), Ilheus virus (ILHV), Israel turkey meningoencephalomyclitis virus (ITV), Japanese encephalitis virus (JEV), Jugra virus (JUGV), Jutiapa virus (JUTV), kadam virus (KADV), Kedougou virus (KEDV), Kokobera virus (KOKV), Koutango virus (KOUV), Kyasanur Forest disease virus (KFDV), Langat virus (LGTV), Meaban virus (MEAV), Modoc virus (MODV), Montana myotis leukoencephalitis virus (MMLV), Murray Valley encephalitis virus (MVEV), Ntaya virus (NTAV), Omsk hemorrhagic fever virus (OHFV), Powassan virus (POWV), Rio Bravo virus (RBV), Royal Farm virus (RFV), Saboya virus (SABV), St. Louis encephalitis virus (SLEV), Sal Vieja virus (SVV), San Perlita virus (SPV), Saumarez Reef virus (SREV), Sepik virus (SEPV), Tembusu virus (TMUV), tick-borne encephalitis virus (TBEV), Tyuleniy virus (TYUV), Uganda S virus (UGSV), Usutu virus (USUV), Wesselsbron virus (WESSV), West Nile virus (WNV), Yaounde virus (YAOV), Yellow fever virus (YFV), Yokose virus (YOKV), or Zika virus (ZIKV).

In other embodiments, the engineered NK cells are administered to an individual having a viral infection as part of an antiviral therapy regimen that includes one or more other antiviral agents. Specific antiviral agents that may be administered to an individual having a viral infection include, but are not limited to: imiquimod, podofilox, podophyllin, interferon alpha (IFNα), reticolos, nonoxynol-9, acyclovir, famciclovir, valaciclovir, ganciclovir, cidofovir, amantadine, rimantadine, ribavirin; zanamavir and oseltaumavir; protease inhibitors such as indinavir, nelfinavir, ritonavir, or saquinavir; nucleoside reverse transcriptase inhibitors such as didanosine, lamivudine, stavudine, zalcitabine, or zidovudine; and non-nucleoside reverse transcriptase inhibitors such as nevirapine, or efavirenz.

### Administration of Engineered NK-Cell Compositions

Methods for administration of cells for adoptive cell therapy are known and may be used in connection with the provided methods and the engineered NK-cell compositions. For example, adoptive T-cell therapy methods are described, e.g., in U.S. Pat. Application Publication No. 2003/0170238 to Gruenberg et al; U.S. Pat No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The administration of the engineered NK-cell composition may vary. In one aspect, the engineered NK-cell composition may be administered to a patient such as a human at an interval selected from once every 1 week, 2 weeks, 3 weeks, 4 weeks, 5 weeks, 6 weeks, 7 weeks, 8 weeks, or more after the initial administration of the engineered NK-cell composition. In a typical embodiment, the engineered NK-cell composition is administered in an initial dose then at every 4 weeks thereafter. In one embodiment, the engineered NK-cell composition may be administered repetitively to 1, 2, 3, 4, 5, 6, or more times after the initial administration of the composition. In a typical embodiment, the engineered NK-cell composition is administered repetitively up to 10 more times after the initial administration of the engineered NK-cell composition. In an alternative embodiment, the engineered NK-cell composition is administered more than 10 times after the initial administration of the engineered NK-cell composition.

In some embodiments, the engineered NK-cell composition is administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the by the particular method used to administer the cell composition. Accordingly, there are a variety of suitable formulations For example, the pharmaceutical composition can contain preservatives. Suitable preservatives may include, for example, methylparaben, propylparaben, sodium benzoate, and benzalkonium chloride. In some aspects, a mixture of two or more preservatives is used The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

In some embodiments, the pharmaceutical composition comprises the engineered NK-cell composition in an amount that is effective to treat or prevent the disease or condition, such as a therapeutically effective or prophylactically effective amount. Thus, in some embodiments, the methods of administration include administration of the engineered NK-cell composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the engineered NK-cell composition is administered at a desired dosage, which in some aspects includes a desired dose or number of cells and/or a desired ratio of T-cell subpopulations. Thus, the dosage of cells in some embodiments is based on a total number of cells (or number per m² or per kg body weight) and a desired ratio of the individual populations or sub-types. In some embodiments, the dosage of cells is based on a desired total number (or number per m² or per kg of body weight) of cells in the individual populations or of individual cell types. In some embodiments, the dosage is based on a combination of such features, such as a desired number of total cells, desired ratio, and desired total number of cells in the individual populations.

In some embodiments, the engineered NK-cell composition is administered at or within a tolerated difference of a desired dose of total cells, such as a desired dose of T cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, eg., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual T-cell subpopulations. Thus, in some embodiments, the dosage is based on a desired fixed or minimum dose of T-cell subpopulations and a desired ratio thereof.

In certain embodiments, engineered NK-cell composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 10⁴ and at or about 10⁹ cells/meter² (m²) body surface area, such as between 10⁵ and 10⁶ cells/m² body surface area, for example, at or about 1×10⁵ cells/ m², 1.5×10⁵ cells/m², 2×10⁵ cells/ m², or 1×10⁶ cells/m² body surface area. For example, in some embodiments, the cells are administered at, or within a certain range of error of, between at or about 10⁴ and at or about 10⁹ T cells/meter² (m²) body surface area, such as between 10⁵ and 10⁶ T cells/ m² body surface area, for example, at or about 1×10⁵ T cells/m², 1.5×10⁵ T cells/m², 2×10⁵ T cells/m², or 1×10⁶ T cells/m² body surface area.

In some embodiments, the cells are administered at or within a certain range of error of between at or about 10⁴ and at or about 10⁹ cells/meter² (m²) body weight, such as between 10⁵ and 10⁶ cells/ m² body weight, for example, at or about 1×10⁵ cells/m², 1.5×10⁵ cells/m², 2×10⁵ cells/kg, or 1×10⁶ cells/m² body surface area.

### Product Release Testing and Characterization

Prior to infusion, the engineered NK-cell composition may be characterized for safety and release testing Product release testing, also known as lot or batch release testing, is an important step in the quality control process of drug substances and drug products. This testing verifies that an engineered NK-cell composition meets a pre-determined set of specifications. Pre-determined release specifications for engineered NK-cell compositions include confirmation that the cell product is >70% viable, has <5.0 EU/ml of endotoxin, is negative for aerobic, anaerobic, fungal pathogens and mycoplasma, and lacks reactivity to allogeneic PHA blasts, for example, with less than 10% lysis to PHA blasts The HLA identity between the engineered NK-cell composition and the donor is also confirmed.

### Monitoring

Following administration of the cells, the biological activity of the administered cell populations in some embodiments is measured, e.g., by any of a number of known methods. In certain embodiments, the ability of the administered cells to destroy target cells can be measured using any suitable method known in the art, such as cytotoxicity assays described in, for example, Kochenderfer et al., J. Immunotherapy, 32(7): 689-702 (2009), and Herman et al. J Immunological Methods, 285(1): 25-40 (2004), all incorporated herein by reference. In certain embodiments, ⁵¹chromium release assay is used for measuring NK-cell activity. In other embodiments, flow cytometry-based NK-cell cytotoxicity assay (FCA) may be used. FCA has several advantages such as discrimination of target cells from effector cells and of dead ones from live target cells, enumeration of NK-cell subsets, and possibility of large number of tests. FCA can also measure the specific NK-cell activation markers in addition to the analysis of target cells cytotoxicity. Since strong correlation between CD107a surface expression and NK-cell cytotoxicity was reported (Cellular Immunology. 2009;254(2): 149-154; Journal of Immunological Methods. 2011;372(1-2):187-195) and NK-cell function was influenced by cytokine secretion, simultaneous assessment of CD107a and cytokine/chemokine production could be helpful for the complete analysis of NK-cell function. The real-time cell electronic sensing (RT-CES) system using xCELLigence (Roche Diagnostics, Penzberg, Germany) may also be used as an alternative for label-free in vitro quantification of NK-cell mediated cytotoxicity (Journal of Immunological Methods. 2006;309(1-2):25-33). The RT-CES system is microelectronic sensor-based platform integrated into the bottom of microtiter plates, which measure any changes to the cell number, size, morphology, or attachment quality of adherent cells in real time. If target cells are adhered to the culture plate bottom that is coated with the gold microelectrodes, the electrical impedance occurs and is converted to the cell index. In NK function test, when effector cells are added to growing adherent target cells, the cell index decreases and can be changed into the NK-cell cytotoxicity, as it has been used previously for cytotoxic function of NK cell lines on several tumor cell lines.

### Combination Therapies

In one aspect of the invention, the compositions disclosed herein can be beneficially administered in combination with another therapeutic regimen for beneficial, additive, or synergistic effects.

In one embodiment, the composition is administered in combination with another therapy in the same or second compositrion. In some embodiments, the combined therapy is administered to treat a solid tumor The second therapy can be a pharmaceutical or a biologic agent (for example an antibody) to increase the efficacy of treatment with a combined or synergistic approach.

Treatment of an individual having cancer using the engineered NK cells described herein can be part of an anticancer therapy regimen that includes one or more other anticancer agents. Such anti-cancer agents are well-known in the art. Specific anti-cancer agents that may be administered to an individual having cancer, e.g., an individual having tumor cells, in addition to the engineered NK cells, and optionally perfusate, perfusate cells, natural killer cells other than the engineered NK cells, include, but are not limited to: acivicin; aclarubicin; acodazole hydrochloride; acronine; adozelesin; adrucil; aldesleukin; altretamine; ambomycin; ametantrone acetate; amsacrine; anastrozole, anthramycin; asparaginase, asperlin; avastin (bevacizumab); azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer, carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel: doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride, estramustine; estramustine phosphate sodium; etanidazole; etoposide; etoposide phosphate; etoprine; fadrozole hydrochloride; fazarabine; fenretinide; floxuridine, fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin: mitomalcin; mitomycin; mitosper; mitotane; mitoxantrone hydrochloride; mycophenolic acid; nocodazole, nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin, plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin: streptonigrin; streptozocin; sulofenur, talisomycin; tecogalan sodium; taxotere; tegafur, teloxantrone hydrochloride; temoporfin; teniposide; teroxirone; testolactone; thiamiprine; thioguanine; thiotepa; tiazofurin; tirapazamine; toremifene citrate, trestolone acetate; triciribine phosphate; trimetrexate; trimetrexate glucuronate; triptorelin; tubulozole hydrochloride; uracil mustard; uredepa; vapreotide; verteportin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretaniine; ambamustine; amidox; amifostine; aminolevulinic acid; amrubicin, amsacrine; anagrelide; anastrozole, andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators, apoptosis regulators; apurinic acid; ara-CDP-DL-PTBA; arginine deaminase; asulacrine; atamestane; atrimustine; axinastatin 1; axinastatin 2; axinastatin 3; azasctron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor, bicalutamide; bisantrene; bisaziridinylspermine; bisnafide; bistratene A; bizelesin; breflate; bropirimine; budotitane; buthionine sulfoximine; calcipotriol; calphostin C; camptosar (also called Campto; irinotecan) camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B-, cetrorelix; chlorlns; chloroquinoxaline sulfonamide: cicaprost; cis-porphyrin; cladribine; clomifene analogues; clotrimazole; collismycin A; collismycin B; combretastatin A4; combretastatin analogue; conagenin, crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemnin B; didox; diethylnorspennine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; etlornithine; elemene; emitefur, epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone, fludarabine; fluorodaunorunicin hydrochloride; forfenimex; formestane; fostriecin; fotemustine; gadolinium texaphyrin; gallium nitrate; galocitabine; ganirelix; gelatinase inhibitors, gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomobalicondrin B; itasetron; jasplalrinolide: kahalalide F; lamellarin-N triacetate; lanreotide; leinamycin; lenograstirn; lentinan sulfate; leptolstatin; letrozole; leukemia inhibiting factor; leukocyte alpha interferon; leuprolide=estrogen=progesterone;leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone;loxoribine; lurtotecan; lutetium texaphyrin; lysofylline; lytic peptides; maitansine; mannostatin A; marmastat, masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol: mitomycin analogues; mitonafide; mitotoxin fibroblast growth factor-saporin; mitoxantrone; mofarotene; molgramostim; Erbitux (cetuximab), human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide, nisamycin; nitric oxide modulators; nitroxide antioxidant; nitrullyn; oblimersen (GENASENSE®); O6-benzylguanine; octreotide; okicenone; oligonucleotides; onapristone; ondansetron; ondansetron; oracin; oral cytokine inducer; ormaplatin; osatcrone; oxaliplatin (e.g., Floxatin); oxaunomycin; paclitaxel; paclitaxel analogues; paclitaxel derivatives; palauamine; palmitoylrhizoxin; pamidronic acid; panaxytriol; panomifene; parabactin: pazelliptine; pegaspargase; peldesine; pentosan polysulfate sodium; pentostatin; pentrozole; perflubron; perfosfamide, perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator, protein kinase C: inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron: ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; satingal; saintopin; SarCNU; sarcophytol A; sargramostim; Sdi 1 mimetics; semustine; senescence derived inhibitor 1, sense oligonucleotides, signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium, telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; Vectibix (panitumumab)velaresol; veramine; verdins; verteporfin; vinorelbine; vinxaltine; vitaxin; vorozole; Welcovorin (leucovorin); Xeloda (capecitabine); zanoterotie; zeniplatin; zilascorb; and zinostatin stimalamer.

In one embodiment, the additional therapy is a monoclonal antibody (MAb). Some MAbs stimulate an immune response that destroys tumor cells. Similar to the antibodies produced naturally by B cells, these MAbs “coat” the tumor cell surface, triggering its destruction by the immune system. FDA-approved MAbs of this type include rituximab, which targets the CD20 antigen found on non-Hodgkin lymphoma cells, and alemtuzumab, which targets the CD52 antigen found on B-cell chronic lymphocyticieukemia (CLL) cells. Rituximab may also trigger cell death (apoptosis) directly. Another group of MAbs stimulates an antitumor immune response by binding to receptors on the surface of immune cells and inhibiting signals that prevent immune cells from attacking the body’s own tissues, including tumor cells. Other MAbs interfere with the action of proteins that are necessary for tumor growth. For example, bevacizumab targets vascular endothelial growth factor (VEGF), a protein secreted by tumor cells and other cells in the tumor’s microenvironment that promotes the development of tumor blood vessels. When bound to bevacizumab, YEGF cannot interact with its cellular receptor, preventing the signaling that leads to the growth of new blood vessels. Similarly, cetuximab and panitumumab target the epidermal growth factor receptor (EGFR). MAbs that bind to cell surface growth factor receptors prevent the targeted receptors from sending their normal growth-promoting signals. They may also trigger apoptosis and activate the immune system to destroy tumor cells. Another group of tumor therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a tumor cell, and the cell-killing substance is taken up by the cell. FDA-approved conjugated MAbs that work this way include 90Y- ibritumomab tiuxetan, which targets the CD20 antigen to deliver radioactive yttrium-90 to B-cell non-Hodgkin lymphoma cells; ¹³¹I-tositumomab, which targets the CD20 antigen to deliver radioactive ¹³¹I to non-Hodgkin lymphoma cells.

In one embodiment, the additional agent is a cytokine, for example, but not limited to IL-2, IL-15, IL-12, IL-18, IL-21. Such agent can either be administer separately or secreted by cellular product including the engineered NK cells described herein.

In one embodiment, the additional agent is a modified T- cell or NK-cell. Such agent can either be administer separately or the modified T- cell or NK-cell is expressed by the engineered NK cells described herein.

In one embodiment, the additional agent is an immune checkpoint inhibitor (ICI), for example, but not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combinations thereof

In one embodiment, the immune checkpoint inhibitor is a PD-1 inhibitor that blocks the interaction of PD-1 and PD-L1 by binding to the PD-1 receptor, and in turn inhibits immune suppression. In one embodiment, the immune checkpoint inhibitor is a PD-1immune checkpoint inhibitor selected from nivolumab (Opdivo®), pembrolizumab (Keytruda®), pidiliztimab, AMP-224 (AstraZeneca and Medlmmune), PF-06801591 (Pfizer), MED10680(AstraZeneca), PDR001 (Novartis), REGN2810 (Regeneron), MGA012 (MacroGenics), BCYB-A317 (BeiGene) SHR-12-1 (Jiangsu Hengrui Medicine Company and Incyte Corporation), TSR-042 (Tesaro), and the PD-L1/VISTA inhibitor CA-17U (Curls Inc ).

In one embodiment, the immune checkpoint inhibitor is the PD-1 immune checkpoint inhibitor nivolumab (Opdivo®) administered in an effective amount for the treatment of Hodgkin’s lymphoma. In another aspect of this embodiment, the immune checkpoint inhibitor is the PD-1 immune checkpoint inhibitor pembrolizumab (Keytruda®) administered in an effective amount. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL).

In one embodiment, the immune checkpoint inhibitor is a PD-L1 inhibitor that blocks the interaction of PD-1 and PD-L1 by binding to the PD-L1 receptor, and in turn inhibits immune suppression. PD-L1inhibitors include, but are not limited to, atezolizumab, durvalumab, KN035CA-170 (Curis Inc.), and LY3300054 (Eli Lilly).

In one embodiment, the immune checkpoint inhibitor is the PD-L1 immune checkpoint inhibitor atezolizumab (Tecentriq®) administered in an effective amount. In another aspect of this embodiment the immune checkpoint inhibitor is durvalumab (AstraZeneca and Medlmmune) administered in an effective In yet another aspect of the embodiment, the immune checkpoint inhibitor is KN035 (Alphamab). An additional example of a PD-L1 immune checkpoint inhibitor is BMS-936559 (Bristol-Myers Squibb), although clinical trials with this inhibitor have been suspended as of 2015.

In one aspect of this embodiment, the immune checkpoint inhibitor is a CTLA-4 immune checkpoint inhibitor that binds to CTLA-4 and inhibits immune suppression CTLA-4 inhibitors include, but are not limited to, ipilimumab, tremelimumab (AstraZeneca and Medlmmune), AGEN1884 and AGEN2041 (Agenus)

In one embodiment, the CTLA-4 immune checkpoint inhibitor is ipilimumab (Yervoy®) administered in an effective amount

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro).

Other immune checkpoint inhibitors for use in combination with the invention described herein include, but are not limited to, B7-H3/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 September 2; 5(9). pii: el2529 (DOI:10: 1371/journal.pone.0021146), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 Jan; 163(1): 77-87.

Current chemotherapeutic drugs that may be used in combination with the composition described herein include those used to treat cancer including cytarabine (cytosine arabinoside or ara-C) and the anthracycline drugs (such as daunorubicin/daunomycin, idarubicin, and mitoxantrone). Some of the other chemo drugs that may be used to treat AML include: Cladribine (Leustatin®, 2-CdA), Fludarabine (Fludara®), Topotecan, Etoposide (VP-16), 6-thioguanine (6-TG), Hydroxyurea (Hydrea®), Corticosteroid drugs, such as prednisone or dexamethasone (Decadron®). Methotrexate (MTX), 6-mercaptopurine (6-MP), Azacitidine (Vidaza®), Decitabine (Dacogen®) Additional drugs include dasatinib and checkpoint inhibitors such as novolumab, Pembrolizumab, and atezolizumab.

Current chemotherapeutic drugs that may be used in combination with the composition described herein include those used for lymphomas including: purine analogs such as fludarabine (Fludara®), pentostatin (Nipent®), and cladribine (2-CdA, Leustatin®), and alkylating agents, which include chlorambucil (LeukeranⓇ) and cyclophosphamide (Cytoxan®) and bendamustine (Treanda®). Other drugs sometimes used for CLL include doxorubicin (Adriamycin®), methotrexate, oxaliplatin, vincristine (Oncovin®), etoposide (VP-16), and cytarabine (ara-C:). Other drugs include Rituximab (Rituxan), Obinutuzumab (Gazyva™), Ofatumumab (Arzerra®), Alemtuzumab (Campath®) and Ibrutinib (Imbruvica™).

Current chemotherapeutic drugs that may be used in combination with the composition described herein include those used for CML including: Interferon, imatinib (Gleevec), the chemo drug hydroxyurea (Hydrea®), cytarabine (Ara-C), busulfan, cyclophosphamide (Cytoxan®), and vincristine (Oncovin®). Omacetaxine (Synribo®) is a chemo drug that was approved to treat CML that is resistant to some of the TKIs now in use.

Current chemotherapeutic drugs that may be used in combination with the composition described herein include those used for CMML, for example, Deferasirox (Exjade®), cytarabine with idarubicin. cytarabine with topotecan, and cytarabine with fludarabine, Hydroxyurea (hydroxycarbamate, Hydrea®), azacytidine (Vidaza®) and decitabine (Dacogen®).

Current chemotherapeutic drugs that may be used in combination with the composition described herein include those used for multiple myeloma include Pomalidomide (Pomalyst®), Carfilzomib (Kyprolis™), Everolimus (Afinitor®), dexamethasone (Decadron), prednisone and methylprednisolone (Solu-medrol®) and hydrocortisone.

Current chemotherapeutic drugs that may be used in combination with the composition described herein include those used for Hodgkin’s disease include Brentuximab vedotin (Adcetris™): anti-CD-30, Rituximab, Adriamycin® (doxorubicin), Bleomycin, Vinblastine, Dacarbazine (DTIC).

Current chemotherapeutic drugs that may be used in combination with the composition described herein include those used for Non-Hodgkin’s disease include Rituximab (Rituxan®), Ibritumomab (Zevalin®), tositumomab (Bexxar®), Alemtuzumab (Campath®) (CD52 antigen), Ofatumumab (Arzerra®), Brentuximab vedotin (Adcetris®) and Lenalidomide (Revlimid®).

Current chemotherapeutic drugs that may be used in combination with the composition described herein include those used for:
B-cell Lymphoma, for example:
Diffuse large B-cell lymphoma: CHOP (cyclophosphamide, doxorubicin, vincristine, and prednisone), plus the monoclonal antibody rituximab (Rituxan). This regimen, known as R-CHOP, is usually given for about 6 months.

Primary mediastinal B-cell lymphoma: R-CHOP.

Follicular lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Chronic lymphocytic leukemia/small lymphocytic lymphoma: R-CHOP.

Mantle cell lymphoma: fludarabine, cladribine, or pentostatin; bortezomib (Velcade) and lenalidomide (Revlimid) and ibrutinib (Imbruvica).

Extranodal marginal zone B-cell lymphoma ... mucosa-associated lymphoid tissue (MALT) lymphoma: rituximab; chlorambucil or fludarabine or combinations such as CVP, often along with rituximab.

Nodal marginal zone B-cell lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Splenic marginal zone B-cell lymphoma rituximab; patients with Hep C - anti-virals.

Burkitt lymphoma: methotrexate; hyper-CVAD - cyclophosphamide, vincristine, doxorubicin (also known as Adriamycin), and dexamethasone, Course B consists of methotrexate and cytarabine; CODOX-M - cyclophosphamide, doxorubicin, high-dose methotrexate/ifosfamide, etoposide, and high-dose cytarabine; etoposide, vincristine, doxorubicin, cyclophosphamide, and prednisone (EPOCH)

Lymphoplasmacytic lymphoma -rituximab.

Hairy cell leukemia - cladribine (2-CdA) or pentostatin; rituximab; interferon-alfa

T-cell lymphomas, for example:

Precursor T~lymphoblastic lymphoma/leukemia - cyclophosphamide, doxorubicin (Adriamycin), vincristine, L-asparaginase, methotrexate, prednisone, and, sometimes, cytarabine (ara-C). Because of the risk of spread to the brain and spinal cord, a chemo drug such as methotrexate is also given into the spinal fluid.

Skin lymphomas: Gemcitabine Liposomal doxorubicin (Doxil); Methotrexate; Chlorambucil; Cyclophosphamide, Pentostatin; Etoposide; Temozolomide; Pralatrexate; R-CHOP

Angioimmunoblastic T-cell lymphoma: prednisone or dexamethasone.

Extranodal natural killer/T-cell lymphoma, nasal type: CHOP.

Anaplastic large cell lymphoma: CHOP; pralatrexate (Folotyn), targeted drugs such as bortezomib (Velcade) or romidepsin (Istodax), or immunotherapy drugs such as alemtuzumab (Campath) and denileukin diftitox (Ontak).

Primary central nervous system (CNS) lymphoma - methotrexate; rituximab.

A more general list of suitable chemotherapeutic agents includes, but are not limited to, radioactive molecules, toxins, also referred to as cytotoxins or cytotoxic agents, which includes any agent that is detrimental to the viability of cells, agents, and liposomes or other vesicles containing chemotherapeutic compounds. Examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cisdichlorodiamine platinum (II) (DDP) cisplatin), diamino dichloro platinum, anthracyclines, antibiotics, antis, asparaginase, BCG live (intra-vesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens. Cyclophosphamide, Cyclothosphamide, Cytarabine, Cytarabine, cytochalasin B, Cytoxan, Dacarbazine, Dactinomycin, dactinomycin (formerly actinomycin), daunorubicin HCl, daunorucbicin citrate, denileukin diftitox, Dexrazoxane, Dibromomannitol, dihydroxy anthracin dione, Docetaxel, dolasetron mesylate, doxorubicin HCl, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCl, glucocorticoids, goserelin acetate, gramicidin D, granisetron HCl, hydroxyurea, idarubicin HCl, ifosfamide, interferon a-2b, irinotecan HCl, letrozole, leucovorin calcium, leuprolide acetate, levamisole HCl, lidocaine, lomustine, maytansinoid, mechlorethamine HCl, medroxyprogesterone acetate, megestrol acetate, melphalan HCl, mercaptipurine, mesna, methotrexate, methyhestosterone, mithramycin, mitomycin C, mitotane, mitoxantrone, nilutamide, octreotide acetate, ondansetron HCl, paclitaxel, pamidronate disodium, pentostatin, pilocarpine HCl, plimycin, polifeprosan 20 with carmustine implant, porfimer sodium, procaine, procarbazine HCl, propranolol, rituximab, sargramostim, streptozotocin, tamoxifen, taxol, teniposide, tenoposide, testolactone, tetracaine, thioepa chlorambucil, thioguanine, thiotepa, topotecan HCl, toremifene citrate, trastuzumab, tretinoin, valrubicin, vinblastine sulfate, vincristine sulfate, and vinorelbine tartrate.

Additional therapeutic agents that can be administered in combination with the compositions disclosed herein can include bevacizumab, sutinib, sorafenib, 2-methoxyestradiol, finasunate, vatalanib, vandetanib, aflibercept, volociximab, etaracizumab, cilengitide, erlotinib, cetuximab, panitumumab, gefitinib, trastuzumab, atacicept, rituximab, alemtuzumab, aldesleukine, atlizumab, tocilizumab, temsirolimus, everolimus, lucatumumab, dacetuzumab, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, oblimersen, plitidepsin, talmapimod, enzastaurin, tipifarnib, perifosine, imatinib, dasatinib, lenalidomide, thalidomide, simvastatin, and celecoxib.

In one aspect of the present invention, the compositions disclosed herein are administered in combination with at least one immunosuppressive agent. The immunosuppressive agent may be selected from the group consisting of a calcineurin inhibitor, e.g. a cyclosporin or an ascomycin, e.g Cyclosporin A (NEORAL®), tacrolimus, a mTOR inhibitor, e.g rapamycin or a derivative thereof, e.g. Sirolimus (RAPAMUNE®), Everolimus (Certican®), temsirolimus, biolimus-7, biolimus-9, a rapalog, e.g. azathioprine, campath 1H, a SIP receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, 15-deoxyspergualin, tresperimus, Leflunomide ARAVA®, anti-CD25, anti-IL2R, Basiliximab (SIMULECT®), Daclizumab (ZENAPAX®), mizorbine, methotrexate, dexamethasone, pimecrolimus (Elidel®), abatacept, belatacept, etanercept (Enbrel®), adalimumab (Humira®), infliximab (Remicade®), an anti-LFA-1 antibody, natalizumab (Antegren®), Enlimomab, ABX-CBL, antithymocyte immunoglobulin, siplizumab, and efalizumab.

In one aspect of the present invention, the engineered NK-cell composition described herein can be administered in combination with at least one anti-inflammatory agent. The anti-inflammatory agent can be a steroidal anti-inflammatory agent, a nonsteroidal anti-inflammatory agent, or a combination thereof In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In one aspect of the present invention, the engineered NK cell composition described herein can be administered in combination with at least one immune-modulatory agent.

All journal articles, patent applications and references from GenBank disclosed are incorporated by reference in their entireties.

### Methods of Manufacturing Engineered NK-Cell Compositions

### Collecting a Peripheral Blood Mononuclear Cell Product From a Donor

Isolation of PBMCs is well known in the art. Non-limiting exemplary methods of isolating PBMCs are provided in Grievink, H.W., et al. (2016) “Comparison of three isolation techniques for human peripheral blood mononuclear cells: Cell recovery and viability, population composition, and cell functionality,” Biopreservation and BioBanking, which is incorporated herein by reference. The PBMC product can be isolated from whole blood, an apheresis sample, a leukapheresis sample, or a bone marrow sample provided by a donor. In one embodiment, the starting material is an apheresis sample, which provides a large number of initially starting mononuclear cells, potentially allowing a large number of different T-cell subpopulations to be generated. In one embodiment, the PBMC product is isolated from a sample containing peripheral blood mononuclear cells (PBMCs) provided by a donor. In one embodiment, the donor is a healthy donor. In one embodiment, the PBMC product is derived from cord blood. In one embodiment, the donor is the same donor providing stem cells for a hematopoietic stem cell transplant (HSCT).

### Determining HLA Subtype

When the NK-cell subpopulations are generated from an allogeneic, healthy donor, the HLA subtype profile of the donor source is determined and characterized. Determining HLA subtype (i.e., typing the HLA loci) can be performed by any method known in the art. Non-limiting exemplary methods for determining HLA subtype can be found in Lange, V., et al., BMC Genomics (2014)15: 63; Erlich, H., Tissue Antigens (2012) 80:1-11; Bontadini, A., Methods (2012) 56:471-476; Dunn, P. P., Int J Immunogenet (2011) 38:463-473; and Hurley, C. K., “DNA-based typing of HLA for transplantation.” in Leffell, M. S., et al., eds., Handbook of Human Immunology, 1997 Boca Raton: CRC Press, each independently incorporated herein by reference. Preferably, the HLA-subtyping of each donor source is as complete as possible.

In one embodiment, the determined HLA subtypes include at least 4 HLA loci, preferably HLA-A, HLA-B, HLA-C, and HLA-DRB1. In one embodiment, the determined HLA subtypes include at least 6 HLA loci. In one embodiment, the determined HLA subtypes include at least 6 HLA loci. In one embodiment, the determined HLA subtypes include all of the known HLA loci. In general, typing more HLA loci is preferable for practicing the invention, since the more HLA loci that are typed, the more likely the allogeneic NK-cell subpopulations selected will have highest activity relative to other allogeneic NK-cell subpopulations that have HLA alleles or HLA allele combinations in common with the patient or the diseased cells in the patient

### Separating the Monocytes and the Lymphocytes of the Peripheral Blood Mononuclear Cell Product

In general, the PBMC product may be separated into various cell-types, for example, into platelets, red blood cells, lymphocytes, and monocytes, and the lymphocytes and monocytes retained for initial generation of the T-cell subpopulations The separation of PBMCs is known in the art. Non-limiting exemplary methods of separating monocytes and lymphocytes include Vissers et al., J Immunol Methods. 1988 Jun 13; 110(2):203-7 and Wahl et al , Current Protocols in Immunology (2005) 7.6A.1-7.6A.10, which are incorporated herein by reference. For example, the separation of the monocytes can occur by plate adherence, by CD14+ selection, or other known methods. The monocyte fraction is generally retained in order to generate dendritic cells used as an antigen presenting cell in the T-cell subpopulation manufacture. The lymphocyte fraction of the PBMC product can be cryopreserved until needed, for example, aliquots of the lymphocyte fraction (∼5×10⁷ cells) can be cryopreserved separately for both Phytohemagglutinin (PHA) Blast expansion and T-cell subpopulation generation.

### Generating Dendritic Cells

The generation of mature dendritic cells used for antigen presentation to prime T-cells is well known in the art. Non-limiting exemplary methods are included in Nair et al., “Isolation and generation of human dendritic cells.” Current protocols in immunology (2012) 0 7: Unit7.32. doi: 10.1002/0471142735.im0732s99 and Castiello et al., Cancer Immunol Immunother, 2011 Apr;60(4):457-66, which are incorporated herein by reference. For example, the monocyte fraction can be plated into a closed system bioreactor such as the Quantum Cell Expansion System, and the cells allowed to adhere for 2-4 hours at which point 1,000 U/mL of IL-4 and 800 U/mL GM-CSF can be added . The concentration of GM-CSF and IL-4 can be maintained. The dendritic cells can be matured using a cytokine cocktail. In one embodiment the cytokine cocktail consists of LPS (30 ng/mL), IL-4 (1,000 U/mL), GM-CSF (800 U/mL), TNF-Alpha (10 ng/mL), IL-6 (100 ng/mL), and IL-1beta (10 ng/mL). The dendritic cell maturation generally occurs in 2 to 5 days. In one embodiment, the adherent DCs are harvested and counted using a hemocytometer. In one embodiment, a portion of the DCs are cryopreserved for additional further stimulations.

### Pulsing the Dendritic Cell

The non-mature and mature dendritic cells are pulsed with one or more peptides, of a single TAA. For example, the dendritic cells can be pulsed using one or more peptides, for example specific epitopes and/or a pepmix. Methods of pulsing a dendritic cell with a TAA are known. For example, about 100 ng of one or more peptides of the TAA, for example a peptide library (PepMix), can be added per 10 million dendritic cells and incubated for about 30 to 120 minutes.

### Naive T-Cell Selection of Lymphocytes

In order to increase the potential number of specific TAA activated T-cells and reduce T-cells that target other antigens, it is preferable to utilize naive T-cells as a starting material. To isolate naive T-cells, the lymphocytes can undergo a selection, for example CD45RA+ cells selection. CD45RA+ cell selection methods are generally known in the art. Non-limiting exemplary methods are found in Richards et al , Immune memory in CD4+ CD45RA+ T cells. Immunology. 1997;91(3):331-339 and McBreen et al., J Virol. 2001 May; 75(9): 4091-4102, which are incorporated herein by reference. For example, to select for CD45RA⁺ cells, the cells can be labeled using I vial of CD45RA microbeads from Miltenyi Biotec per 1×10¹¹ cells after 5-30 minutes of incubation with 100 mL of CliniMACS buffer and approximately 3 mL of 10% human IVIG, 10 ug/mL DNAase I, and 200 mg/mL of magnesium chloride. After 30 minutes, cells will be washed sufficiently and resuspended in 20 mL of CliniMACS buffer. The bag will then be set up on the CLINIMACS Plus device and the selection program can be run according to manufacturer’s recommendations. After the program is completed, cells can be counted, washed and resuspended in “CTL Media” consisting of 44.5% EHAA Click’s, 44.5% Advanced RPMI, 10% Human Serum, and 1% GlutaMAX.

### Stimulating Naive T Cells With Peptide-Pulsed Dendritic Cell

Prior to stimulating naive T-cells with the dendritic cells, it may be preferable to irradiate the DCs, for example, at 25 Gy. The DCs and naive T-cells are then co-cultured. The naive T-cells can be co-cultured in a ratio range of DCs to T cells of about 1:5-1:50, for example, 1:5; 1:10, 1 :15, 1:20, 1:25, 1:30, 1:35, 1:40, 1:45, or about 1:50. The DCs and T-cells are generally co-cultured with cytokines. In one embodiment, the cytokines are selected from a group consisting of IL-6 (100 ng/mL), IL-7 (10 ng/mL), IL-15 (5 ng/mL), IL-12 (10 ng/mL), and IL-21 (10 ng/mL).

### EXAMPLES

### Example 1. Methods

The Examples described herein were performed with, but not limited to, the below methods.

### Experimental Design

The objective of in vitro assessments was to characterize the phenotype and function of transduced NK cells as compared to unmodified NK cells, and experiments were performed in duplicate or triplicate, with sample sizes identified in each corresponding figure . The objective of in vivo studies was to examine the effect of treatment with unmodified vs. transduced NK cells on tumor growth and animal survival, and experiments were performed with sample sizes identified in each corresponding figure. For all experimentation, NK cells were divided evenly into four or five groups before retroviral transduction (untransduced cells, mock-transduced, RBDNR, NKA, NKCT), and animals were randomly assigned to treatment groups.

### Cell Sources and Cell Lines

Umbilical cord blood mononuclear cells were harvested from fresh cord blood units obtained from MD Anderson Cancer Center under approved IRB protocols (Pro00003896) by density gradient separation, and NK cells were isolated by negative selection with the EasySep Human NK Cell Isolation Kit (Stem Cell Technologies, Vancouver, Canada). After 24 hours of activation with 10 ng/mL of human IL-15 (R&D Systems, Minneapolis, MN), NK cells were stimulated with K562 feeder cells, modified to express membrane-bound IL-15 and 41BBL^{21.48} (generously obtained from Baylor College of Medicine (Pro00003869)), which were irradiated at 200 Gy and cultured with NK cells at a 2:1 K562:NK cell ratio. NK cells^{37,66.67} were cultured in Stem Cell Growth Medium (CellGenix, Germany) supplemented with 200 IU/mL human IL-2, 15 ng/mL human IL-15, 10% Heat Inactivated FBS (Gibco, Thermo Fisher Scientific, Waltham, MA), and 1% (Glutamax (Gibco, Thermo Fisher Scientific, Waltham, MA). Modified and unmodified K562 cell lines were cultured with IMDM (Thermo Fisher Scientific, Waltham, MA) supplemented with 10% Heat Inactivated FBS (Gibco, Thermo Fisher Scientific, Waltham, MA), 1% Penicillin-Streptomycin, and 1% Glutamax (Gibco, Thermo Fisher Scientific, Waltham, MA). Neuroblastoma line SHSY5Y was purchased from ATCC (Manassas, VA) and grown in a 1:1 medium of DMEM and F12K medium supplemented with 10% Heat Inactivated FBS (Gibco, Thermo Fisher Scientific, Waltham, MA), and 1% Glutamax (Gibco, Thermo Fisher Scientific, Waltham, MA). We performed HLA and STR profiling to verify the identify and type of the SHSY5Y tumor line (Genetica Cell Line Testing, Burlington, NC). For generating the bioluminescent neuroblastoma line used in vivo, SHSY5Y was transduced with 2.5×10⁶ CFU of CMV-Firefly-luciferase-puro-resistant (Cellomics Technology, Halethorpe, MD) as per manufacturer’s protocol. Bioluminescence was assessed with the Pierce Luciferase Dual Assay Kit (Thermo Fisher Scientific, Waltham, MA) and positive clones isolated by puromycin-resistance and expanded for use, and the cell line was identified as SHSY5Y-luc.

### Generation of Plasmids and Retrovirus Production

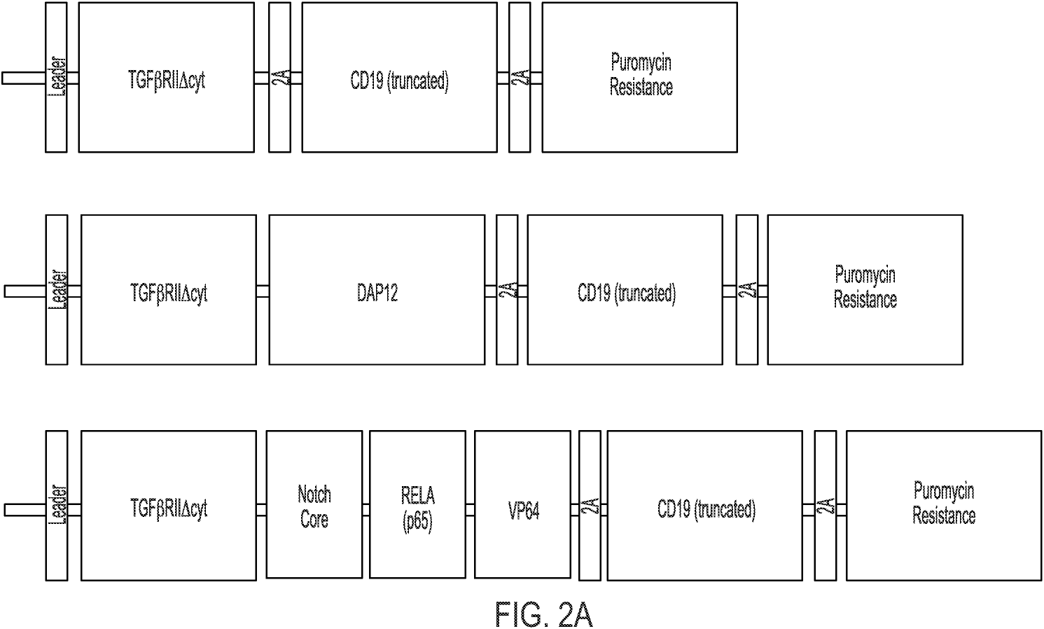

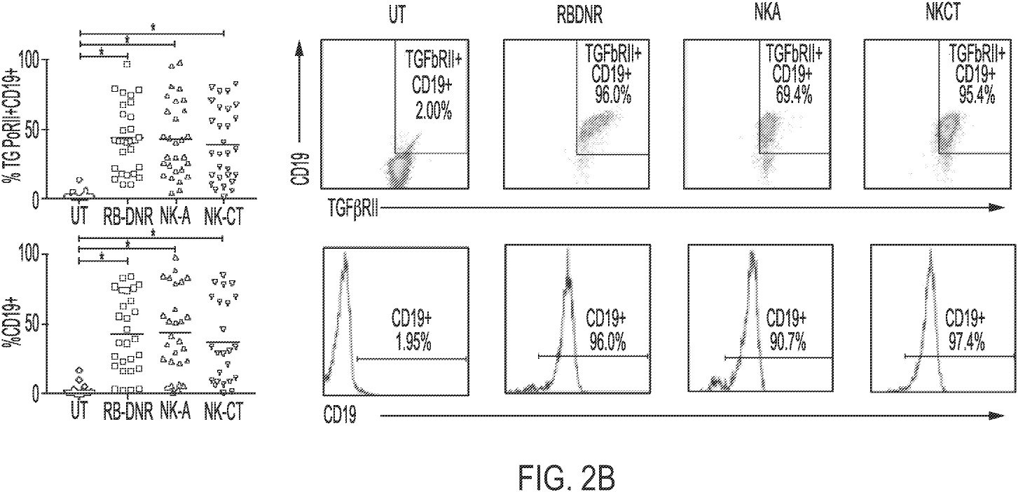

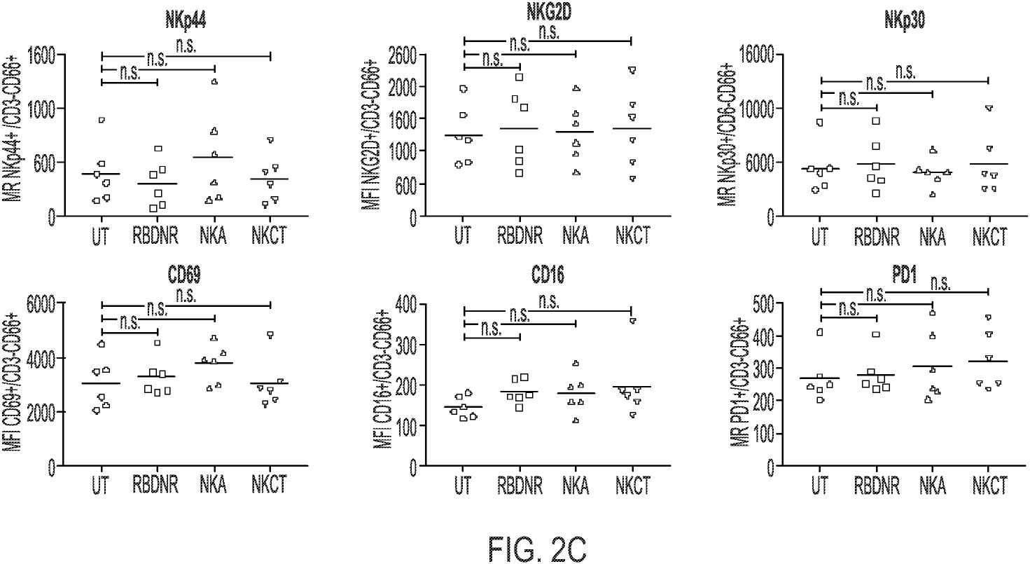

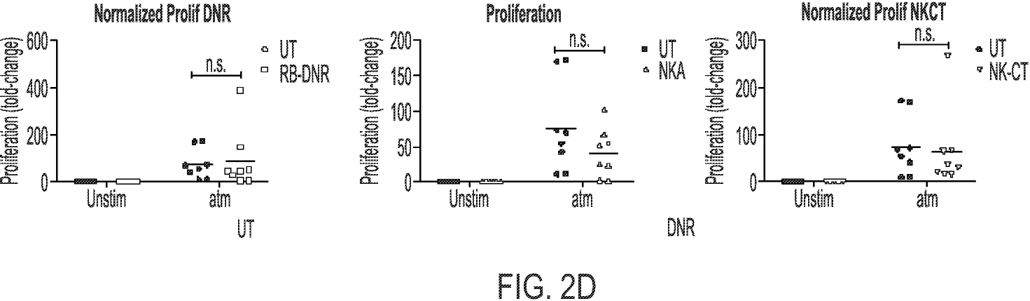

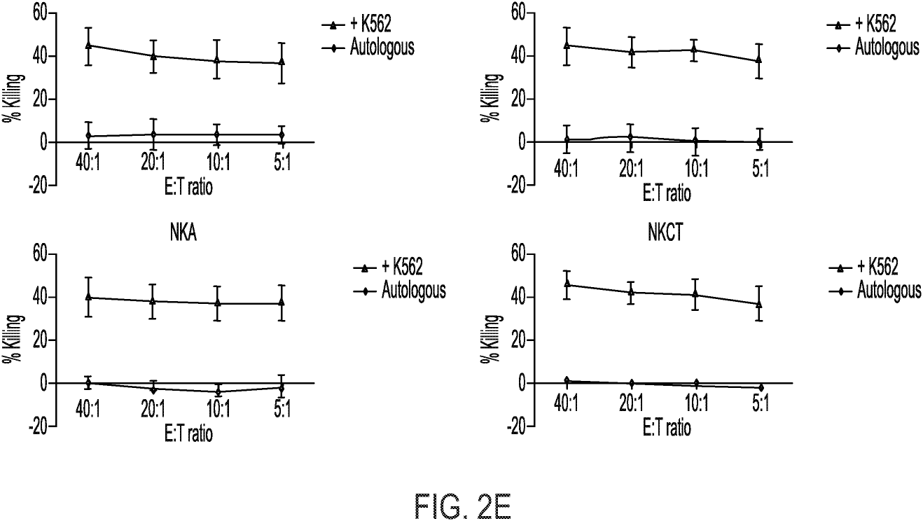

Three modified plasmids were constructed as follows (FIG. 2A): (1) RBDNR: human type II TGFβ receptor cDNA was truncated at nt597 as previously described⁶⁸ and coupled to a truncated CD19 tag and pac puromycin resistance gene via T2A sequences (2) NKA: human type II TGFβ receptor cDNA was truncated at nt597 as previously described⁶⁸ and coupled to the transmembrane and intracellular coding region of DAP12 as derived from full-length DAP 12 cDNA⁶¹, a truncated CD19 tag and a pac puromycin resistance gene via T2A sequences. (3) NKCT: human type 11 TGFβ receptor cDNA was truncated at nt597 as previously described⁶⁸ and coupled to a “SynNotch” receptor⁴⁶ composed of the Notch1 minimal regulatory region fused to the DNA binding domain for RELA (p65) and a VP64 effector domain,⁶⁹ coupled to a truncated CD19 tag and a pac puromycin resistance gene via T2A sequences. The RBDNR, NKA, and NKCT constructs were then individually integrated at the BamHI and Ncol sites of the retroviral vector SFG in order to generate plasmids of the same name. A control GFP-containing plasmid was generated elsewhere.⁷⁰ Phoenix-ecotropic cells (ATCC, Manassas, VA) were transfected with SFG:RBDNR, SFG:NKA, and SFG:NKCT, with Lipofectamine 2000 (Thermo Fisher Scientific, Waltham, MA) reagents used as per manufacturer’s protocol. Transient retroviral supernatant was collected 48 and 72 hours following transfection, and was used to transduce the PG13 stable packaging cell line (ATCC, Manassas, VA) Transduced PG13 cells were evaluated for transduction efficiency as described below, and single cell FACS sorting was performed to isolate single clonally derived producer lines. For FACS sorting, single cells that expressed high levels of CD19 and TGFβRII expression were isolated with the Becton Dickinson Influx Cell Sorter (BD Biosciences, Franklin Lakes, NJ) and selectively expanded in puromycin-containing DMEM with 10% FBS (Gibco, Thermo Fisher Scientific, Waltham, MA) and 1% Glutamax (Gibco, Thermo Fisher Scientific, Waltham, MA). Retroviral supernatants containing RBDNR, NKA, and NKCT constructs were harvested from sub-confluent PG13 cells, passed through a 0.45 µM filter, and stored at -80° C.: until needed for transduction.

### NK Cell Transduction and Expansion

Activated NK cells were harvested on day 4 of their culture, plated on retronectin-coated non-tissue culture treated plates (Takara, Japan), and transduced with RBDNR, NKA, or NKCT - containing retroviral supernatant in the presence of IL-2 (200 IU/mL). After transductions, NK cells were assessed for transduction efficiency by staining with antibodies against CD19 conjugated to allophycocyanin (BD Biosciences, Franklin Lakes, NJ) and TGFβRII conjugated to phycoerythin (R&D Systems, Minneapolis, MN). After transduction, NK cells were expanded with additional stimulations with irradiated modified K562s, as described above, and exogenous IL-2 and IL15 To enrich for phenotypic, functional, and in vivo assays, transduced NK cells were stained with CD19 microbeads (Miltenyi Biotec, Germany), and enriched by positive immunomagnetic bead selection according the manufacturer’s protocol.

### Phenotypic Assessment of NK Cells

NK cells were harvested from 21-day or 28-day cultures, washed with FACS buffer, and incubated with human FcR Blocking Reagent for 10 minutes (Miltenyi Biotec, Germany). Unmodified and modified NK cells, or cell lines, were stained with antibodies specific for NKp30, NKG2D, NKp44, CD16, PD1, CD56, CD3, DNAM1, CD19, TGFβRII (R&D Systems, Minneapolis, MN), HLA-ABC, or MICA/B. Antibodies were conjugated to FITC, PE, PerCP, APC, APC-Cy7, Pe-Cy7, or PerCP-Cy5.5 (BD Biosciences, Franklin Lakes, NJ unless otherwise identified). Samples were ran on the Accuri C6 (BD Biosciences, Franklin Lakes, NJ) or CytoFLEX S (Beckman Coulter, Indianapolis, IN) flow cytometers and analysis conducted using Flow Jo 7.6.5 (FlowJo LLC, Ashland, OR). For staining of intracellular or nuclear proteins, cells were fixed with 16% formaldehyde, and permeabilized with ice-cold 100% methanol, prior to staining with antibodies to phospho-Smad2/3, and RELA (p65) from BD Biosciences. Voltages were set based on unstained cells, compensation calculated using single-stained controls, positive staining was definted with fluorescence minus one (FMO) controls, and mean fluorescence intensity calculated. Culture supernatant was harvested from 21-day or 28-day NK cultures and stored at -80° C. until needed. To assess the cytokine profile of transduced and untransduced NK cells, supernatant was thawed and used in the Bio-Plex Human Cytokine 17-plex Assay according to the manufacturer’s instructions (Bio-Rad Laboratories, Hercules, CA). For phenotypic assessment of unmodified and modified NK cells after exposure to TGFβ. NK cells were cultured with 10 ng/ml. TGFβ (activated with 4 mM HCl) added every other day. After 5 days, NK cells and supernatant were isolated and examined by flow cytometry or multiplex assays as described above. For examination of cellular proliferation, NK cells were labeled with carboxyfluorescein succinimidyl ester (CFSE) as per manufacturer’s protocol (Thermo Fisher Scientific, Waltham, MA) and co-cultured with modified K562 cells for 2-4 days.

### Functional Assessment of NK Cells

To determine the cytolytic killing abilities of unmodified and modified NK cells in various conditions, standard ⁵¹Cr release cytotoxicity assays were performed. NK cells were incubated with ⁵¹Cr-labeled target cells (unmodified K562s, SHSY5Y cell lines - loaded with 10 µCi ⁵¹Cr per 10000 cells) at 40:1, 20:1, 10:1, and 5:1 ratios for 5 hours in triplicate in roundbottomed 96-well plates. Target cells were incubated in media alone or in 5% Triton X-100 (Sigma, St. Louis MO) to determine spontaneous and maximum release, respectively, and ⁵¹Cr release counts were obtained with a MicroBeta2 gamma-counter (Perkin Elmer, Waltham, MA). The percent killing was determined by the following formula: (experimental count - spontaneous count) / (maximum count - spontaneous count) x 100%. For functional assessment of unmodified and modified NK cells after exposure to TGFβ, NK cells were cultured with 10 ng/ml. TGFβ (activated with 4 mM HCl) added every other day. After 5 days, NK cells were isolated and used in cytotoxicity assays as described above.

### Molecular Assessment of NK Cells After TGFβ Exposure

To examine the molecular effects of TGFβ, unmodified and modified NK cells were cultured with 10 ng/ml, TGFβ (activated with 4 mM HCl) at 37° C. At 30 mins, 1 hr, 3 hr, 24 hr, 48 hr, and 72 hr post-TGFβ addition protein was isolated for molecular assessment. Briefly, unmodified or modified NK cells were pelleted and resuspended in RIP A lysis buffer (Thermo Fisher Scientific, Waltham, MA) containing protease inhibitor and phosphatase inhibitor cocktails (Roche Diagnostics, Indianapolis, IN) Following 10 minutes of incubation at 4° C., protein was isolated and particulate matter removed by filtration with Ultafree-CL centrifugal filter units (EMD Millipore, Burlington, MA). Protein was quantified with a Pierce BCA Protein Assay Kit (Thermo Fisher Scientific, Waltham, MA). For Western blots, 25 ug of protein (per gel) was mixed 1:1 with Laemmli buffer (Bio-Rad Laboratories, Hercules, CA), heated at 98° C. for 10 mins, sonicated, and loaded into wells of a precast Bolt 4-12% Bis Tris Plus gel (Thermo Fisher Scientific, Waltham, MA) The iBlot 2 Dry Blotting System (Thermo Fisher Scientific, Waltham, MA) was used to transfer gels on to PVDF membranes, which were probed with rabbit anti-human antibodies against vinculin (Abeam, Cambridge, MA), Smad2 (Cell Signaling Technology, Danvers, MA), or phospho-Smad2 (Ser465/467, Cell Signaling Technology, Danvers, MA). Following overnight incubation, membranes were probes with an anti-rabbit Europium conjugated secondary (Molecular Devices, San Jose, CA), and protein expression quantitated with the Scan Later Western blot system (Molecular Devices, San Jose, CA) Western blots were analyzed and quantified using ImageJ software. For protein multiplexing, 30 ug of protein lysate was isolated and used in the TGFβ Signaling Pathway Magnetic Bead 6-plex Cell Signaling Multiplex Assay (EMD Millipore, Burlington, MA) as per manufacturer’s instructions and protein expression of phospho-Akt (Ser473), phospho-ERK (Thr185/Tyr187), phospho-Smad2 (Ser465/467), phospho-Smad3 (Ser423/425) quantitated with Luminex xMap detection, based on positive and negative quantified protein controls.

### Mice and in Vivo Experiments

Male and Female NSG (NOD.Cg-Prkdc^{scid}Il2rg^{lm1Wjl}/SzJ) mice were purchased from Jackson Laboratories (Bar Harbor, ME) and bred in-house in accordance with approved protocols with the Institutional Animal Care and Use Committee at Children’s National Health System. For in vivo neuroblastoma treatment experiments, 6-10 week old male and female mice were preconditioned with sublethal irradiation (300 cGy) and inoculated with 2.5e⁶ SHSY5Y-luc cells, administered subcutaneously in the dorsal flank of animals. Animals were treated immediately following inoculation, a model commonly used in the field⁵⁰, with systemic administration of 15e⁶ unmodified or modified NK cells via tail veins. For long-term studies, animals received weekly doses of 5-10e⁶ unmodified or modified NK cells, administered systemically (5 doses in total). All mice were treated with 0.2 ug human IL-2, administered intraperitoneally every other day over the course of their cell therapy doses. For examination of tumor progression, animals were imaged every other day with the IVIS Lumina 100 (Perkin Elmer, Waltham, MA), and images were scaled to the same minimum and maximum photon distribution prior to analysis. Animals were injected with 150 mg/kg Xeno-Light D-Luciferin (Perkin Elmer, Waltham, MA) 10 minutes prior to imaging with the IVIS, during which time animals were anesthetized with 2% isoflurane. Bioluminescent images were captured with 15 s exposure, with small binning and f-stop 2, and total bioluminescence was quantified by photon counts under individual murine regions of interest. For analysis of NK cell persistence, blood was collected at designated time points from submandibular veins with Goldenrod Animal Lancets (Braintree Scientific Inc. Braintree, MA) and stored in K2EDTA-containing Microtainer tubes (BD Biosciences, Franklin Lakes, NJ) at -80° C.

### Assessment of NK-Cell Persistence in Vivo

Transduced NK cells were detected and quantified in the peripheral blood using digital droplet PCR (ddPCR) methods. Collected in vivo blood samples were thawed and RNA was extracted using the Whole Blood Quick-RNA kit according to the manufacturer’s instructions (Zymo Research, Irvine, CA). cDNA was prepared from 2000 ng of isolated RNA by performing PCR amplification with RT buffer, dNTP Mix, MultiScribe RT, RNAse inhibitor, random primers, and nuclease free water according to the High Capacity RT cDNA kit (Thermo Fisher Scientific, Waltham, MA). 10 uL cDNA was then combined with ddPCR Supermix (Bio-Rad Laboratories Inc, Hercules, CA) containing a final concentration of 900 nM forward primer, 900 nM reverse primer, and 250 nM probe, and samples were plated on a 96-well microamp plate and loaded onto the AutoDG Automated Droplet Generator (Bio-Rad Laboratories Inc, Hercules, CA) set to produce 20000 droplets/sample. After droplet generation, the plate was placed in a thermal cycler for amplification at the following conditions: 95° C. for 10 minutes, 40 cycles of [94° C. for 30 seconds, 60° C. for 1 minute], followed by 98° C. for 10 minutes and holding at 12° C. Finally, the plate was loaded in the QX200 Droplet Reader (Bio-Rad Laboratories Inc, Hercules, CA) for droplet quantification and analyses. For identification of RBDNR-transduced NK cells we used a human primer/probe combination that spanned a 129 bp region in the truncated TGFβ domain: forward primer (5′-GACATGATCGTGACCGATAACA-3′), reverse primer (5′-GCAGATAGAGGTGATGGAACAA-3′), and probe (5′-FAM-AGTTCTGTGACGTGCGGTTTAGCA-TAMSp-3′). For identification of NKA and NKCT-transduced NK cells we used a human primer/probe combination that spanned a 126bp region in the truncated TGFβ domain: forward primer (5′-AACAATGGCGCCGTGAAGTTCC-3′), reverse primer (5′-CCTCCTGTGGCTTCTCGCAGAT-3′), and probe (5′~FAM~ AGTTCTGTGACGTGCGGTTTAGCA-TAMSp-3′). For identification of mock (GFP)-transduced NK cells we used a human primer/probe combination as follows: forward primer (5′-CCGCCGACACCAGACTAAG-3′), reverse primer (5′GCTGAACTTGTGGCCGTTTAC-3′), and probe (5′-FAM-TGCCATGGTGAGCAAGGGCG-TAMSp-3′). All samples were multiplexed; in addition to the above FAM-TAMSp probes and corresponding primers, all samples were assessed for murine TBP gene content using commercial TBP forward and reverse primers with a VIC-MGB probe (Thermo Fisher Scientific, Waltham, MA). Concentrations of each sample were extrapolated using the standard curve generated from TBP quantification and absorbance readings from 1:4 serially diluted samples. All samples were analyzed in duplicate, cDNA from in vitro transduced NK cells were used as positive controls, and individual sample results were normalized to murine TBP gene content in each sample.

### Statistical Analysis and Schematics

All experiments were performed in duplicate or triplicate, with sample sizes indicated in each corresponding figure legend. Data was analyzed using GraphPad Prism software (GraphPad, La Jolla, CA). Comparisons between untransduced, RBDNR, NKA, and NKCT data were performed using Student’s t-test or Chi-squared tests, with p<0.05 as considered significant. For in vivo experiments, we performed the log-rank (Mantel-Cox) test for Kaplan-Meier generated survival data, with p<0.05 as considered significant. Schematic signaling diagrams were generated using Biorender (Toronto, Canada).

### Example 2. Unmodified and Variant TGFβ Receptor-Modified NK Cells Are Phenotypically and Functionally Similar

Cord blood-derived NK cells^{24,28,29} were isolated and stimulated with irradiated feeder cells and supplemental human IL-2 and IL-15.^{21,48} Four days after stimulation, NK cells were divided in to four groups: untransduced (UT), RBDNR-transduced, NKA-transduced, and NKCT-transduced (FIG. 2A), as described. Cord-blood derived NK cells were successfully transduced with RBDNR, NKA, or NKCT variant TGFβ receptors, as indicated by surface staining of TGFβRII and CD19 (FIG. 2B, TGFβRII+CD19+: UT 1.92±2.64% vs. RBDNR 43.9±24.1% vs. NKA 43.2±27.1% vs. NKCT 39.1±26.3%, CD19+: UT 1.86-±3.57% vs. RBDNR 42.6±27.6% vs NKA 43.9±30.2% vs NKCT 36.9±29.4%, n>30). Staining for natural cytotoxicity receptors NKp44 and NKp30 indicated no significant difference in expression on transduced NK cells as compared to their untransduced counterparts (NKp44: UT 27.4±15.6% vs. RBDNR 25.1±18.0% vs. NKA 31.9±14.9% vs. NKCT 26.4±18.2% p>0.05, NKp30: UT 41.1±27.7% vs. RBDNR 44.2±28.9% vs. NKA 41.7±26.5% vs. NKCT 41.9±31.4% p>0.05, n>5, FIG. 2C). Similarly no impairment in the expression of other NK cell surface markers NKG2D, CD69, CD16, or PD1 was found (p>0.05, n>5, FIG. 2C). NK cells were labeled with CFSE and co-cultured with unlabeled modified K562s. Analysis of CFSE dilution over three days by flow cytometry demonstrated no changes in NK cell proliferation following transduction with RBDNR, NKA, or NKCT receptors (fold-change compared to unstimulated; UT 75.3-fold vs. RBDNR 88.5-fold vs. NKA 41.3-fold vs. NKCT 64.2-fold, p>0.05, n>5, FIG. 2D). ⁵¹Cr-based cytotoxicity assays with untransduced and transduced NK cells demonstrated maintenance of cytolytic killing ability against K562 target cells occurring in each condition (UT vs. RBDNR vs. NKA vs. NKCT p>0.05, n>5, FIG. 2E). These in vitro characterizations demonstrated that introducing an engineered TGFβ receptor did not affect NK cell phenotype or function.

### Example 3. TGFβ Receptor-Modified NK Cells Exhibit Protection From Downstream Molecular Effects of Exogenous TGFβ

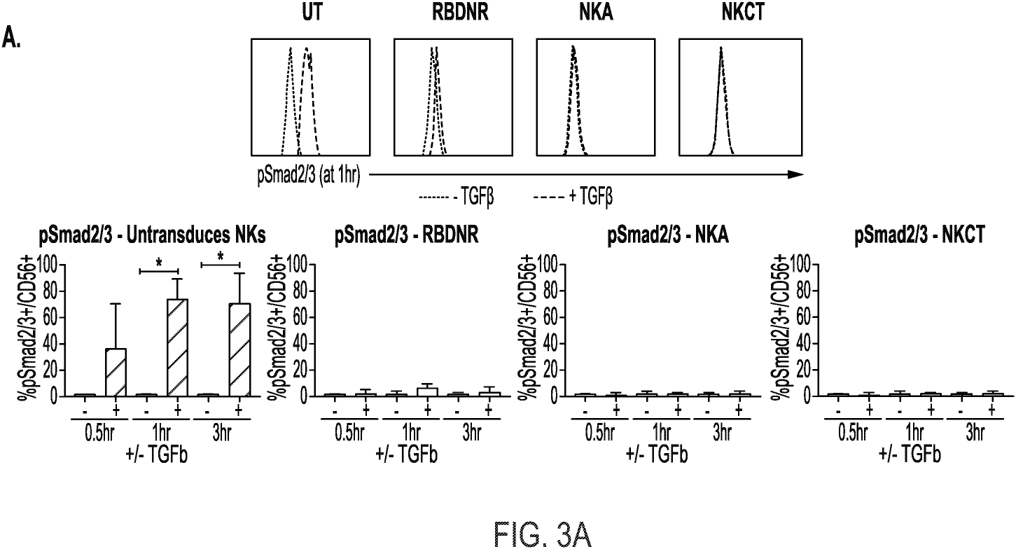

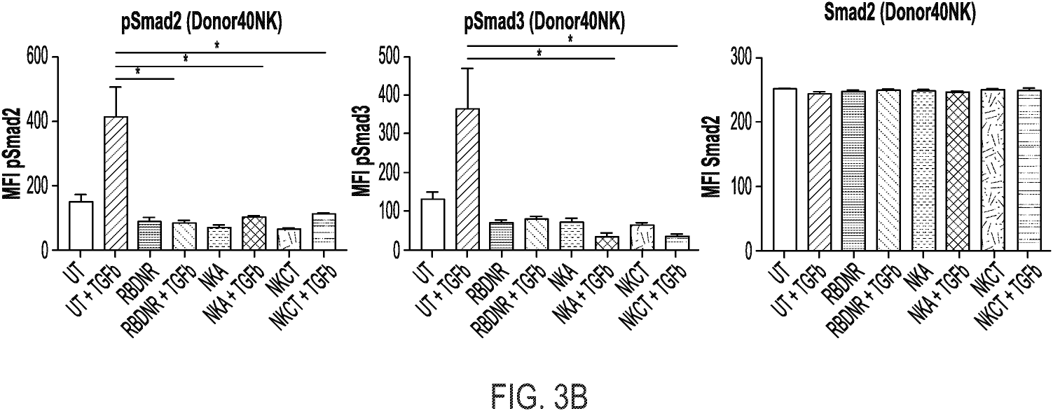

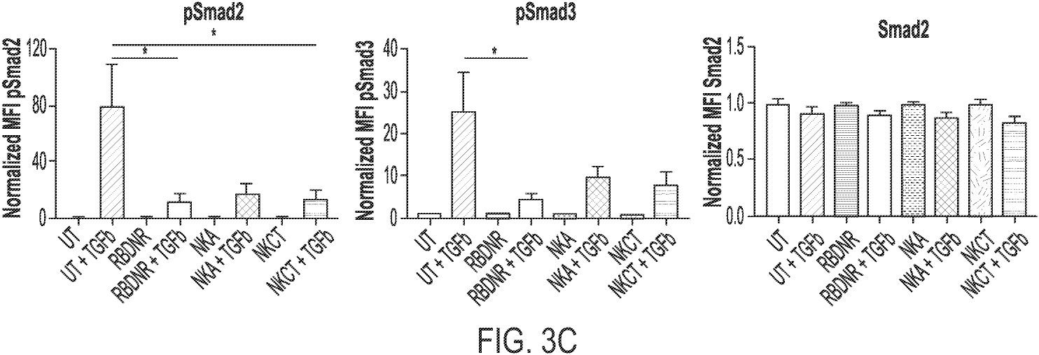

Exposure to TGFβ initiates a cascade originating with the phosphorylation of intracellular Smad2 and Smad3 proteins.³¹ To investigate the protective ability of RBDNR, NKA, and NKCT constructs at preventing TGFβ-mediated signaling, we co-cultured untransduced, RBDNR, NKA, and NKCT-transduced NK cells with TGFβ. Cells were harvested 0.5, 1, or 3 hours after TGFβ exposure and either lysed to isolate protein or assayed by flow cytometry Flow cytometry demonstrated rapid phosphorylation (Ser465/467) of Smad2/3 occurring when untransduced NK cells were exposed to TGFβ (pSmad2/3: UT 1.36±0.95% vs. UT+TGFβ UT 73.9±20.5%,p=0.04 at 1 hr, n>3, FIG. 3A), which did not occur in NK cells transduced with either RBDNR, NKA, or NKCT receptors following TGFβ exposure (p>0.05 at 1 hr, p>0.04 at 3 hr, n>3, FIG. 3A) Similarly, evaluation of Smad2 (Ser465/467) and Smad3 (Ser423/425) phosphorylation from protein lysate isolated from untransduced and transduced cells after 1 hr of TGFβ exposure further demonstrated the protective effect that of the TGFβ receptor-modifications conferred to NK cells. Protein lysate results are demonstrated from one representative NK line (pSmad2 UT+TGFβ vs. RBDNR+TGFβ p=0.034, UT+TGFβ vs. NKA+TGFβ p=0.038, UT+TGFβ vs. NKCT+TGFβ p=0.04; pSmad3 UT+TGFβ vs. NKA+TGFβ p=0.045, UT+TGFβ vs. NKCT+TGFβ p=0.045, n>5; FIG. 3B) as well as from pooled NK donor lines (pSmad2 UT+TGFβ vs. RBDNR+TGFβ p=0.025, UT+TGFβ vs. NKCT+TGFβ p=0.031; pSmad3 UT+TGFβ vs. RBDNR+TGFβ p=0.037, n>5; FIG. 3C). These results demonstrated phosphorylation of Smad2 occurring in only UT NK cells exposed to TGFβ, while production of Smad2 protein remained constant throughout all samples

### Example 4. TGFβ Receptor-Modified NK Cells Have Increased Expression of Activation Markers and Maintain Functionality in the Presence of TGFβ

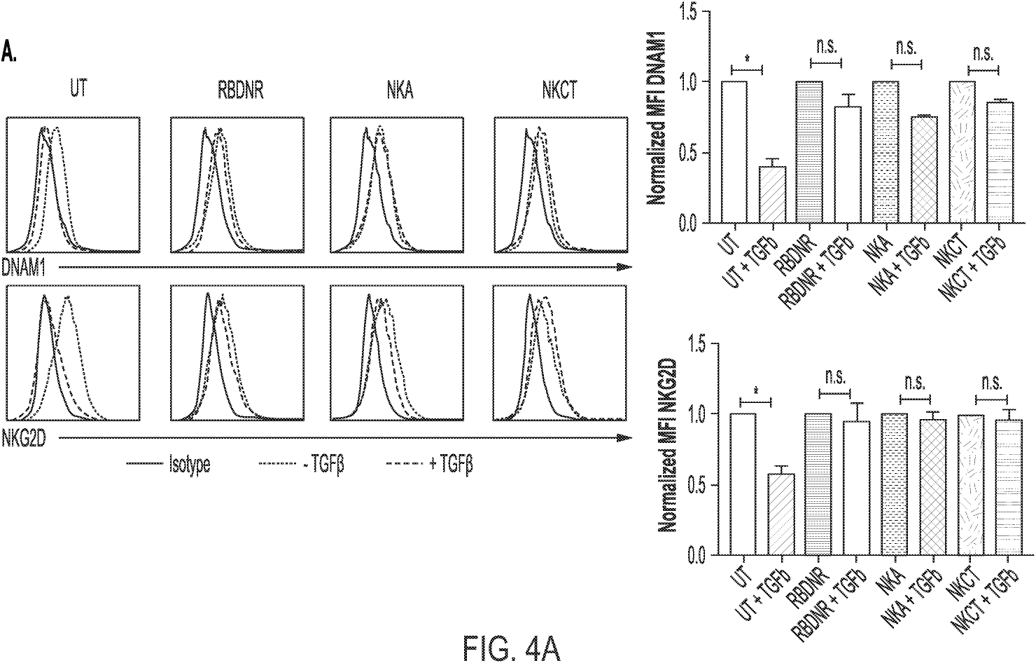

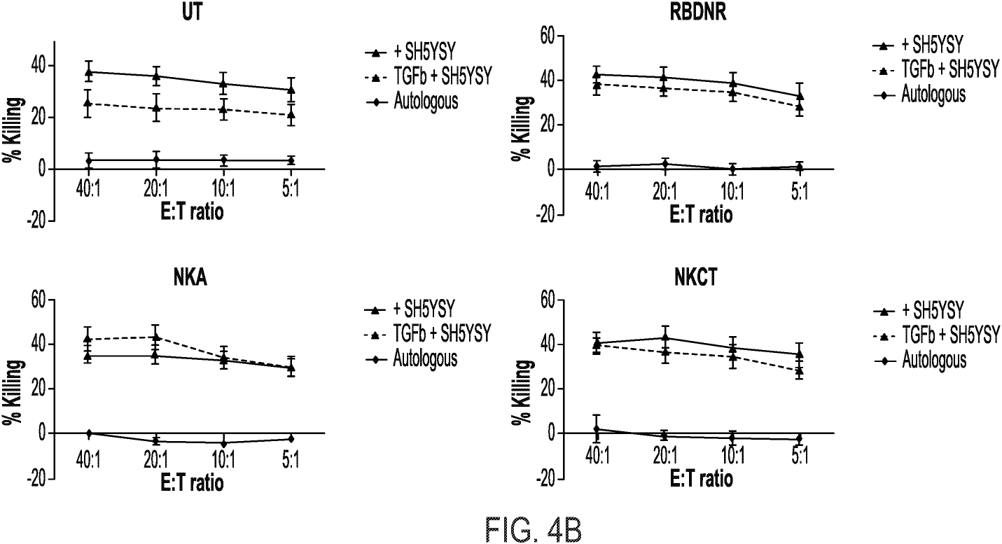

To assess whether the protection from the molecular changes occurring after TGFβ exposure translated to a phenotypic or functional advantage, untransduced and RBDNR, NKA, and NKCT-transduced NK cells were examined after 5-days of TGFβ co-culture. Flow cytometry revealed decreases in the expression of DNAX Accessory Molecule-1 (DNAM1 fold-change from non-TGFβ exposed: UT 0.39-fold, p=0.0163, n>5, FIG. 4A) and in NKG2D (fold-change from non-TGFβ exposed: UT 0.58-fold, p=0.04, n>5, FIG. 4A) in untransduced NK cells following exposure to TGFβ. This downregulation in surface markers was not observed in RBDNR, NKA, or NKCT-transduced NK cells, which all exhibited protection from these TGFβ-mediated phenotype impairments (p>0.05, n>5, FIG. 4A). Likewise, whereas untransduced NK cells exhibited dose-dependent cytotoxicity against SHSY5Y neuroblastoma cells (38.2±4.69% killing at E:T ratio 40:1), they demonstrated impaired cytolytic activity (24.6±4.58% killing at E:T ratio 40:1) following pre-culture with TGFβ. This impairment in cytolytic ability was not demonstrated when NK cells transduced to express the variant TGFβ-receptors (RBDNR, NKA, or NKCT) were assessed following pre-treatment with TGFβ (FIG. 4B), suggesting their functional superiority at killing target cells amidst a TGFβ-rich environment.

### Example 5. DAP12 and RELA-Containing TGFβ Receptor Variant NK Cells Demonstrated Increased Expression of Molecular Activation Markers Following Exposure to TGFβ

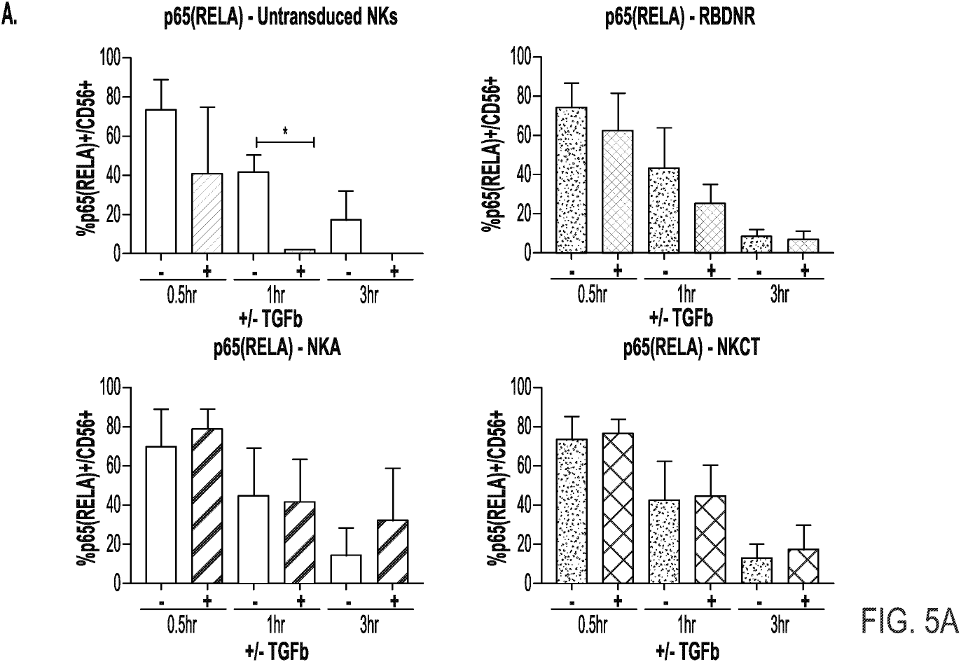

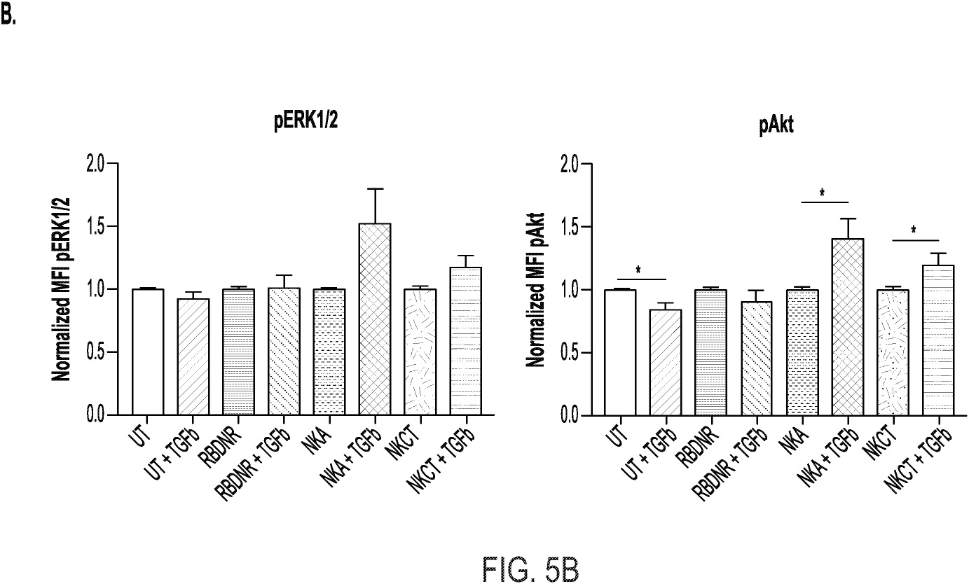

To examine the induction of NK cell activation, we co-cultured untransduced, RBDNR, NKA, and NKCT-transduced NK cells with TGFβ. Cells were harvested 0.5, 1, or 3 hours after TGFβ exposure and either lysed to isolate protein or assayed by flow cytometry Flow cytometry demonstrated decreasing levels of RELA (p65) occurring in untransduced NK cells at one and three-hours post-TGFβ exposure (UT 42.3±13.7% vs. UT+TGFβ UT2.02±1.08%, p=0.02 at 1 hr, UT 21.5±11.5%vs. UT+TGFβ UT 0.47±0.46%, p=0.18 at 3 hr, n>3, FIG. 5A). Similar trends in RELA were seen in RBDNR-transduced NK cells at one-hour post-TGFβ exposure (p=0.31 at 1 hr, p=0.18 at 3 hr, n>3, FIG. 5A). NK cells transduced with either NKA or NKCT variant TGFβ receptors demonstrated unaltered p65 expression following exposure to TGFβ (NKA p=0.92 at 1 hr, p=0.61 and 3 hr, n>3; NKCT p=0.96 at 1 hr, p=0.75 at 3 hr, n>3), suggesting that NFκB-mediated signaling was occurring in these cells. Evaluation of ERK1/2 (Thr185/Tyr187) and Akt (Ser473) phosphorylation occurring in protein lystate isolated from untransduced and transduced cells after 1 hr of TGFβ exposure further demonstrated the activation occurring in NKA and NKCT-transduced NK cells. While untransduced or RBDNR-transduced NK cells exhibited decreased or unchanged levels of Akt phosphorylation (UT vs. UT+TGFβ p=0.0075, RBDNR vs. RBDNR+TGFβ p=0.282, n>5; FIG. 5B), NK cells equipped with the activation-inducing TGFβ variants exhibited increased Akt phosphorylation (NKA vs. NKA+TGFβ p=0.0.013, NKCT vs. NKCT+TGFβ p=0.0.037, n>5; FIG. 5B). Taken together, these results suggest that NK cells transduced to express the NKA or NKCT TGFβ receptor variants demonstrated heightened NK activation, consistent with the observed molecular changes occurring along the NFκB and PI3K signaling pathways.

### Example 6. Treatment With a Single-Dose of TGFβ Receptor-Modified NK Cells Slows Neuroblastoma Tumor Progression in Vivo

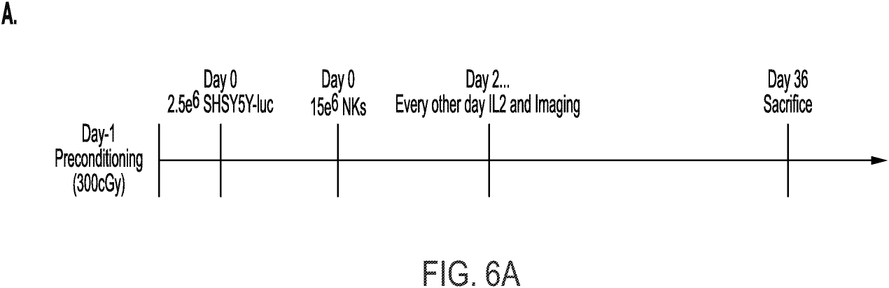

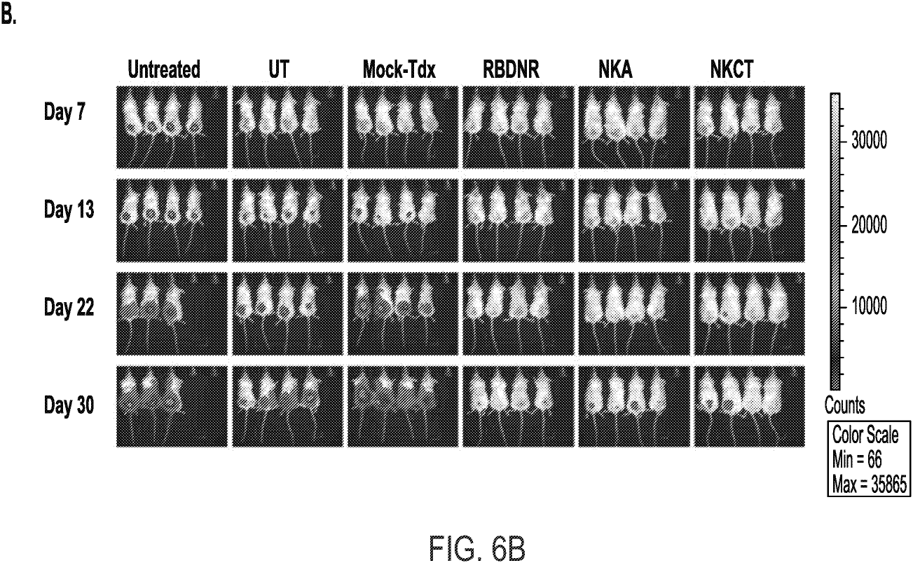

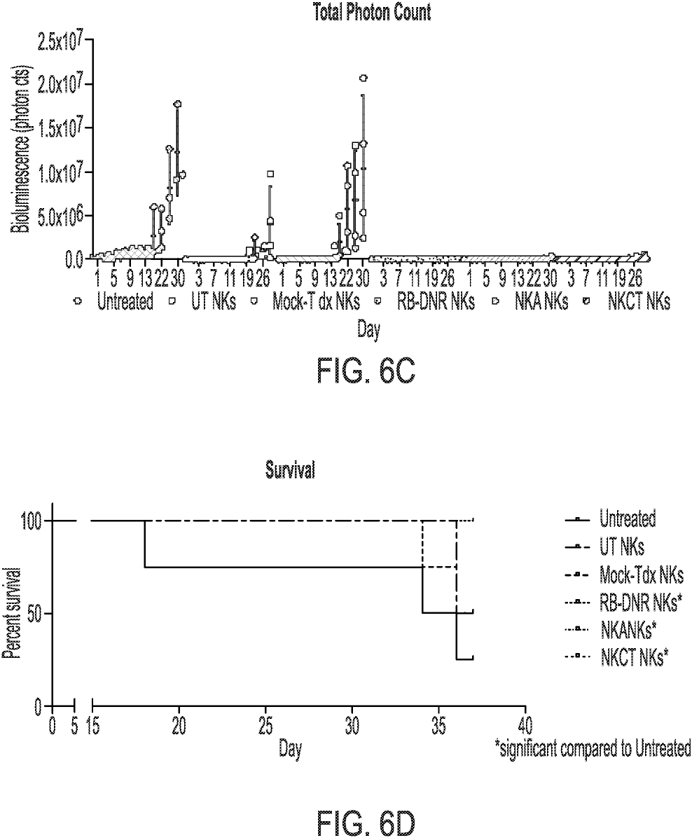

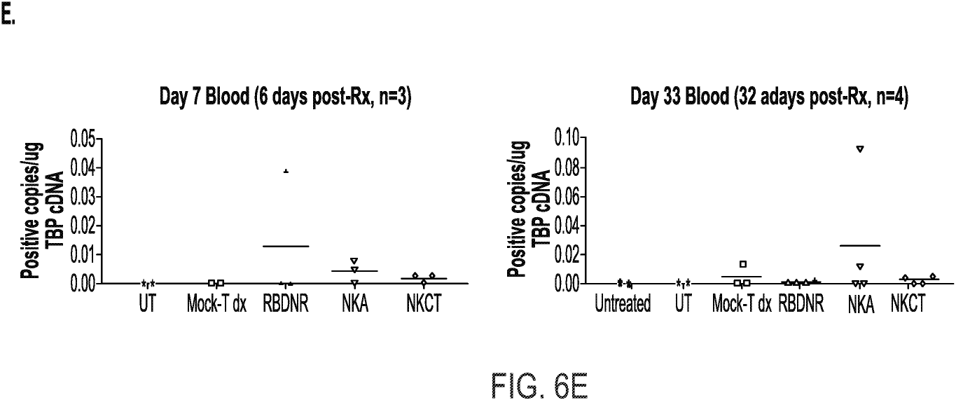

A xenograft model of human neuroblastoma using SHSY5Y human neuroblastoma cells was established,⁴⁹ inoculated subcutaneously in pre-conditioned immunodeficient animals Animals were randomly assigned to six treatment groups: untreated, untransduced NK cells (UT), mock GFP-transduced NK cells (Mock-Tdx), RBDNR-transduced NK cells (RBDNR), NKA-transduced NK cells (NKA), and NKCT-transduced NK cells (NKCT) Following inoculation, animals were immediately⁵⁰ treated systemically with 15e⁶ NK cells, and were monitored as well as administered intraperitoneal IL-2 every other day for the duration of the study (FIG. 6A). Tumor growth was monitored over time by quantifying bioluminescence (total photon counts) of animals imaged with the IVIS system every other day, analyzed with a normalized photon scale^{51,52}. Bioluminescence data revealed that tumor burden rapidly increased after 10-14 days for animals left untreated or treated with untransduced or mock-transduced NK cells (FIGS. 6B, 6C). In contrast, treatment with RBDNR, NKA, or NKCT-transduced NK cells lead to improved control of tumor progression and conferred a survival advantage compared to untreated animals (untreated vs. RBDNR p=0.040, untreated vs. NKA p=0.04, untreated vs. NKCT p=0.04; n=4 mice/group, FIG. 6D). Six and thirty-two days following cell treatment, peripheral blood was obtained from select animals to quantify the presence of genetic content from mock transduced, RBDNR, NKA, or NKCT NK cells as measured with ddPCR. At day 7, transduced NK cells were detectable in the blood of animals treated with each of the three TGFβ-receptor variant NK cells (RBDNR ⅓ animals tested, NKA ⅔ animals tested, NKCT ⅔ animals tested. FIG. 6E). At day 33, transduced NK cells were detectable in the blood of animals treated with mock and each of the three TGFβ-receptor variant NK cells (Mock-Tdx ⅓ animals tested, RBDNR ¼ animals tested, NKA 2/4 animals tested, NKCT 2/4 animals tested, FIG. 6E). Higher levels of positive copies/ug of NKA-transduced cells may suggest an in vivo effect of the DAP12 motif on NK cell persistence in a TGFβ-rich environment. Although treatment with TGFβ receptor-modified NK cells translated to better performance against neuroblastoma in vivo, treated animals at the end of study presented with evidence of persistent tumor relapse, which was not palpable but we found to be biologically active by bioluminescence. As such, we hypothesized that repeat dosing, as in common clinical practice, would be required for establishment of long-term anti-tumor effects.

### Example 7. Repeat Dosing With TGFβ Receptor-Modified NK Cells Achieves Enhanced Survival and Tumor Eradication in a Xenograft Model of TGFβ-Secreting Neuroblastoma

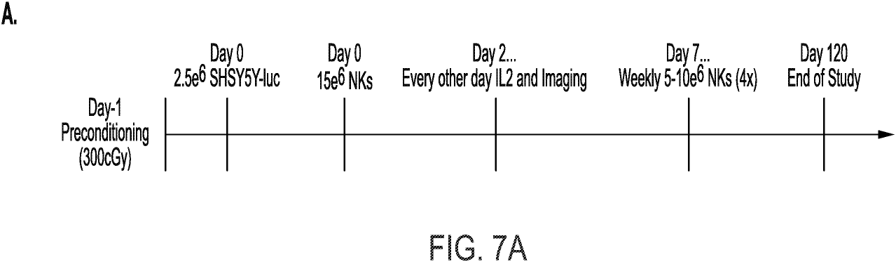

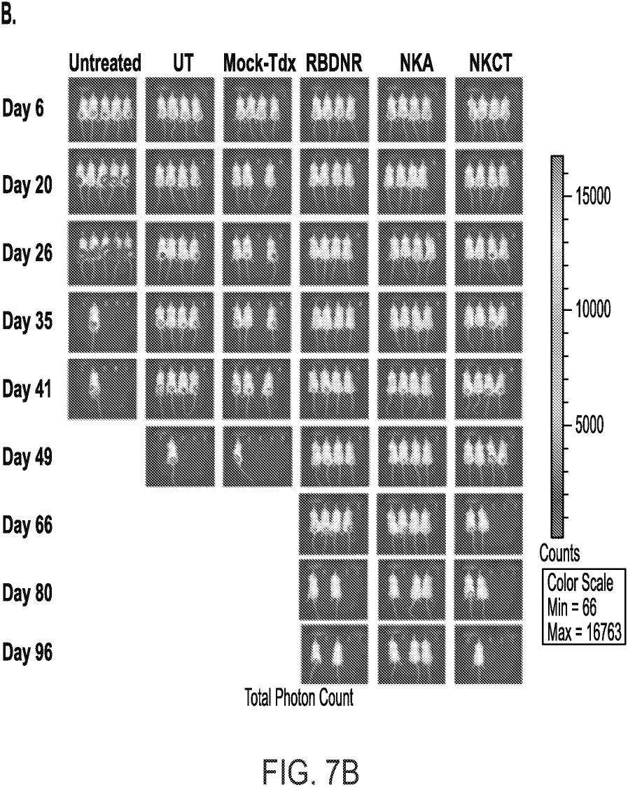

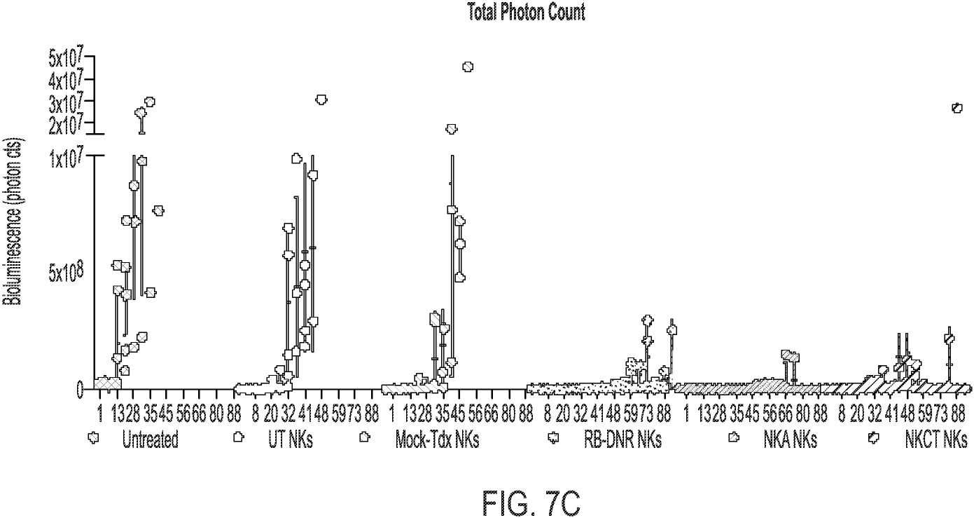

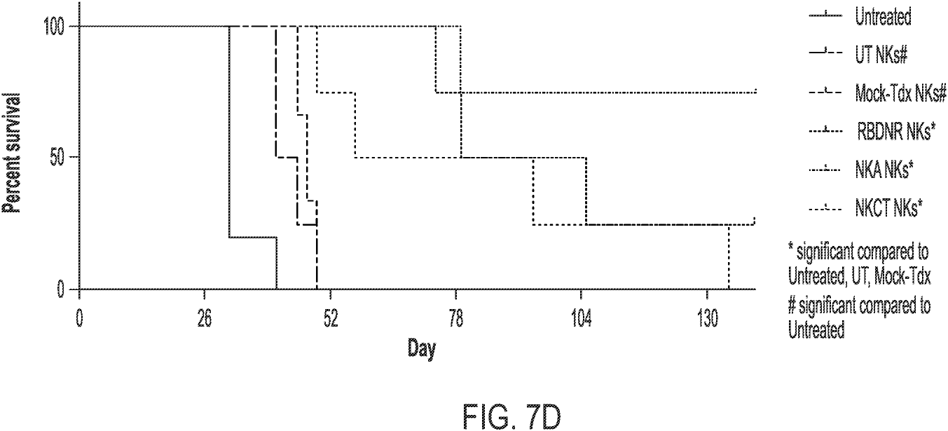

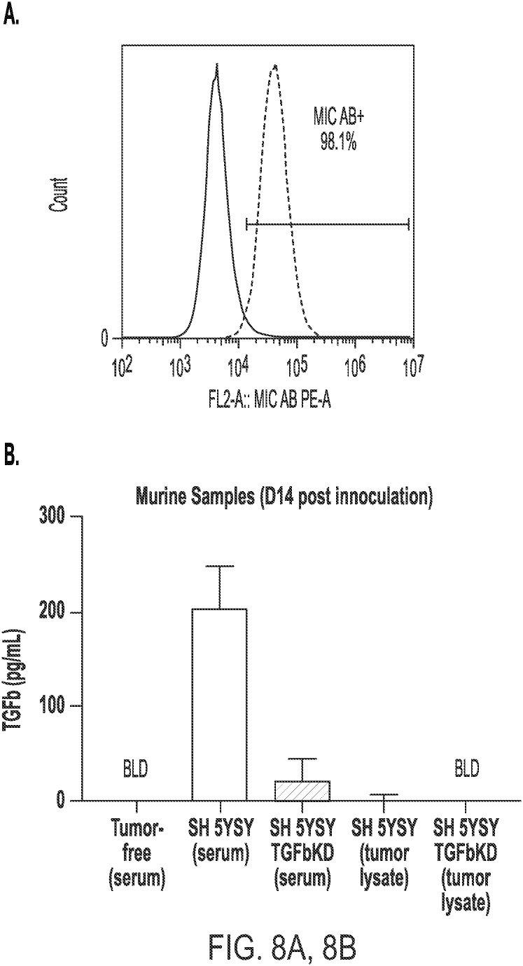

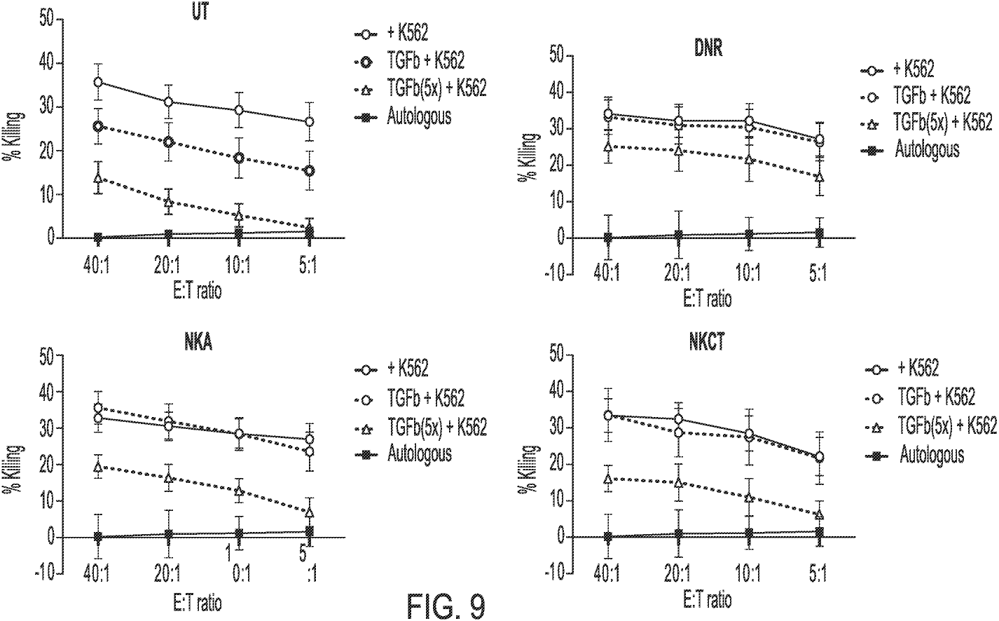

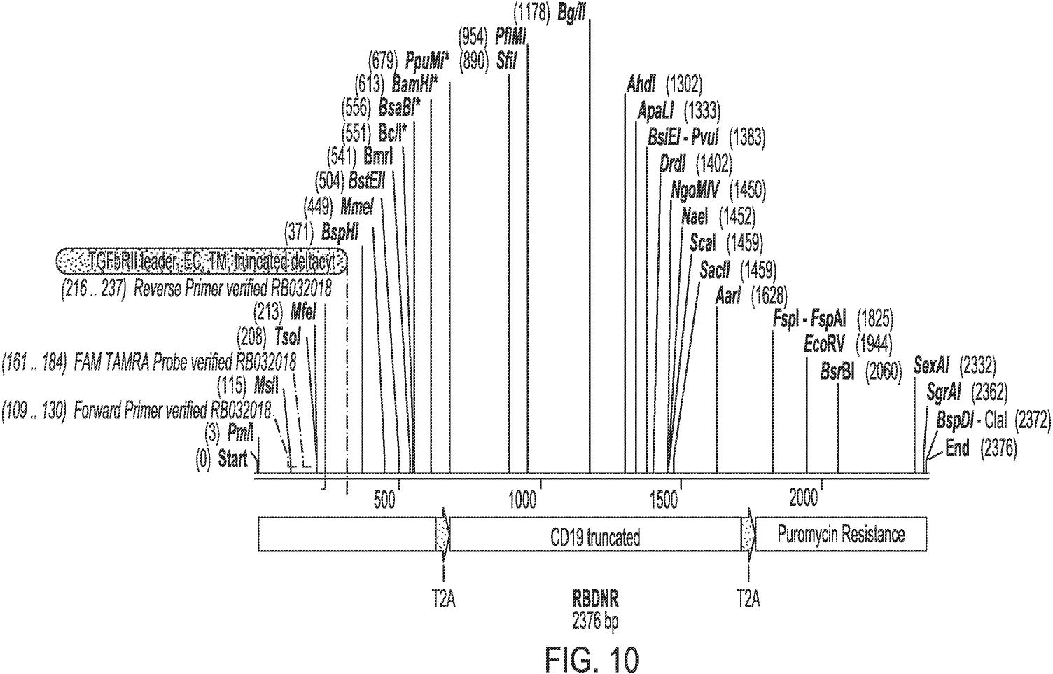

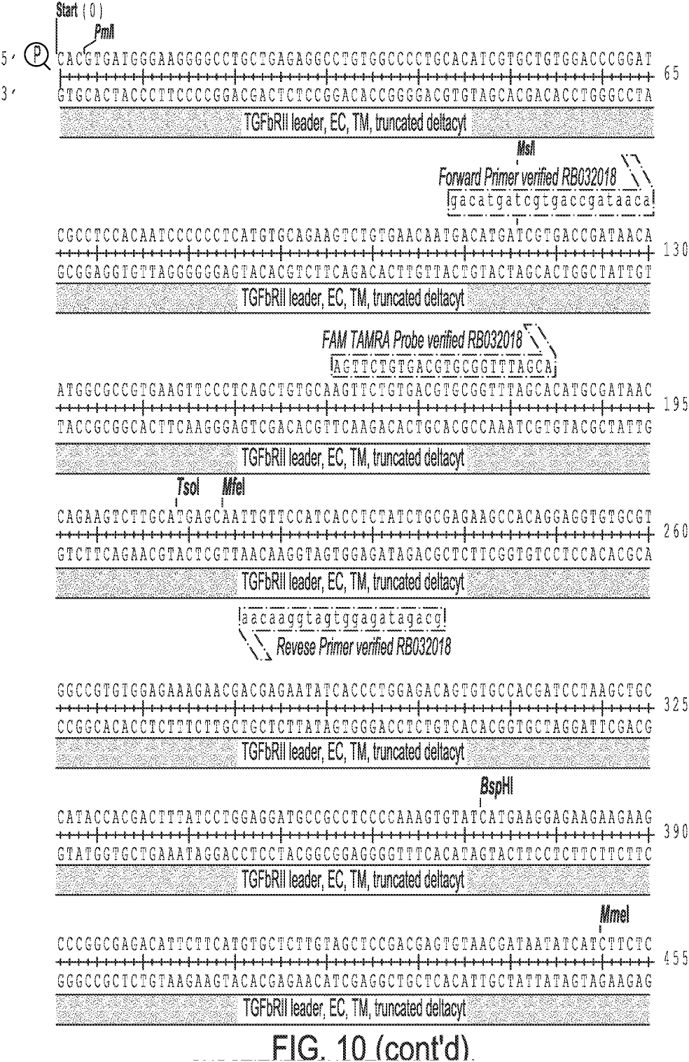

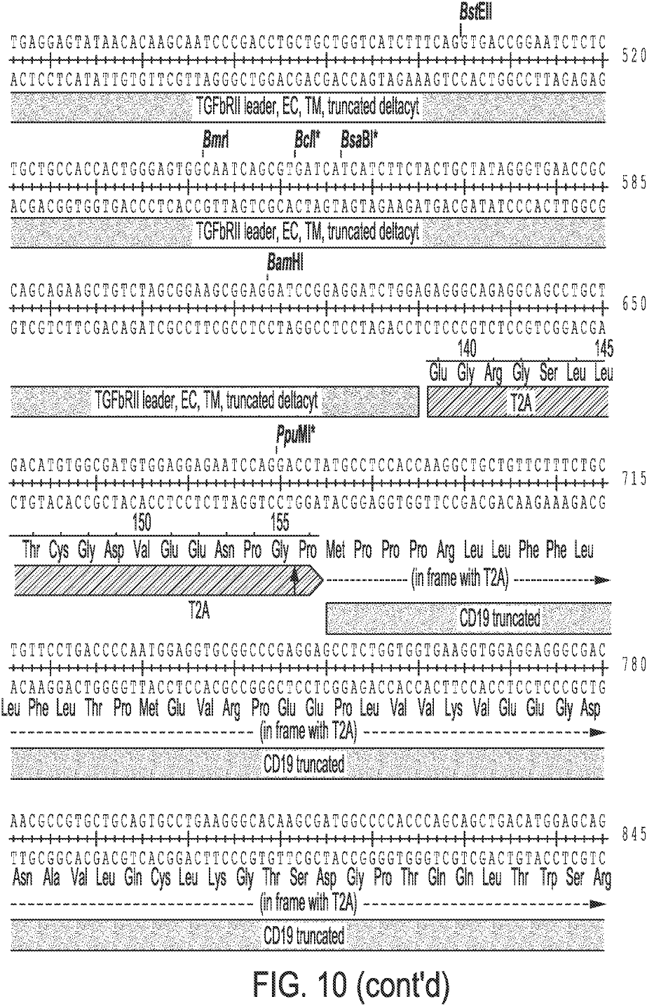

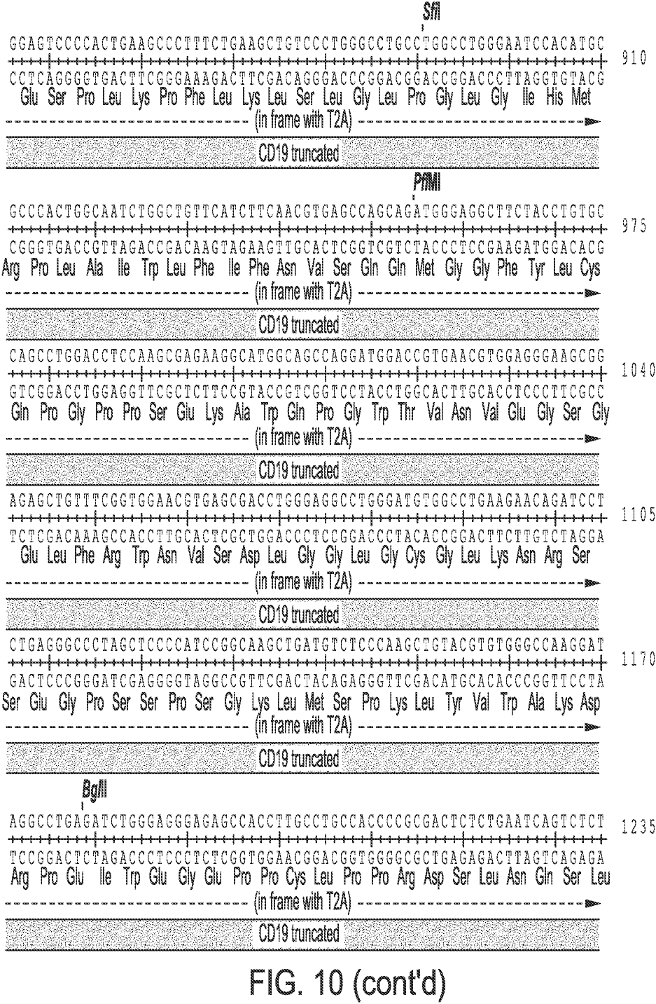

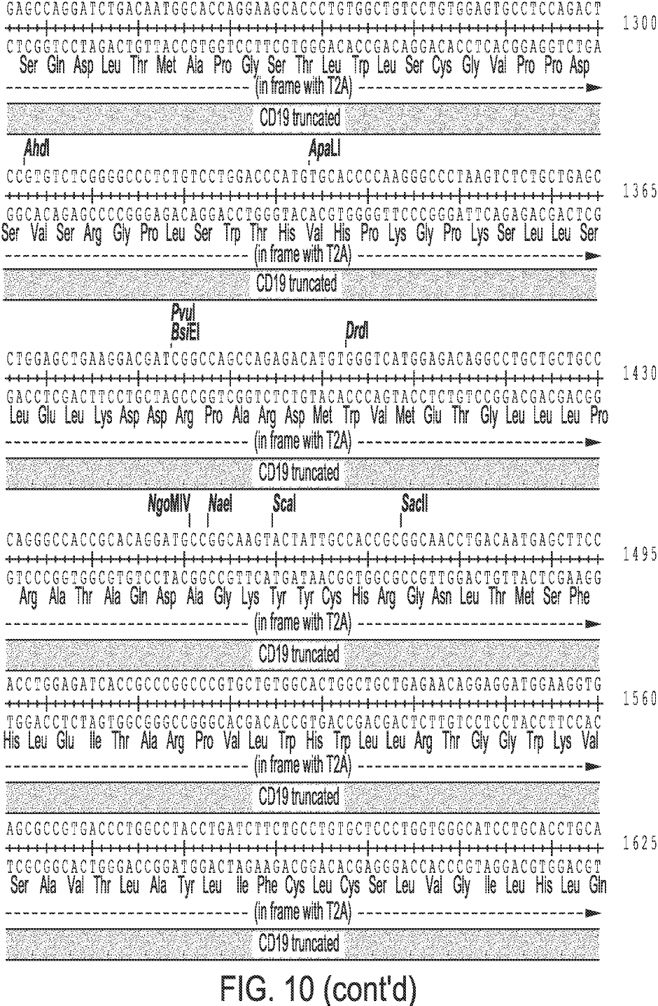

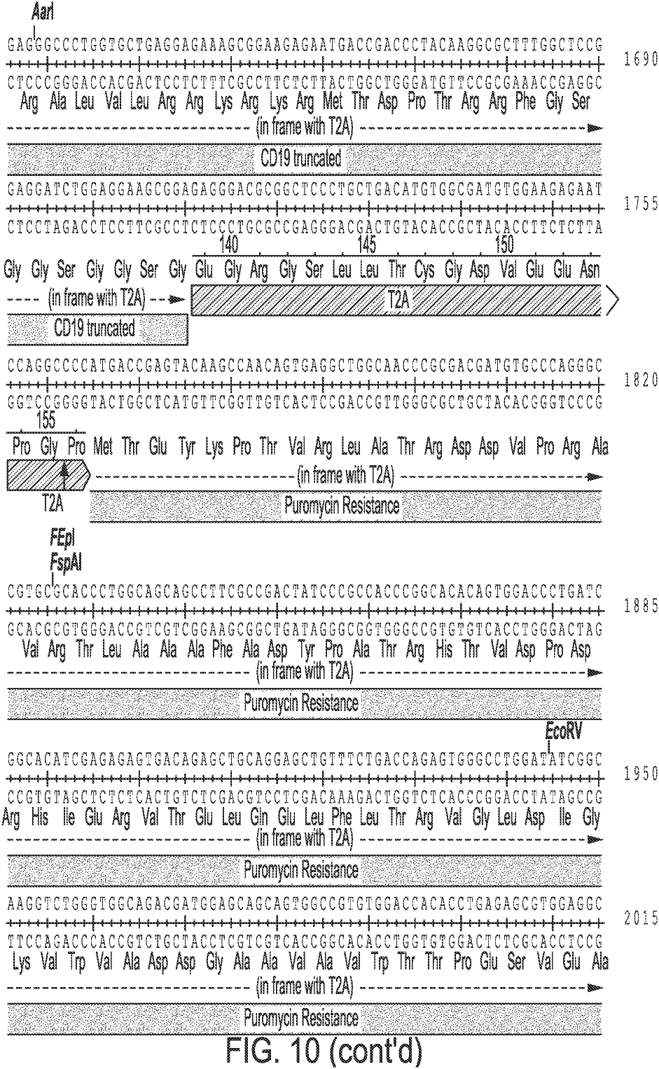

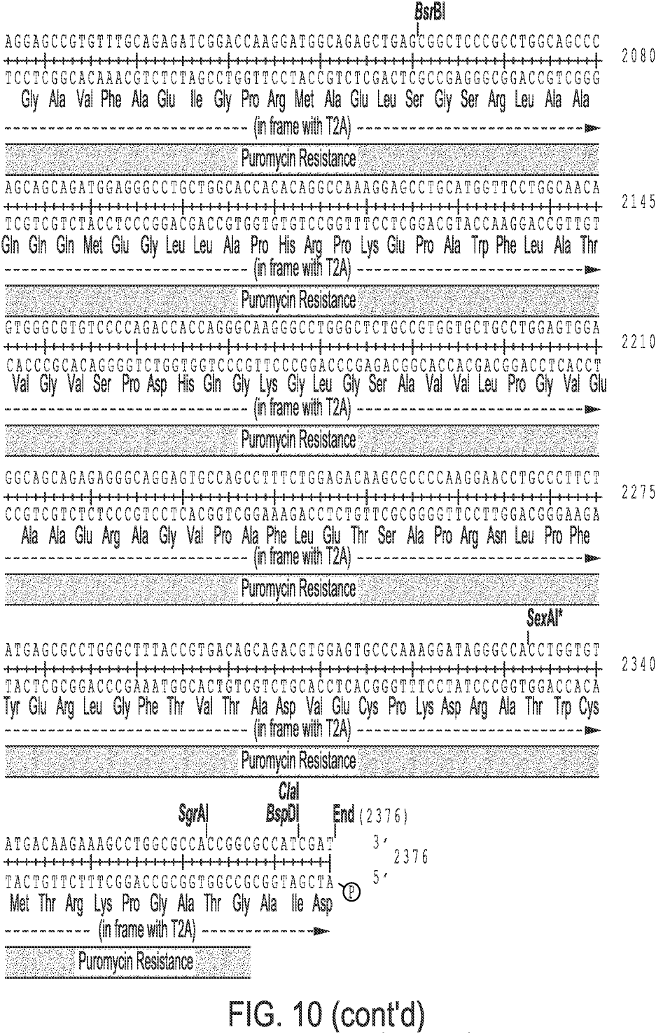

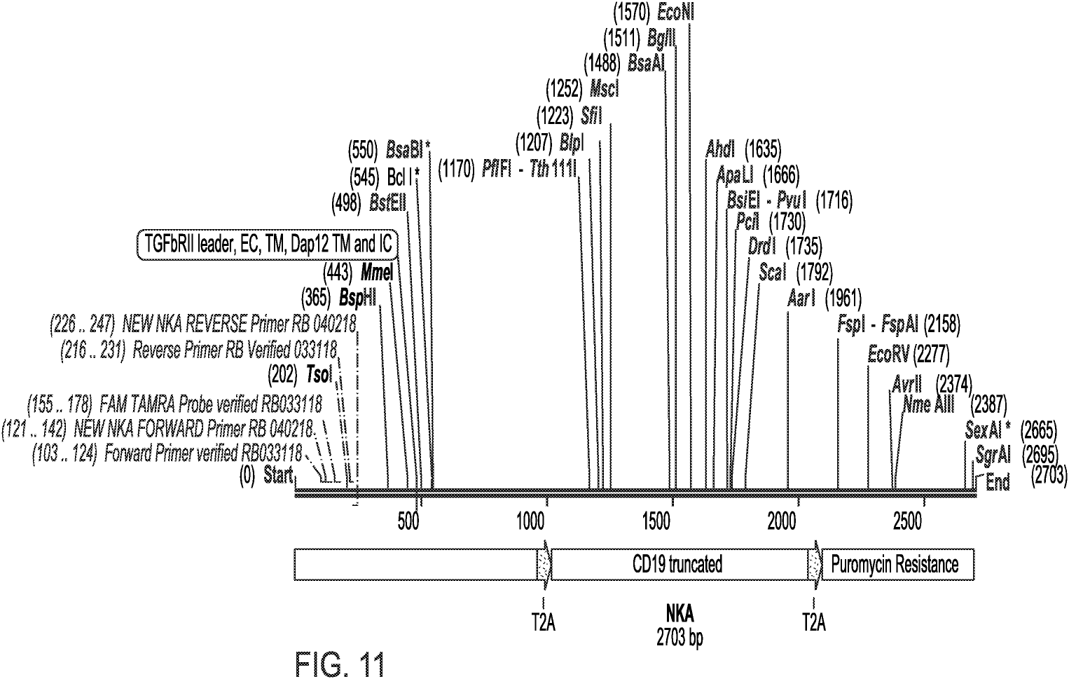

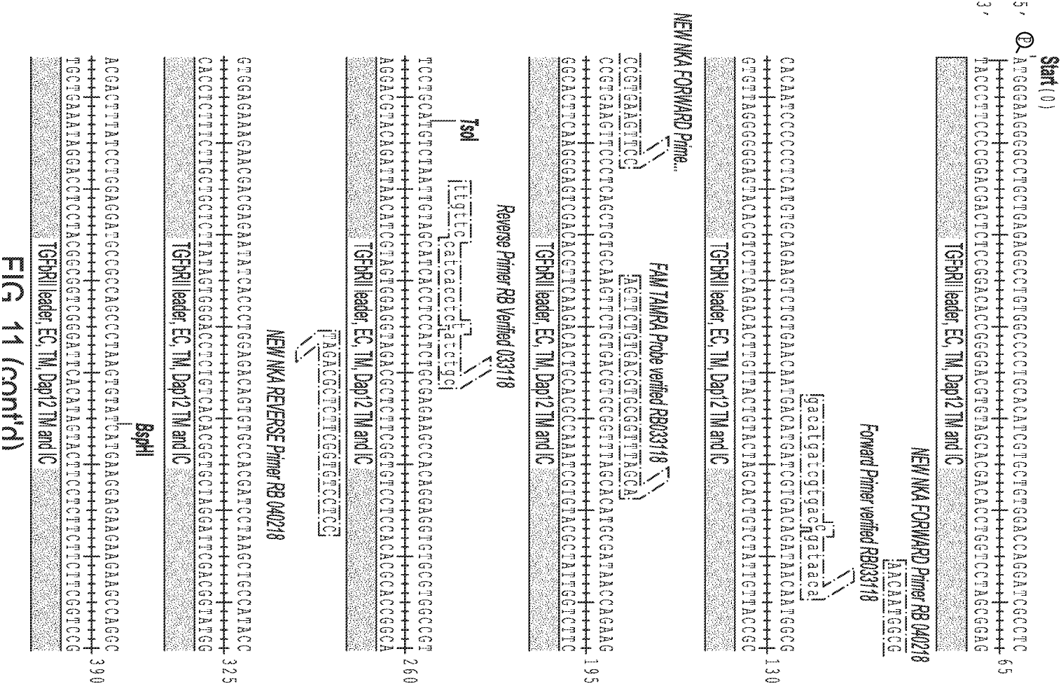

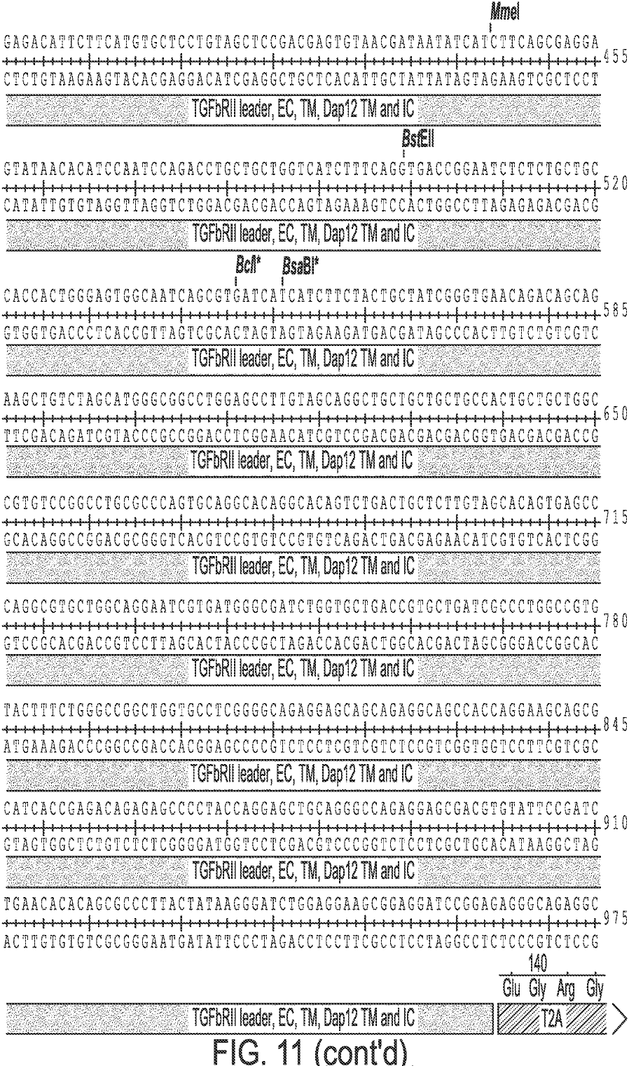

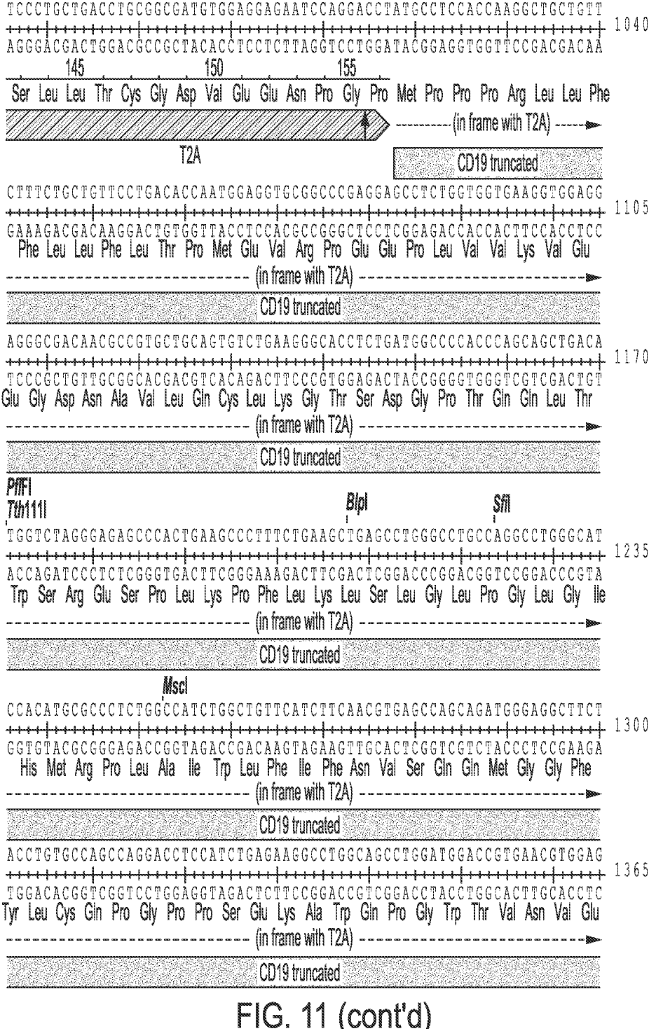

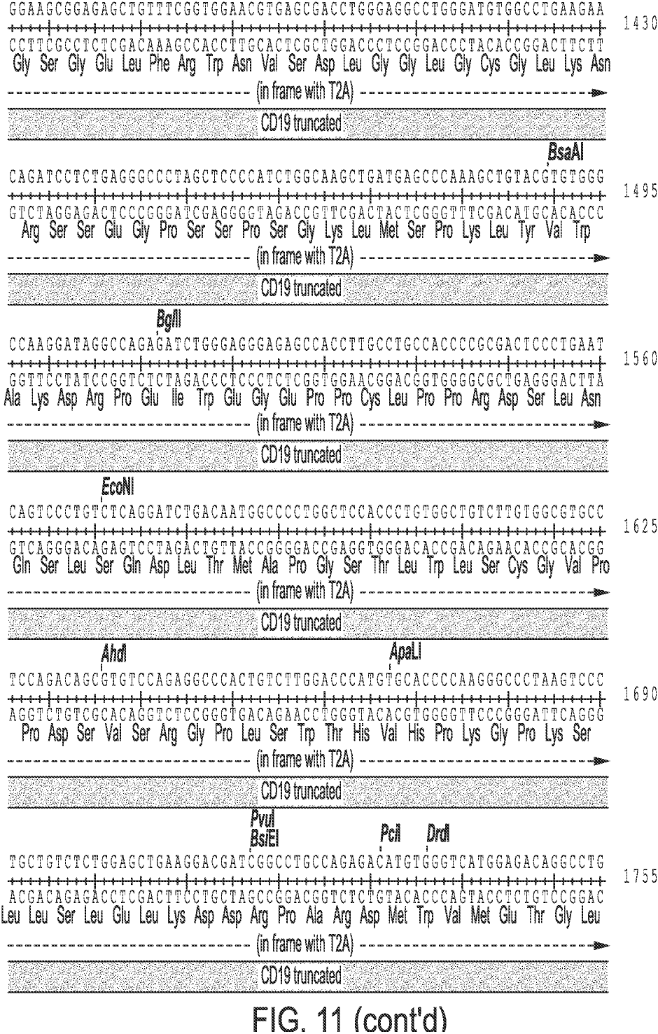

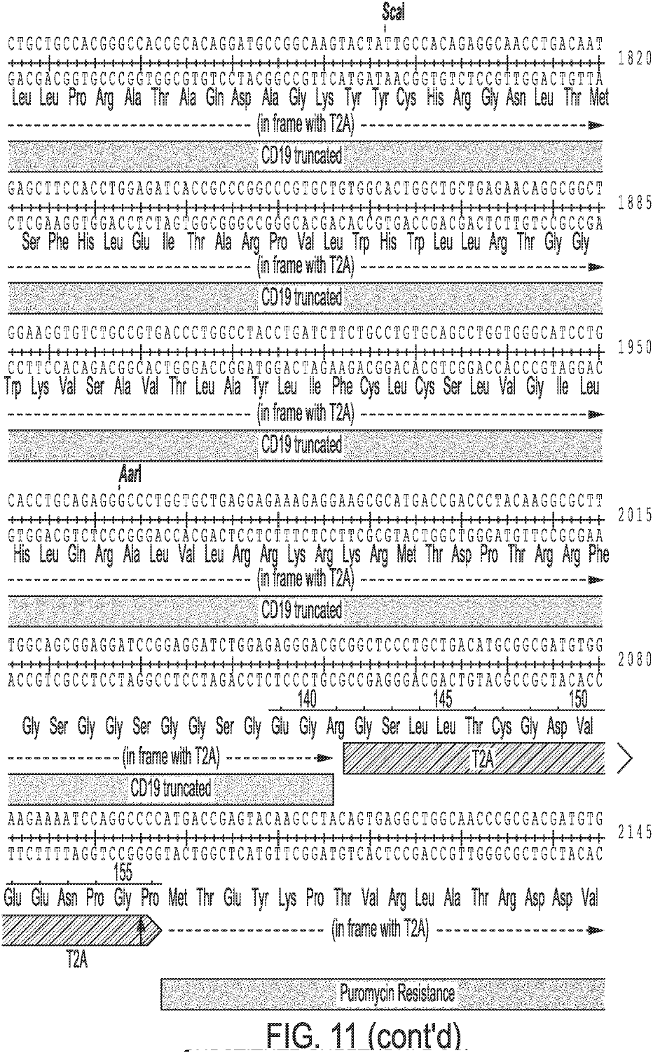

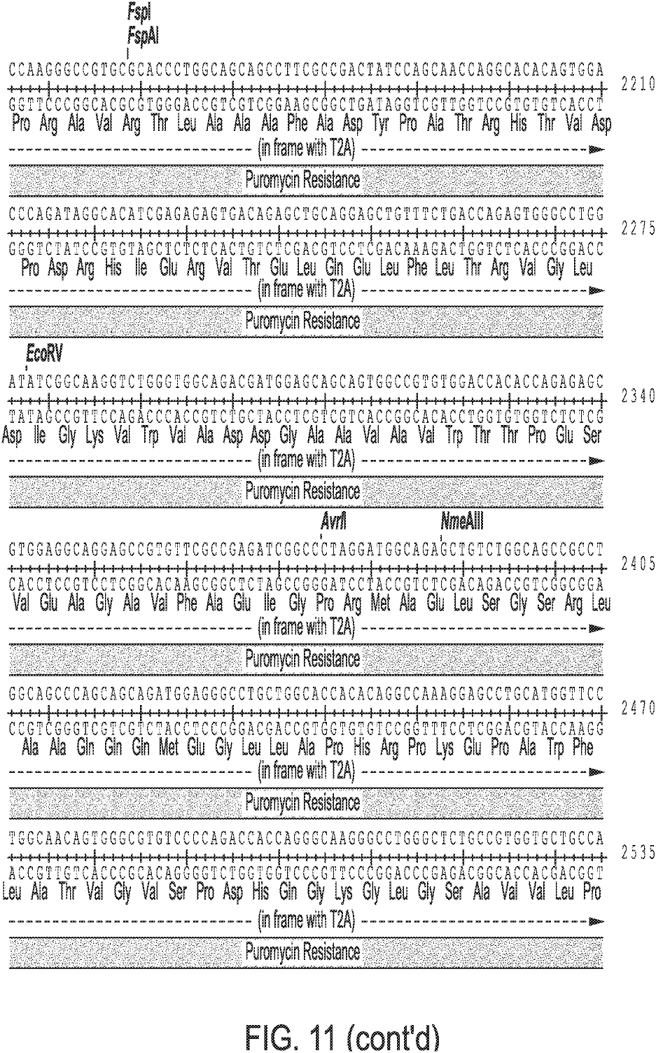

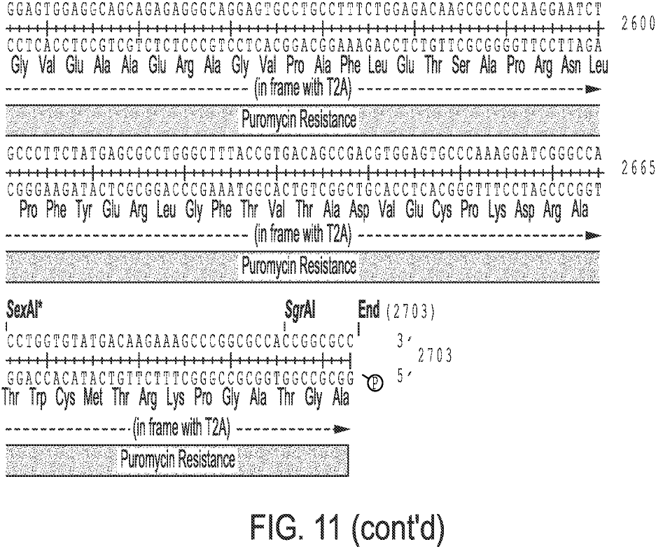

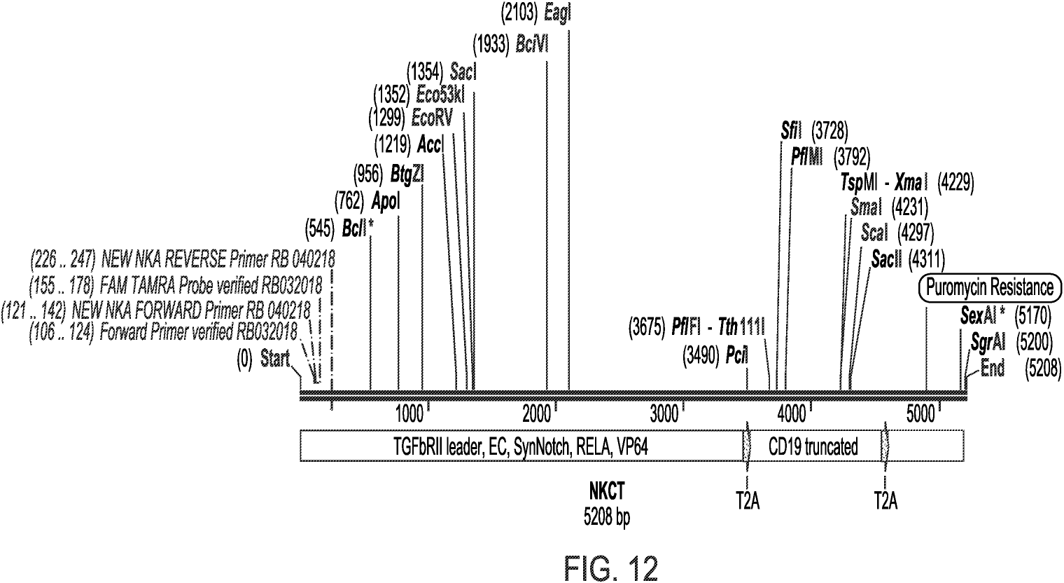

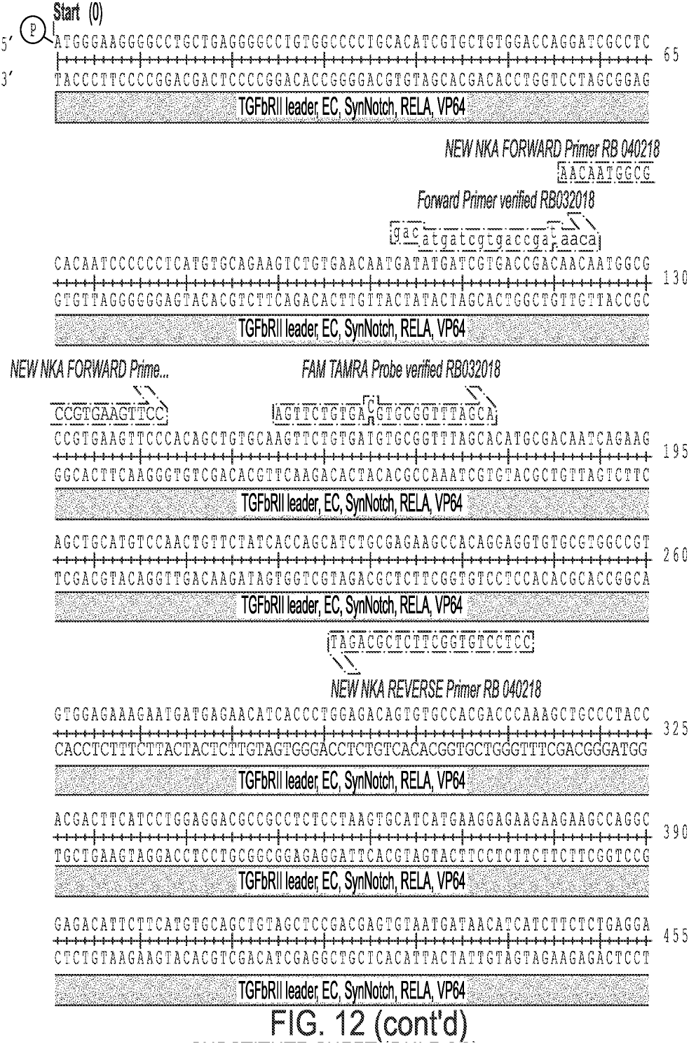

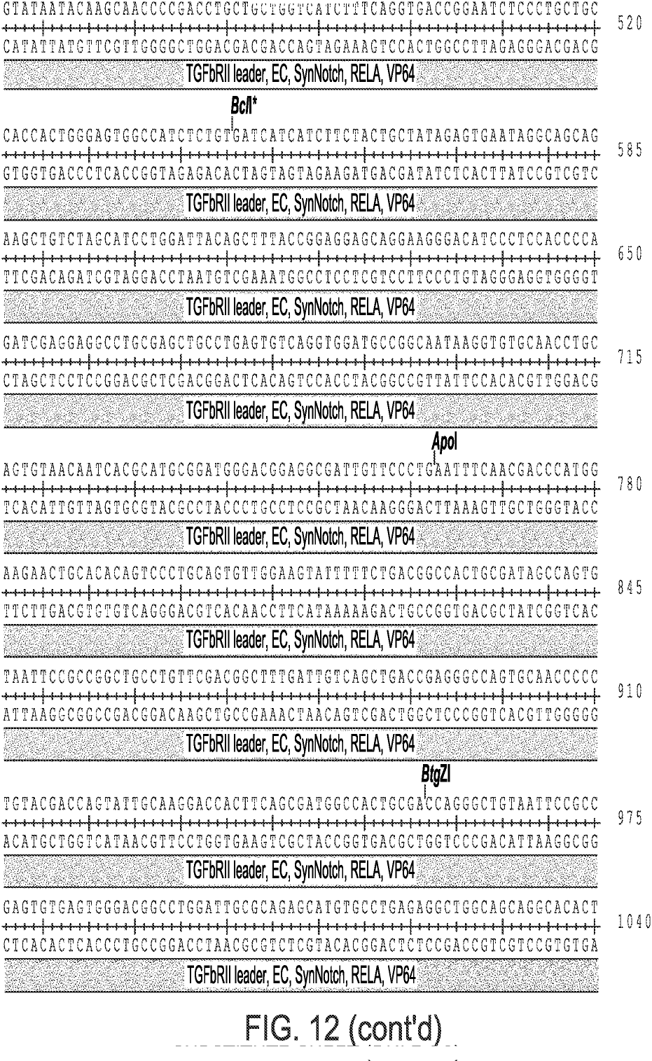

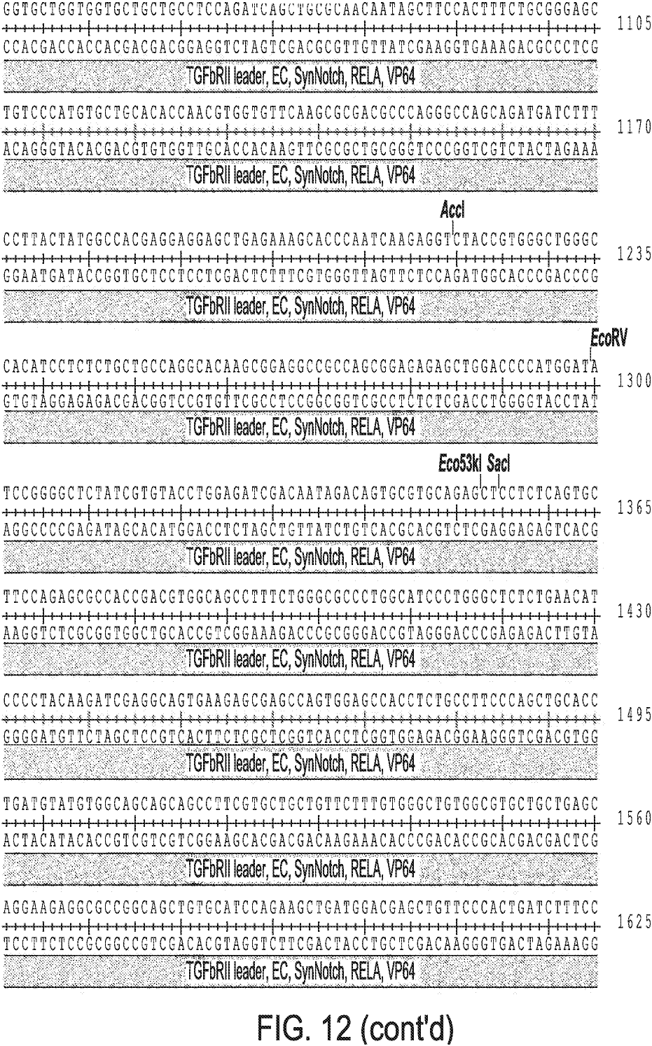

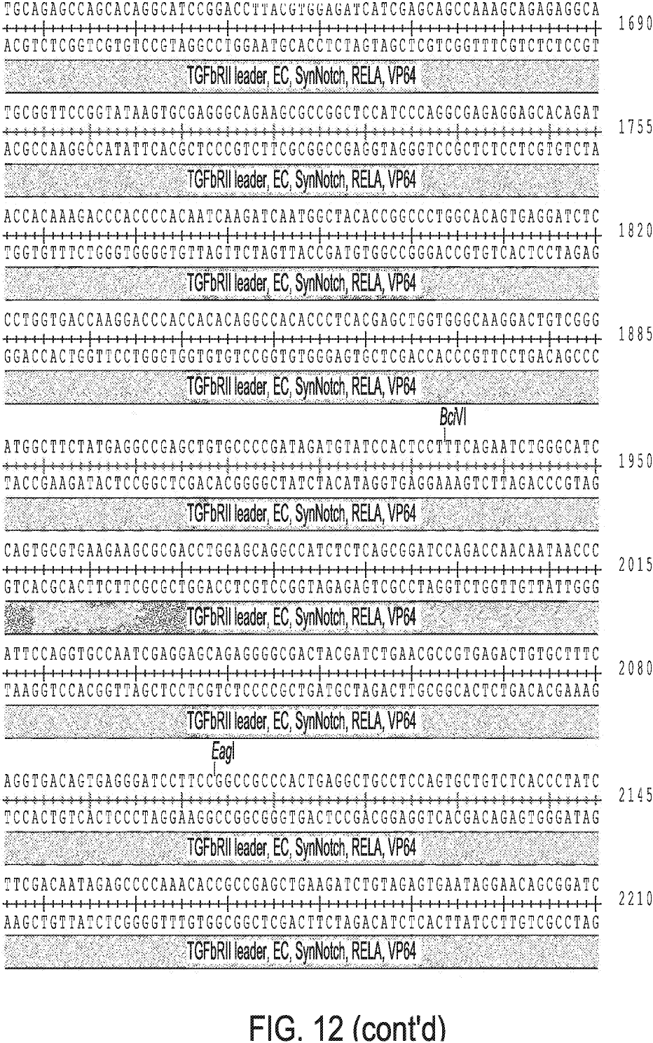

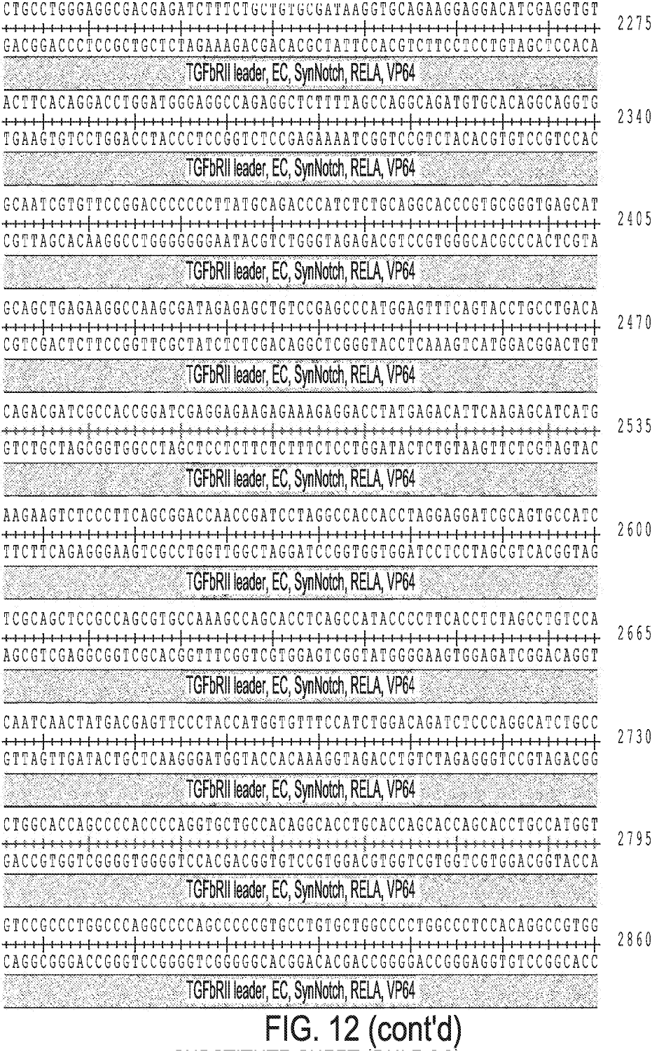

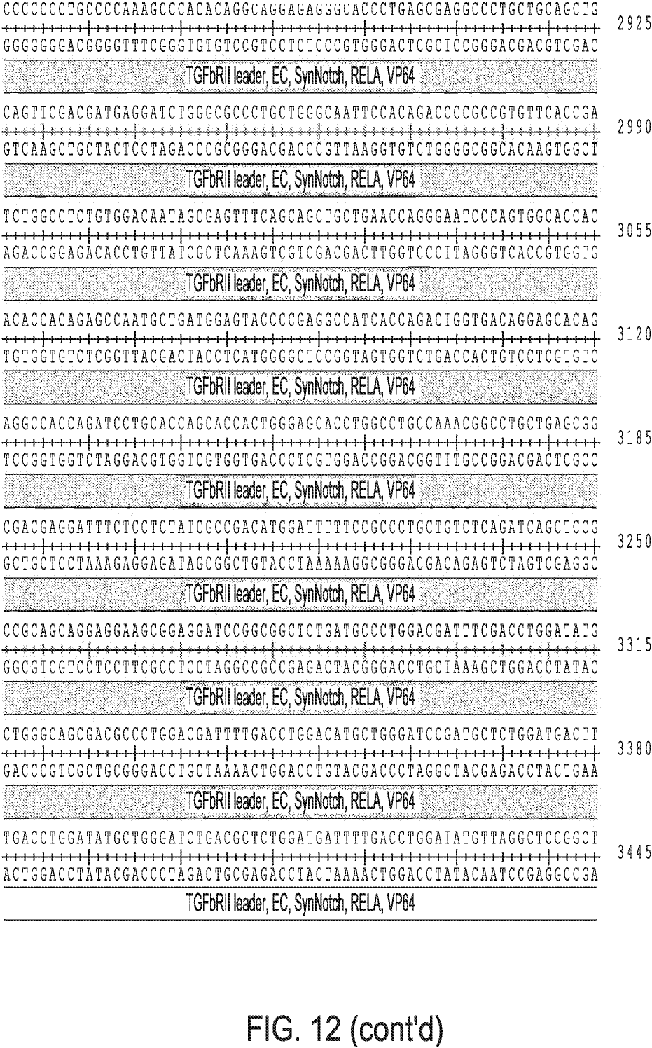

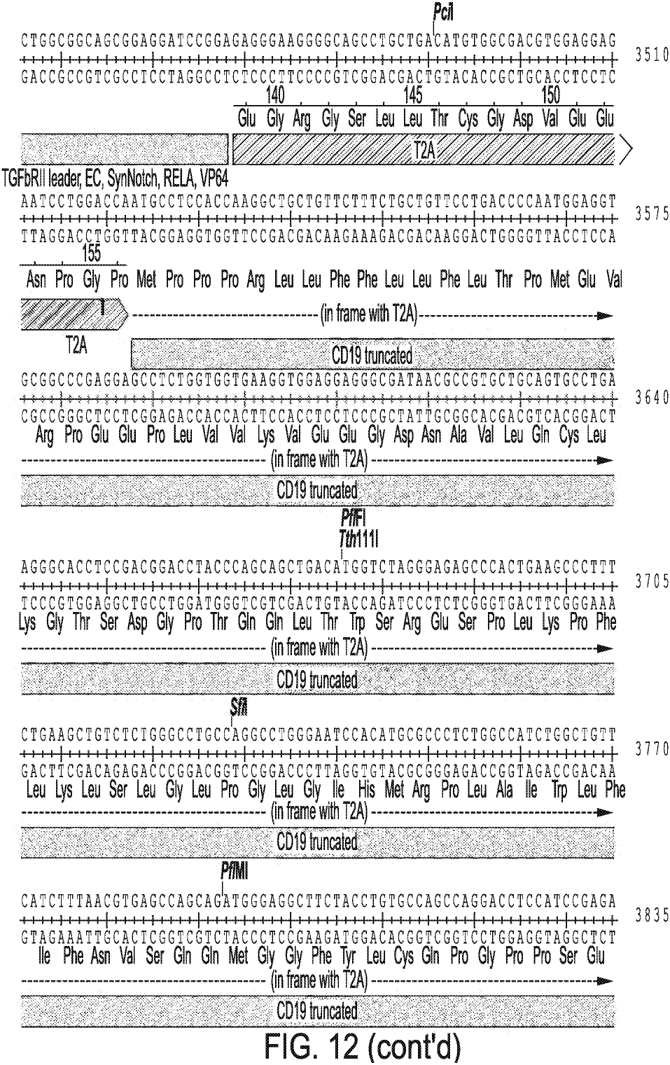

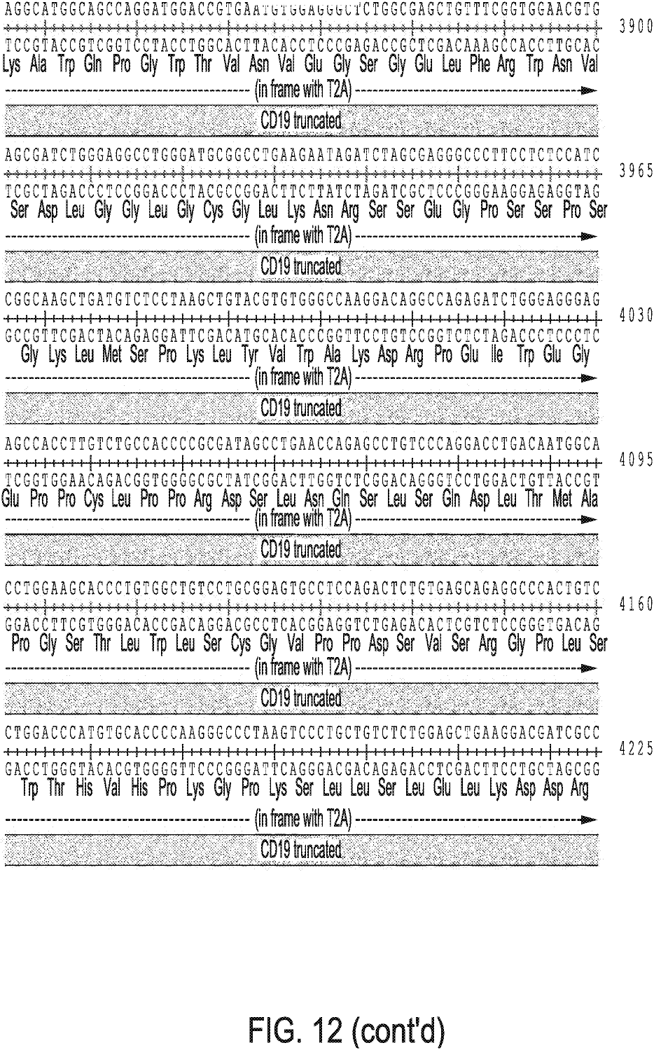

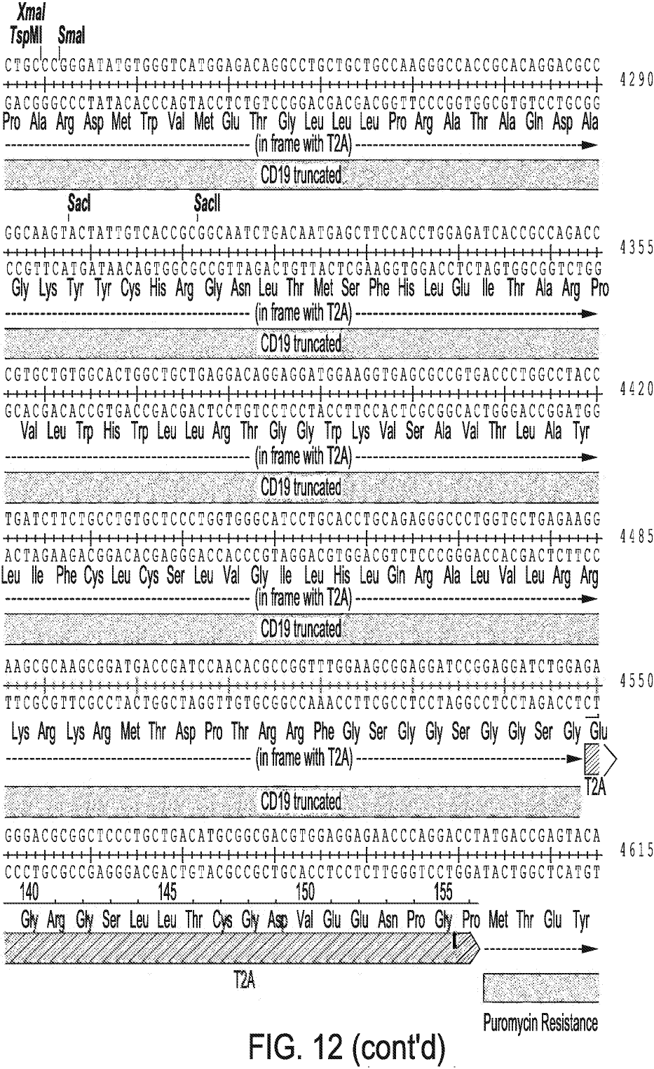

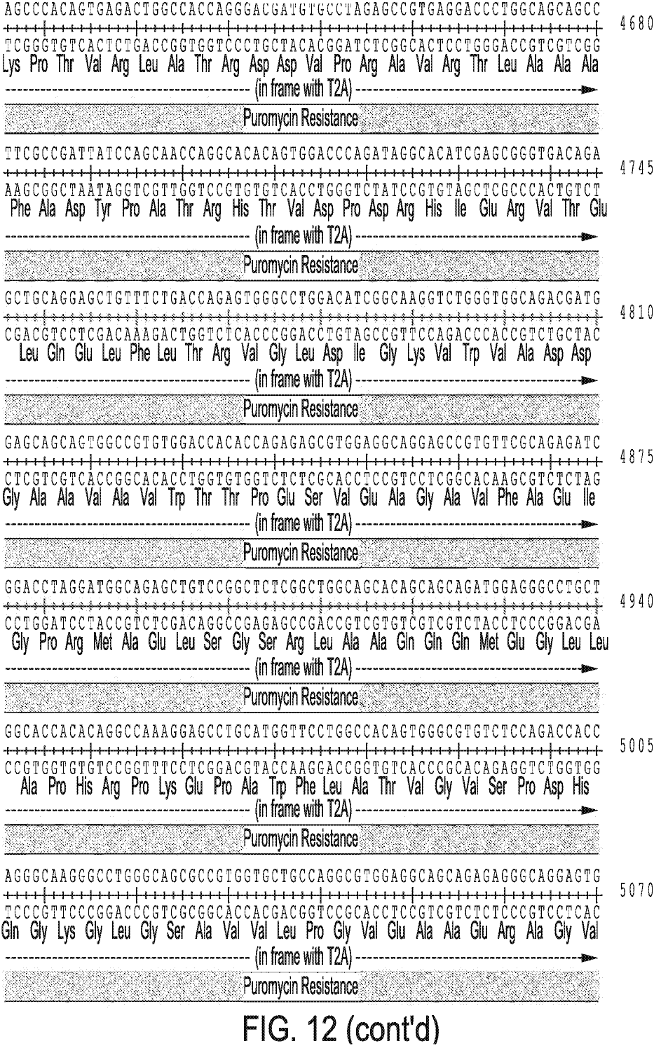

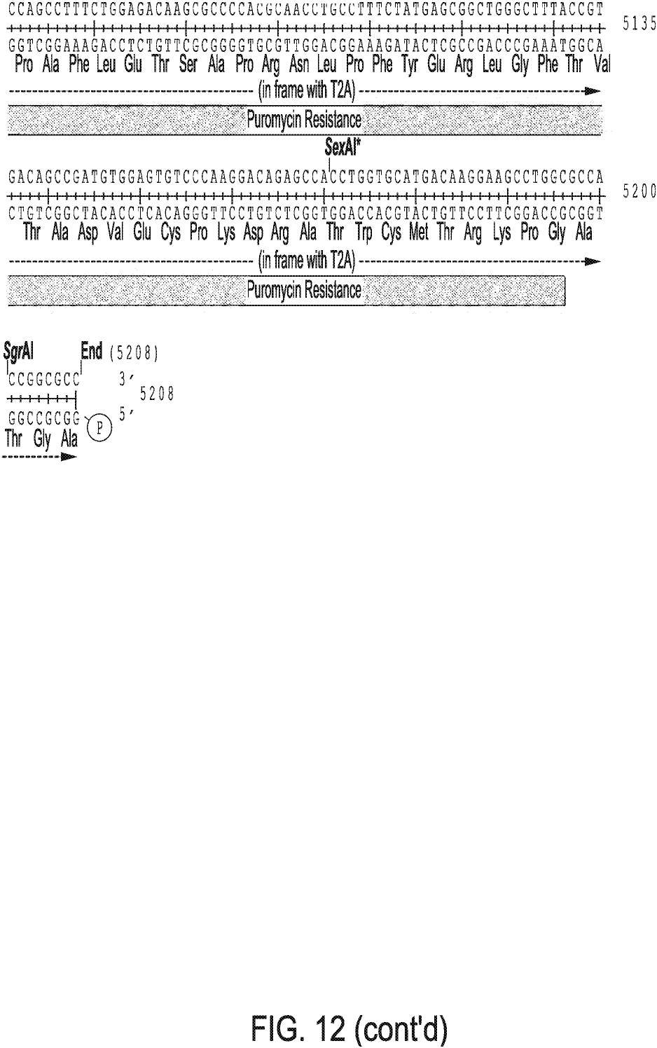

The same neuroblastoma xenograft model was established, as above, however animals were given repeat doses of untransduced or transduced NK cells on Days 0, 7, 15, 21, and 29 following tumor inoculation (FIG. 7A). As expected, bioluminescence quantification revealed that tumors rapidly progressed in untreated animals, with animals succumbing to their high tumor burden 1 month after inoculation (untreated median survival = 31 days, FIGS. 7B, 7C). With the repeat doses, animals treated with untransduced or mock-transduced NK cells were able to protect from tumor progression better than untreated animals, however these animals too succumbed to high tumor burden (UT median survival = 43 days. Mock-tdx median survival = 47 days, FIGS. 7B, 7C). In contrast, infusion of RBDNR or NKCT-transduced NK cells led to improved tumor control and prolongation of survival (progression free survival RBDNR = 25%, NKCT = 25%; survival untreated vs. RBDNR p=0.006, untreated vs. NKCT p=0.008; n=4 mice/group, FIG. 7D). Animals treated with NKA-transduced NK cells exhibited superior protection from tumor progression (FIGS. 7B, 7C) and significantly enhanced survival (progression free survival = 75%, survival untreated vs. NKA p=0.003, FIG. 7D). Taken together, these data suggest that, unlike their unmodified counterparts, NK cells modified to express novel variants of a TGFβ-receptor are able to protect from the inhibitory effects of neuroblastoma-associated TGFβ and demonstrate superior anti-tumor efficacy in vivo. Furthermore, coupling this TGFβ-receptor modification to the NK-specific signaling motif DAP12 may confer additional therapeutic advantages, as animals treated with the NKA-transduced NK cells achieved maximal survival and anti-tumor abilities in vitro and in vivo

The impact of TGFβ on the phosphorylation state of molecular signaling components as well as the expression of surface receptors is well-established^{1,32-36}, and the results provided herein supported the findings of the field. Phosphorylation of Smad2 and Smad3 was demonstrated as occurring as early as 30 minutes after TGFβ-exposure in unmodified NK cells, which we were able to prevent occurring in RBDNR, NKA, and NKCT-transduced NK cells. The signaling cascade initiated by the phosphorylation of Smad2/3 lead to impaired expression of surface receptors⁵³ and thus impaired anti-tumor cytolytic function. With the engineered receptors it was established herein that not only were cord-blood modified NK cells resistant to the inhibitory effects of tumor-associated TGFβ, but, in the case of the NKA receptor, they demonstrated superior anti-tumor functionality in the TGFβ-rich tumor setting. Although previous studies have demonstrated the appeal of rendering a cell therapeutic resistant to inhibitory TGFP^{37,43,54} in other disease models, this approach in neuroblastoma is unique. By incorporating activation domains to fully “hijack” the TGFβ receptor and convert an inhibitory signal to an ancillary signal, we established a novel “off the shelf” NK cell therapeutic.

The NKA receptor contains DAP12 fused to the truncated dominant negative receptor to facilitate NK-specific intracellular signaling, leading to improved activity in vivo. In native NK cells, DAP12 associates with natural activating and cytotoxicity receptors such as NKG2C and NKp44, and the ITAM-containing cytoplasmic domain can readily dock Zap70 and Syk proteins. Initiation of DAP12 activation signals for cell activation through the PI3K/ERK and Akt pathways. ^{45,55-60} By incorporating the transmembrane and ITAM-containing domains of DAP12 in the NKA construct, it was demonstrated that engagement of TGFβ with the engineered receptor triggered activation of DAP12 signaling and resulted in enhanced NK cell activity. Enhanced activity translated to increased anti-tumor efficacy, which lead to improved preclinical outcomes over strategies to singularly prevent TGFβ-mediated signaling. One other group has attempted to incorporate DAP12 signaling into prostate stem cell antigen (PSCA)-specific CAR construct, with preliminary results highlighting the benefit of the DAP12 construct over non-DAP12-containing CAR cells.⁶¹ It is the combination of the enhanced cell activity (with the DAP12 component) with the ameliorated suppressive effect (with the truncated dominant negative receptor component) that sets this product apart as an enhanced therapeutic.

The synthetic Notch receptor, which is incorporated into the NKCT receptor, is a strategy conceptualized and elegantly explored in the setting of chimeric antigen receptor generation for T cells.^{46.47} The concept employs logic gating by which a cell needs to receive a primary signal in order to trigger a secondary signal through a “SynNotch” receptor. This “SynNotch” receptor contains a core regulatory Notch domain, coupled to an intracellular transcriptional domain that is capable of cleaving and engaging with nuclear promoters to initiate a given transcriptional change ^{62,63} The NKCT receptor contains the extracellular TGFβ dominant negative receptor coupled to a Notch and RELA-linked domain; engagement of TGFβ with this receptor would trigger cleavage of the “SynNotch” motif which would lead to increased transcription of RELA (p65) and thus increased NK cell activation. Similar to the results as demonstrated with the NKA construct above, the in vitro observations appeared to validate the proposed method of action.

In this work, the SHSY5Y neuroblastoma line was used, which produced high levels of TGFβ in vivo from SHSY5Y-inoculated NSG mice. Although the engineered TGFβ receptors were able to protect against the impaired cytolytic activity of exogenous TGFβ as well as TGFβ-producing tumors in vitro, this protection was lost when TGFβ receptor-modified NK cells were placed in super-physiological (>50 ng/mL) environments. This is likely because the artificially high amount of TGFβ exceeded the saturation of the amount of modified TGFβ receptors on the NK cells. The dominant negative receptor strategy works by allowing the formation of the TGFβ receptor heterodimer between endogenous TGFβRl and the truncated (or modified) TGFβRII, and thus requires endogenous TGFβ receptors to be present on the cell.⁴² Because endogenous TGFβRII still exists on NK cells, in the setting of excess TGFβ, the surplus cytokine after binding to the engineered receptors is still able to bind endogenous receptors, which may in term negate the protective effect afforded by the engineered receptors. Selective knockdown of endogenous TGFβRII may address the situation, but does not represent a viable option therapeutically. An additional limitation pertains to the long-term receptor expression of genetically modified NK cells. These modified NK cells are identified based on changes in their expression of TGFβRII and expression of CD19 as compared to untransduced cells, but because these cells are co-expressing both components, there is the possibility that they selectively downregulate either the TGFβ modified receptor or the CD19 tag. By using immunomagnetic beads to selectively enrich the cell populations, we minimize the likelihood of this happening. Additionally, because a biological effect has been detected, and these cell populations can be identified after >4 weeks in vivo, it can be concluded that the engineered NK cells are likely maintaining expression of their modified TGFβ receptor long-term.

In summary, the cord blood-derived NK cells modified to protect from the inhibitory effects of TGFβ represent an efficient, fast-acting, innate therapeutic platform. In addition, the development of novel variant TGIβ receptor modifications described herein, composed of the dominant negative receptor coupled to intracellular signaling domains that can initiate NK cell activation, represents a unique cancer therapy that takes advantage of a tumor-abundant cytokine and converts a customarily inhibitory environment into a therapeutically advantageous environment. This strategy provides preclinical evidence to work towards the establishment of “off the shelf” gene-modified NK cells as a treatment modality for patients with neuroblastoma and other malignancies that utilize TGFβ secretion as a potent immune evasion mechanism.

### REFERENCES

1. Bottino C, Dondero A, Bellora F, et al. Natural killer cells and neuroblastoma: tumor recognition, escape mechanisms, and possible novel immunotherapeutic approaches. Front Immunol 2014;5:56.

2. Yang L. Pang Y, Moses HL. TGF-beta and immune cells: an important regulatory axis in the tumor microenvironment and progression. Trends Immunol 2010;31 :220-7.

3. Lee HM, Kim KS, Kim J. A comparative study of the effects of inhibitory cytokines on human natural killer cells and the mechanistic features of transforming growth factor-beta. Cell Immunol 2014;290:52-61 .

4. Meadows SK, Eriksson M, Barber A, Sentman CL. Human NK cell IFN-gamma production is regulated by endogenous TGF-beta. Int Immunopharmacol 2006;6:1020-8.

5. Cohen PS, Letterio JJ, Gaetano C, et al. Induction of transforming growth factor beta 1 and its receptors during all-trans-retinoic acid (RA) treatment of RA-responsive human neuroblastoma cell lines. Cancer Res 1995;55:2380-6.

6. Rouce RH, Shaim H, Sekine T, et al. The TGF-beta/SMAD pathway is an important mechanism for NK cell immune evasion in childhood B-acute lymphoblastic leukemia. Leukemia 2016;30:800-11.

7. Tarek N, Le Luduec JB, Gallagher MM, et al. Unlicensed NK cells target neuroblastoma following anti-GD2 antibody treatment. J Clin Invest 2012;122:3260-70.

8. Perica K, Varela JC, Oelke M, Schneck J. Adoptive T cell immunotherapy for cancer. Rambam Maimonides Med J 2015;6:e0004.

9. Rosenberg SA, Restifo NP, Yang JC, Morgan RA, Dudley M.E. Adoptive cell transfer: a clinical path to effective cancer immunotherapy Nat Rev Cancer 2008;8:299-308.

10. Rosenberg SA, Restifo NP. Adoptive cell transfer as personalized immunotherapy for human cancer. Science 2015;348:62-8.

11. Restifo NP, Dudley ME, Rosenberg SA. Adoptive immunotherapy for cancer: harnessing the T cell response. Nat Rev Immunol 2012;12:269-81.

12. Tey SK, Bollard CM, Heslop HE. Adoptive T-cell transfer in cancer immunotherapy. Immunol Cell Biol 2006;84:281-9.

13. Kalos M, June CH. Adoptive T cell transfer for cancer immunotherapy in the era of synthetic biology. Immunity 2013,39:49-60

14. Herberman RB, Nunn ME, Holden HT, Lavrin DH. Natural cytotoxic reactivity of mouse lymphoid cells against syngeneic and allogeneic tumors. II. Characterization of effector cells. Int J Cancer 1975;16:230-9.

15. Herberman RB, Nunn ME, Lavrin DH. Natural cytotoxic reactivity of mouse lymphoid cells against syngeneic acid allogeneic tumors. I. Distribution of reactivity and specificity. Int J Cancer 1975;16:216-29.

16. Kiessling R, Klein E, Pross H, Wigzell H. “Natural” killer cells in the mouse. II Cytotoxic cells with specificity for mouse Moloney leukemia cells. Characteristics of the killer cell. Eur J Immunol 1975;5:117-21.

17. Kiessling R, Klein E, Wigzell H. “Natural” killer cells in the mouse. I. Cytotoxic cells with specificity for mouse Moloney leukemia cells. Specificity and distribution according to genotype. Eur J Immunol 1975;5:112-7.

418. Wu J, Lanier LL. Natural killer cells and cancer. Advances in cancer research 2003,90:127-56.

19. Tam YK, Martinson J A, Doligosa K, Klingemann HG. Ex vivo expansion of the highly cytotoxic human natural killer-92 cell-line under current good manufacturing practice conditions for clinical adoptive cellular immunotherapy. Cytotherapy 2003;5:259-72.

20. Arai S, Meagher R, Swearingen M, et al. Infusion of the allogeneic cell line NK-92 in patients with advanced renal cell cancer or melanoma: a phase I trial. Cytotherapy 2008; 10:625-32.

21. Fujisaki H, Kakuda H, Shimasaki N, et al. Expansion of highly cytotoxic human natural killer cells for cancer cell therapy. Cancer Res 2009;69:4010-7.

22. Alici E, Sutlu T, Bjorkstrand B, et al. Autologous antitumor activity by NK cells expanded from myeloma patients using GMP-compliant components. Blood 2008;111 :3155-62.

23. Klingemann HG, Martinson J. Ex vivo expansion of natural killer cells for clinical applications. Cytotherapy 2004;6:15-22.

24. Lin SJ, Kuo ML. Cytotoxic function of umbilical cord blood natural killer cells: relevance to adoptive immunotherapy. Pediatr Hematol Oncol 2011;28:640-6.

25. Ruggeri L, Capanni M, Mancusi A, et al. Natural killer cell alloreactivity in haploidentical hematopoietic stem cell transplantation. Int J Hematol 2005;81:13-7

26. Velardi A, Ruggeri L, Mancusi A, Aversa F, Christiansen FT. Natural killer cell allorecognition of missing self in allogeneic hematopoietic transplantation: a tool for immunotherapy of leukemia. Curr Opin Immunol 2009;21:525-30.

27. Velardi A. Role of KIRs and KIR ligands in hematopoietic transplantation. Curr Opin Immunol 2008;20:581-7.

28. Shah N, Martin-Antonio B, Yang H, et al. Antigen presenting cell-mediated expansion of human umbilical cord blood yields log-scale expansion of natural killer cells with anti-myeloma activity. PLoS One 2013;8:e76781.

29. Gluckman E. Milestones in umbilical cord blood transplantation. Blood reviews 2011;25:255-9.

30. Kanold J, Paillard C, Tchirkov A, et al. NK cell immunotherapy for high-risk neuroblastoma relapse after haploidentical HSCT. Pediatric blood & cancer 2012;59:739-42.

3 1. Heldin CH, Miyazono K, ten Dijke P. TGF-beta signalling from cell membrane to nucleus through SMAD proteins. Nature 1997;390:465-71.

32 Rook AH, Kehrl JH, Wakefield LM, et al. Effects of transforming growth factor beta on the functions of natural killer cells: depressed cytolytic activity and blunting of interferon responsiveness. J Immunol 1986;136:3916-20.

33. Sun C, Fu B, Gao Y, et al. TGF-beta1 down-regulation of NKG2D/DAP10 and 2B4/SAP expression on human NK cells contributes to HBV persistence. PLoS Pathog 2012;8:e1002594.

34. Crane CA, Han SJ, Barry JJ, Ahn BJ, Lanier LL, Parsa AT. TGF-beta downregulates the activating receptor NKG2D on NK cells and CD8+ T cells in glioma patients. Neuro Oncol 2010;12:7-13.

35. Guo SW, Du Y, Liu X. Platelet-derived TGF-beta1 mediates the down-modulation of NKG2D expression and may be responsible for impaired natural killer (NK) cytotoxicity in women with endometriosis. Hum Reprod 2016;31:1462-74.

36. Scarpa S, Coppa A, Ragano-Caracciolo M, et al. Transforming growth factor beta regulates differentiation and proliferation of human neuroblastoma. Exp Cell Res 1996;229:147-54.

37 Yvon ES, Burga R, Powell A, et al. Cord blood natural killer cells expressing a dominant negative TGF-beta receptor: Implications for adoptive immunotherapy for glioblastoma. Cytotherapy 2017;19:408-18.

38. Chen RH, Ebner R, Derynck R Inactivation of the type II receptor reveals two receptor pathways for the diverse TGF-beta activities. Science 1993;260:1335-8.

39. Brand T, MacLellan WR, Schneider MD. A dominant-negative receptor for type beta transforming growth factors created by deletion of the kinase domain. J Biol Chem 1993;268:11500-3.

40. Lacuesta K, Buza E, Hauser H, et al. Assessing the safety of cytotoxic T lymphocytes transduced with a dominant negative transforming growth factor-beta receptor. J Immunother 2006;29:250-60.

41. Foster AE, Dotti G, Lu A, et al. Antitumor activity of EBV-specific T lymphocytes transduced with a dominant negative TGF-beta receptor. J Immunother 2008;31:500-5.

42. Wieser R, Attisano L, Wrana JL, Massague J. Signaling activity of transforming growth factor beta type II receptors lacking specific domains in the cytoplasmic region. Mol Cell Biol 1993;13:7239-47.

43. Kloss CC, Lee J, Zhang A, et al. Dominant-Negative TGF-beta Receptor Enhances PSMA-Targeted Human CAR T Cell Proliferation And Augments Prostate Cancer Eradication. Mol Ther 2018.

44. McVicar DW, Taylor LS, Gosselin P, et al. DAP12-mediated signal transduction in natural killer cells. A dominant role for the Syk protein-tyrosine kinase. J Biol Chem 1998;273:32934-42.

45. Turnbull IR, Colonna M. Activating and inhibitory functions of DAP12. Nat Rev Immunol 2007;7:155-61.

46. Morsut L, Roybal KT, Xiong X, et al. Engineering Customized Cell Sensing and Response Behaviors Using Synthetic Notch Receptors. Cell 2016;164:780-91.

47. Roybal KT, Williams JZ, Morsut L, et al. Engineering T Cells with Customized Therapeutic Response Programs Using Synthetic Notch Receptors. Cell 2016;167:419-32 e16.

48. Cho D, Campana D. Expansion and activation of natural killer cells for cancer immunotherapy. Korean J Lab Med 2009;29:89-96

49. Kovalevich J, Langford D. Considerations for the use of SH-SY5Y neuroblastoma cells in neurobiology. Methods Mol Biol 2013;1078:9-21.

50. Liu E, Tong Y, Dotti G, et al. Cord blood NK cells engineered to express IL-15 and a CD19-targeted CAR show long-term persistence and potent antitumor activity. Leukemia 2018;32:520-31.

51. Lim E, Modi KD, Kim J. In vivo bioluminescent imaging of mammary tumors using IVIS spectrum. J Vis Exp 2009.

52. Kim JB, Urban K, Cochran E, et al. Non-invasive detection of a small number of bioluminescent cancer cells in vivo. PLoS One 2010;5:e9364.

53. Tran HC, Wan Z, Sheard MA, et al. TGFbetaR1 Blockade with Galunisertib (LY2157299) Enhances Anti-Neuroblastoma Activity of the Anti-GD2 Antibody Dinutuximab (ch14.18) with Natural Killer Cells. Clin Cancer Res 2017;23:804-13.

54. Yang B, Liu H, Shi W, et al. Blocking transforming growth factor-beta signaling pathway augments antitumor effect of adoptive NK-92 cell therapy. Int Immunopharmacol 2013;17:198-204.

55. Wei P, Xu L, Li CD, et al. Molecular dynamic simulation of the self-assembly of DAP 12-NKG2C activating immunoreceptor complex. PLoS One 20 14;9:e105560.

56. Lanier LL, Bakker AB. The ITAM-bearing transmembrane adaptor DAP12 in lymphoid and myeloid cell function. Immunol Today 2000;21:611-4.

57. Lanier LL, Corliss B, Wu J, Phillips JH. Association of DAP12 with activating CD94/NKG2C NK cell receptors. Immunity 1998;8:693-701.

58. Campbell KS, Yusa S, Kikuchi-Maki A, Catina TL. NKp44 triggers NK cell activation through DAP12 association that is not influenced by a putative cytoplasmic inhibitory sequence. J Immunol 2004;172:899-906.

59. Campbell KS, Colonna M. DAP12: a key accessory protein for relaying signals by natural killer cell receptors. Int J Biochem Cell Biol 1999;31 :631-6.

60. Winter JN, Jefferson LS, Kimball SR. ERK and Akt signaling pathways function through parallel mechanisms to promote mTORC1 signaling Am J Physiol Cell Physiol 2011;304:C1172-80.

61. Topfer K, Cartellieri M, Michen S, et al. DAP12-based activating chimeric antigen receptor for NK cell tumor immunotherapy. J Immunol 2015;194:3201-12.

62. Bray SJ. Notch signalling: a simple pathway becomes complex. Nat Rev Mol Cell Biol 2006;7:678-89.

63. Selkoe D, Kopan R. Notch and Presenilin: regulated intramembrane proteolysis links development and degeneration. Annu Rev Neurosci 2003;26:565-97.

64. Wild J, Schmiedel BJ, Maurer A, et al. Neutralization of (NK-cell-derived) B-cell activating factor by Belimumab restores sensitivity of chronic lymphoid leukemia cells to direct and Rituximab-induced NK. lysis. Leukemia 2015;29:1676-83.

65. Schleinitz N, Vely F, Harle JR, Vivier E. Natural killer cells in human autoimmune diseases. Immunology 2010;131:451-8.

66. Lapteva N, Parihar R, Rollins LA, Gee AP, Rooney CM. Large-Scale Culture and Genetic Modification of Human Natural Killer Cells for Cellular Therapy. Methods Mol Biol 2016;1441:195-202.

67. Lapteva N, Szmania SM, van Rhee F, Rooney CM. Clinical grade purification and expansion of natural killer cells. Crit Rev Oncog 2014;19:121-32.

68. Bollard CM, Rossig C, Calonge MJ, et al. Adapting a transforming growth factor beta-related tumor protection strategy to enhance antitumor immunity. Blood 2002;99:3179-87.

69. Zalatan JG, Lee ME, Almeida R, et al. Engineering complex synthetic transcriptional programs with CRISPR RNA scaffolds. Cell 2015; 160:339-50.

70. Wagner HJ, Bollard CM, Vigouroux S, et al. A strategy for treatment of Epstein-Barr virus-positive Hodgkin’s disease by targeting interleukin 12 to the tumor environment using tumor antigen-specific T cells. Cancer Gene Ther 2004;11:81-91.

Example 8

New Modified Nulceic Acid Constructs

| SEQ ID | DESCRIPTION |
|---|---|
| 1 | T2A peptide |
| 2 | ΔCD 19 |
| 3 | Leader Sequence |
| 4 | TGFβ-RII ECD |
| 5 | TGFβ-RII TMD |
| 6 | TGFβ-RII ΔICD |
| 7 | DNAX-activation protein 12 ECD |
| 8 | DNAX-activation protein 12 TMD |
| 9 | DNAX-activation protein 12 ICD |
| 10 | KIR2DS2 ΔECD |
| 11 | KIR2DS2 TMD |
| 12 | KIR2DS2 ICD |
| 13 | NKA |
| 14 | NKA2 |
| 15 | NKA3 |
| 16 | SFG-NKA2 |

Schematic of the structure of the retroviral vector SFG encoding the NKA, NKA2, and NKA3 receptors:

NKA

| | | | |
|---|---|---|---|
| ΔTGFβ-RII | DAP12 | T2A | ΔCD19 |

NKA2

| | | | |
|---|---|---|---|
| ΔTGFβ-RII | DAP12 | T2A | ΔCD19 |

NKA3

| | | | |
|---|---|---|---|
| ΔTGFβ-RII | ΔKIR2DS2 | T2A | ΔCD19 |

<210> SEQ ID NO 1
<211> LENGTH: 54
<212> TYPE: DNA
<213> ORGANISM: Thosea asigna virus
<400> Sequence: 1
GAGGGCAGAG GCTCCCTGCT GACCTGCGGC GATGTGGAGG AGAATCCAGG ACCT 54
<210> SEQ ID NO 2
<211> LENGTH: 999
<212> TYPE: DNA
<213> ORGANISM: Home sapiens
<400> Sequence: 2
ATGCCTCCAC CAAGGCTGCT GTTCTTTCTG CTGTTCCTGA CACCAATGGA GGTGCGGCCC 60
GAGGAGCCTC TGGTGGTGAA GGTGGAGGAG GGCGACAACG CCGTGCTGCA GTGTCTGAAG 120
GGCACCTCTG ATGGCCCCAC CCAGCAGCTG ACATGGTCTA GGGAGAGCCC ACTGAAGCCC 180
TTTCTGAAGC TGAGCCTGGG CCTGCCAGGC CTGGGTATCC ACATGCGCCC TCTGGCCATC 240
TGGCTGTTCA TCTTCAACGT GAGCCAGCAG ATGGGAGGCT TCTACCTGTG CCAGCCAGGA 300
CCTCCATCTG AGAAGGCCTG GCAGCCTGGA TGGACCGTGA ACGTGGAGGG AAGCGGAGAG 360
CTGTTTCGGT GGAACGTGAG CGACCTGGGA GGCCTGGGAT GTGGCCTGAA GAACAGATCC 420
TCTGAGGGCC CTAGCTCCCC ATCTGGCAAG CTGATGAGCC CAAAGCTGTA CGTGTGGGCC 480
AAGGATAGGC CAGAGATCTG GGAGGGAGAG CCACCTTGCC TGCCACCCCG CGACTCCCTG 540
AATCAGTCCC TGTCTCAGGA TCTGACAATG GCCCCTGGCT CCACCCTGTG GCTGTCTTGT 600
GGCGTGCCTC CAGACAGCGT GTCCAGAGGC CCACTGTCTT GGACCCACGT GCACCCCAAG 660
GGCCCTAAGT CCCTGCTGTC TCTGGAGCTG AAGGACGATC GGCCTGCCAG AGACATGTGG 720
GTCATGGAGA CAGGCCTGCT GCTGCCACGG GCCACCGCAC AGGATGCCGG CAAGTACTAT 780
TGCCACAGAG GCAACCTGAC AATGAGCTTC CACCTGGAGA TCACCGCCCG GCCCGTGCTG 840
TGGCACTGGC TGCTGAGAAC AGGCGGCTGG AAGGTGTCTG CCGTGACCCT GGCCTACCTG 900
ATCTTCTGCC TGTGCAGCCT GGTGGGCATC CTGCACCTGC AGAGGGCCCT GGTGCTGAGG 960
AGAAAGAGGA AGCGCATGAC CGACCCTACA AGGCGCTTT 999
<210> SEQ ID NO 3
<211> LENGTH: 66
<212> TYPE: DNA
<213> ORGANISM: Home sapiens
<400> Sequence: 3
ATGGGAAGGG GCCTGCTGAG AGGCCTGTGG CCCCTGCACA TCGTGCTGTG GACCAGGATC 60
GCCTCC 66
<210> SEQ ID NO 4
<211> LENGTH: 432
<212> TYPE: DNA
<213> ORGANISM: Home sapiens
<400> Sequence: 4
ACAATCCCCC CTCATGTGCA GAAGTCTGTG AACAATGACA TGATCGTGAC AGATAACAAT 60
GGCGCCGTGA AGTTCCCTCA GCTGTGCAAG TTCTGTGACG TGCGGTTTAG CACATGCGAT 120
AACCAGAAGT CCTGCATGTC TAATTGTAGC ATCACCTCCA TCTGCRAGAA GCCACAGGAG 180
GTGTGCGTGG CCGTGTGGAG AAAGAACGAC GAGAATATCA CCCTGGAGAC AGTGTGCCAC 240
GATCCTAAGC TGCCATACCA CGACTTTATC CTGGAGGATG CCGCCAGCCC TAAGTGTATC 300
ATGAAGGAGA AGAAGAAGCC AGGCGAGACA TTCTTCATGT GCTCCTGTAG CTCCGAGGAG 360
TGTAACGATA ATATCATCTT CAGCGAGGAG TATAACACAT CCAATCCAGA CCTGCTGCTG 420
GTCATCTTTC AG 432
<210> SEQ ID NO 5
<211> LENGTH:
<212> TYPE: DNA
<213> ORGANISM: Home sapiens
<400> Sequence: 5
GTGACAGGCA TCAGCCTCCT GCCACCACTG GGAGTTGCCA TATCTGTCAT CATCATCTTC 60
TAC 63
<210> SEQ ID NO 6
<211.> LENGTH: 36
<212> TYPE: DNA
<213> ORGANISM: Home sapiens
<400> Sequence: 6
TGCTACCGCG TTAACCGGCA GCAGAAGCTG AGTTCA 36
<210> SEQ ID NO 7
<211> LENGTH: 57
<212> TYPE: DNA
<213> ORGANISM: Home sapiens
<400> Sequence: 7
CTGCGCCCAG TGCAGGCACA GGCACAGTCT GACTGCTCTT GTAGCACAGT GAGCCCA 57
<210> SEQ ID NO 8
<211> LENGTH: 63
<212> TYPE: DNA
<213> ORGANISM: Home sapiens
<400> Sequence: 8
GGCGTGCTGG CAGGAATCGT GATGGGCGAT CTGGTGCTGA CCGTGCTGAT CGCCCTGGCC 60
GTG 63
<210> SEQ ID NO 9
<211> LENGTH: 156
<212> TYPE: DNA
<213> ORGANISM: Home sapiens
<400> Sequence: 9
TACTTTCTGG GCCGGCTGGT GCCTCGGGGC AGAGGAGCAG CAGAGGCAGC CACCAGGAAG 60
CAGCGCATCA CCGAGACAGA GAGCCCCTAC CAGGAGCTGC AGGGCCAGAG GAGCGACGTG 120
TATTCCGATC TGAACACACA GCGCCCTTAC TATAAG 156
<210> SEQ ID NO 10
<211> LENGTH: 48
<212> TYPE: DNA
<213> ORGANISM: Home sapiens
<400> Sequence: 10
TCACCCACTG AACCAAGCTC CAAAACCGGT AACCCCAGAC ACCTGCAT 48
<210> SEQ ID NO 11
<2.11> LENGTH: 63
<212> TYPE: DNA
<213> ORGANISM: Home sapiens
<400> Sequence: 11
GGCGTGCTGG CAGGAA-ICGT GATGGGCGAT CTGGTGCTGA CCGTGCTGAT CGCCCTGGCC 60
GTG 63
<210> SEQ ID NO 12
<211> LENGTH: 156
<212> TYPE: DNA
<213> ORGANISM: Home sapiens
<400> Sequence: 12
TACTTTCTGG GCCGGCTGGT GCCTCGGGGC AGAGGAGCAG CAGAGGCAGC CACCAGGAAG 60
CAGCGCATCA CCGAGACAGA GAGCCCCTAC CAGGAGCTGC AGGGCCAGAG GAGCGACGTG 120
TATTCCGATC TGAACACACA GCGCCCTTAC TATAAG 156
<210> SEQ ID NO 13
<211> LENGTH: 2016
<212> TYPE: DNA
<213> ORGANISM: N/A
<400> Sequence: 13
ATGGGAAGGG GCCTGCTGAG AGGCCTGTGG CCCCTGCACA TCGTGCTGTG GACCAGGATC 60
GCCTCCACAA TCCCCCCTCA TGTGCAGAAG TCTGTGAACA ATGACATGAT CGTGACAGAT 120
AACAATGGCG CCGTGAAGTT CCCTCAGCTG TGCAAGTTCT GTGACGTGCG GTTTAGCACA 180
TGCGATAACC AGAAGTCCTG CATGTCTAAT TGTAGCATCA CCTCCATCTG CGAGAAGCCA 240
CAGGAGGTGT GCGTGGCCGT GTGGAGAAAG AACGACGAGA ATATCACCCT GGAGACAGTG 300
TGCCACGATC CTAAGCTGCC ATACCACGAC TTTATCCTGG AGGATGCCGC CAGCCCTAAG 360
TGTATCATGA AGGAGAAGAA GAAGCCAGGC GAGACATTCT TCATGTGCTC CTGTAGCTCC 420
GACGAGTGTA ACGATAATAT CATCTTCAGC GAGGAGTATA ACACATCCAA TCCAGACCTG 480
CTGCTGGTCA TCTTTCAGGT GACCGGAATC TCTCTGCTGC CACCACTCGG AGTGGCAATC 540
AGCGTGATCA TCATCTTCTA CTGCTATCGG GTGAACAGAC AGCAGAAGCT GTCTAGCATG 600
GGCGGCCTGG AGCCTTGTAG CAGGCTGCTG CTGCTGCCAC TGCTGCTGGC CGTGTCCGGC 660
CTGCGCCCAG TGCAGGCACA GGCACAGTCT GACTGCTCTT GTAGCACAGT GAGCCCAGGC 720
GTGCTGGCAG GAATCGTGAT GGGCGATCTG GTGCTGACCG TGCTGATCGC CCTGGCCGTG 780
TACTTTCTGG GCCGGCTGGT GCCTCGGGGC AGAGGAGCAG CAGAGGCAGC CACCAGGAAG 840
CAGCGCATCA CCGAGACAGA GAGCCCCTAC CAGGAGCTGC AGGGCCAGAG GAGCGACGTG 900
TATTCCGATC TGAACACACA GCGCCCTTAC TATAAGGGAT CTGGAGGAAG CGGAGGATCC 960
GGAGAGGGCA GAGGCTCCCT GCTGACCTGC GGCGATGTGG AGGAGAATCC AGGACCTATG 1020
CCTCCACCAA GGCTGCTGTT CTTTCTGCTG TTCCTGACAC CAATGGAGGT GCGGCCCGAG 1080
GAGCCTCTGG TGGTGAAGGT GGAGGAGGGC GACAACGCCG TGCTGCAGTG TCTGAAGGGC 1140
ACCTCTGATG GCCCCACCCA GCAGCTGACA TGGTCTAGGG AGAGCCCACT GAAGCCCTTT 1200
CTGAAGCTGA GCCTGGGCCT GCCAGGCCTG GGCATCCACA TGCGCCCTCT GGCCATCTGG 1260
CTGTTCATCT TCAACGTGAG CCAGCAGATG GGAGGCTTCT ACCTGTGCCA GCCAGGACCT 1320
CCATCTGAGA AGGCCTGGCA GCCTGGATGG ACCGTGAACG TGGAGGGAAG CGGAGAGCTG 1380
TTTCGGTGGA ACGTGAGCGA CCTGGGAGGC CTGGGATGTG GCCTGAAGAA CAGATCCTCT 1440
GAGGGCCCTA GCTCCCCATC TGGCAAGCTG ATGAGCCCAA AGCTGTACGT GTGGGCCAAG 1500
GATAGGCCAG AGATCTGGGA GGGAGAGCCA CCTTGCCTGC CACCCCGCGA CTCCCTCAAT 1560
CAGTCCCTGT CTCAGGATCT GACAATGGCC CCTGGCTCCA CCCTGTGGCT GTCTTGTGGC 1620
GTGCCTCCAG ACAGCGTGTC CAGAGGCCCA CTGTCTTGGA CCCATGTGCA CCCCAAGGGC 1680
CCTAAGTCCC TGCTGTCTCT GGAGCTGAAG GACGATCGGC CTGCCAGAGA CATGTGGGTC 1740
ATGGAGACAG GCCTGCTGCT GCCACGGGCC ACCGCACAGG ATGCCGGCAA GTACTATTGC 1800
CACAGAGGCA ACCTGACAAT GAGCTTCCAC CTGGAGATCA CCGCCCGGCC CGTGCTGTGG 1860
CACTGGCTGC TGAGAACAGG CGGCTGGAAG GTGTCTGCCG TGACCCTGGC CTACCTGATC 1920
TTCTGCCTGT GCAGCCTGGT GGGCATCCTG CACCTGCAGA GGGCCCTGGT GCTGAGGAGA 1980
AAGAGGAAGC GCATGACCGA CCCTACAAGG CGCTTT 2016
<210> SEQ ID NO 14
<211> LENGTH: 1767
<212> TYPE: DNA
<213> ORGANISM: N/A
<400> Sequence: 14
ACAATCCCCC CTCATGTGCA GAAGTCTGTG AACAATGACA TGATCGTGAC AGATAACAAT 60
GGCGCCGTGA AGTTCCCTCA GCTGTGCAAG TTCTGTGACG TGCGGTTTAG CACATGCGAT 120
AACCAGAAGT CCTGCATGTC TAATTGTAGC ATCACCTCCA TCTGCGAGAA GCCACAGGAG 160
GTGTGCGTGG CCGTGTGGAG AAAGAACGAC GAGAATATCA CCCTGGAGAC AGTGTGCCAC 240
GATCCTAAGC TGCCATACCA CGACTTTATC CTGGAGGATG CCGCCAGCCC TAAGTGTATC 300
ATGAAGGAGA AGAAGAAGCC AGGCGAGACA TTCTTCATGT GCTCCTGTAG CTCCGACGAG 360
TGTAACGATA ATATCATCTT CAGCGAGGAG TATAACACAT CCAATCCAGA CCTGCTGCTG 420
GTCATCTTTC AGCTGCGCCC AGTGCAGGCA CAGGCACAGT CTGACTGCTC TTGTAGCACA 480
GTGAGCCCAG GCGTGCTGGC AGGAATGGTG ATGGGCGATC TGGTGCTGAC CGTGCTGATC 540
GCCCTGGCCG TGTACTTTCT GGGCCGGCTG GTGCCTCGGG GCAGAGGAGC AGCAGAGGCA 600
GCCACCAGGA AGCAGCGCAT CACCGAGACA GAGAGCCCCT ACCAGGAGCT GCAGGGCCAG 660
AGGAGCGACG TGTATTCCGA TCTGAACACA CAGCGCCCTT ACTATAAGGG ATCTGAGGGC 720
AGAGGCTCCC TGCTGACCTG CGGCGATGTG GAGGAGAATC CAGGACCTAT GCCTCCACCA 780
AGGCTGCTGT TCTTTCTGCT GTTCCTGACA CCAATGGAGG TGCGGCCCGA GGAGCCTCTG 840
GTGGTGAAGG TGGAGGAGGG CGACAACGCC GTGCTGCAGT GTCTGAAGGG CACCTCTGAT 900
GGCCCCACCC AGCAGCTGAC ATGGTCTAGG GAGAGCCCAC TGAAGCCCTT TCTGAAGCTG 960
AGCCTGGGCC TGCCAGGCCT GGGTATCCAC ATGCGCCCTC TGGCCATCTG GCTGTTCATC 1020
TTCAACGTGA GCCAGCAGAT GGGAGGCTTC TACCTGTGCC AGCCAGGACC TCCATCTGAG 1080
AAGGCCTGGC AGCCTGGATG GACCGTGAAC GTGGAGGGAA GCGGAGAGCT GTTTCGGTGG 1140
AACGTGAGCG ACCTGGGAGG CCTGGGATGT GGCCTGAAGA ACAGATCCTC TGAGGGCCCT 1200
AGCTCCCCAT CTGGCAAGCT GATGAGCCCA AAGCTGTACG TGTGGGCCAA GGATAGGCCA 1260
GAGATCTGGG AGGGAGAGCC ACCTTGCCTG CCACCCCGCG ACTCCCTGAA TCAGTCCCTG 1320
TCTCAGGATC TGACAATGGC CCCTGGCTCC ACCCTGTGGC TGTCTTGTGG CGTGCCTCCA 1380
GACAGCGTGT CCAGAGGCCC ACTGTCTTGG ACCCACGTGC ACCCCAAGGG CCCTAAGTCC 1440
CTGCTGTCTC TGGAGCTGAA GGACGATCGG CCTGCCAGAG ACATGTGGGT CATGGAGACA 1500
GGCCTGCTGC TGCCACGGGC CACCGCACAG GATGCCGGGA AGTACTATTG CCACAGAGGC 1560
AACCTGACAA TGAGCTTCCA CCTGGAGATC ACCGCCCGGC CCGTGCTGTG GCACTGGCTG 1620
CTGAGAACAG GCGGCTGGAA GGTGTCTGCC GTGACCCTGG CCTACCTGAT CTTCTGCCTG 1680
TGCAGCCTGG TGGGCATCCT GCACCTGCAG AGGGCCCTGG TGCTGAGGAG AAAGAGGAAG 1740
CGCATGACCG ACCCTACAAG GCGCTTT 1767
<210> SEQ ID NO 15
<211> LENGTH: 1716
<212> TYPE: DNA
<213> ORGANISM: N/A
<400> Sequence: 15
ACAATCCCCC CTCATGTGCA GAAGTCTGTG AACAATGACA TGATCGTGAC AGATAACAA7 60
GGCGCCGTGA AGTTCCCTCA GCTGTGCAAG TTCTGTGACG TGCGGTTTAG CACATGCGAT 120
AACCAGAAGT CCTGCATGTC TAATTGTAGC ATCACCTCCA TCTGCGAGAA GCCACAGGAG 180
GTGTGCGTGG CCGTGTGGAG AAAGAACGAC GAGAATATCA CCCTGGAGAC AGTGTGCCAC 240
GATCCTAAGC TGCCATACCA CGACTTTATC CTGGAGGATG CCGCCAGCCC TAAGTGTATC 300
ATGAAGGAGA AGAAGAAGCC AGGCGAGACA TTCTTCATGT GCTCCTGTAG CTCCGACGAG 360
TGTAACGATA ATATCATCTT CAGCGAGGAG TATAACACAT CCAATCCAGA CCTGCTGCTG 420
GTCATCTTTC AGTCACCCAC TGAACCAAGC TCCAAAACCG GTAACCCCAG ACACCTGCAT 480
GTTCTGATTG GGACCTCAGT GGTCAAAATC CCTTTCACCA TCCTCCTCTT CTTTCTCCTT 540
CATCGCTGGT GCTCCAACAA AAAAAATGCT GCTGTAATGG ACCAAGAGCC TGCAGGGAAC 600
AGAACAGTGA ACAGCGAGGA TTCTGATGAA CAAGACCATC AGGAGGTGTC ATACGCAGGA 660
TCTGAGGGCA GAGGCTCCCT GCTGACCTGC GGCGATGTGG AGGAGAATCC AGGACCTATG 720
CCTCCACCAA GGCTGCTGTT CTTTCTGCTG TTCCTGACAC CAATGGAGGT GCGGCCCGAG 780
GAGCCTCTGG TGGTGAAGGT GGAGGAGGGC GACAACGCCG TGCTGCAGTG TCTGAAGGGC 840
ACCTCTGATG GCCCCACCCA GCAGCTGACA TGGTCTAGGG AGAGCCCACT GAAGCCCTTT 900
CTGAAGCTGA GCCTGGGCCT GCCAGGCCTG GGTATCCATA TGCGCCCTCT GGCGATCTGG 960
CTGTTTATCT TCAACGTGAG CCAGCAGATG GGAGGCTTCT ACCTGTGCCA GCCAGGACCT 1020
CCATCTGAGA AGGCCTGGCA GCCTGGATGG ACCGTGAACG TGGAGGGAAG CGGAGAGCTG 1080
TTTCGGTGGA ACGTGAGCGA CCTGGGAGGC CTGGGATGTG GCCTGAAGAA CAGATCCTGT 1140
GAGGGCCCTA GCTCCCCATC TGGCAAGCTG ATGAGCCCAA AGCTGTACGT GTGGGCCAAG 1200
GATAGGCCAG AGATCTGGGA GGGAGAGCCA CCTTGCCTGC CACCCCGCGA CTCCCTGAAT 1260
CAGTCCCTGT CTCAGGATCT GACAATGGCC CCTGGCTCCA CCCTGTGGCT GTCTTGTGGC 1320
GTGCCTCCAG ACAGCGTGTC CAGAGGCCCA CTGTCTTGGA CCCACGTGCA CCCCAAGGGC 1380
CCTAAGTCCC TGCTGTCTCT GGAGCTGAAG GACGATCGGC CTGCCAGAGA CATGTGGGTC 1440
ATGGAGACAG GCCTGCTGCT GCCACGGGCC ACCGCACAGG ATGCCGGCAA GTACTATTGC 1500
CACAGAGGCA ACCTGACAAT GAGCTTCCAC CTGGAGATCA CCGCCCGGCC CGTGCTGTGG 1560
CACTGGCTGC TGAGAACAGG CGGCTGGAAG GTGTCTGCCG TGACCCTGGC CTACCTGATC 1620
TTCTGCCTGT GCAGCCTGGT GGGCATCCTG CACCTGCAGA GGGCCCTGGT GCTGAGGAGA 1680
AAGAGGAAGC GCATGACCGA CCCTACAAGG CGCTTT 1716
<210> SEQ ID NO 16
<211> LENGTH: 8179
<212> TYPE: DNA
<213> ORGANISM: N/A
<400> Sequence: 16
AAGCTTTGCT CTTAGGAGTT TCCTAATACA TCCCAAACTC AAATATATAA AGCATTTGAC 60
TTGTTCTATG CCCTAGGGGG CGGGGGGAAG CTAAGCCAGC TTTTTTTAAC ATTTAAAATG 120
TTAATTCCAT TTTAAATGCA CAGATGTTTT TATTTCATAA GGGTTTCAAT GTGCATGAAT 180
GCTGCAATAT TCCTGTTACC AAAGCTAGTA TAAATAAAAA TAGATAAACG TGGAAATTAC 240
TTAGAGTTTC TGTCATTAAC GTTTCCTTCC TCAGTTGACA ACATAAATGC GCTGCTGAGC 300
AAGCCAGTTT GCATCTGTCA GGATCAATTT CCCATTATGC CAGTCATATT AATTACTAGT 360
CAATTAGTTG ATTTTTATTT TTGACATATA CATGTGAATG AAAGACCCCA CCTGTAGGTT 420
TGGCAAGCTA GCTTAAGTAA CGCCATTTTG CAAGGCATGG AAAAATACAT AACTGAGAAT 480
AGAAAAGTTC AGATCAAGGT CAGGAACAGA TGGAACAGCT GAATATGGGC CAAACAGGAT 540
ATCTGTGGTA AGCAGTTCCT GCCCCGGCTC AGGGCCAAGA ACAGATGGAA CAGCTGAATA 600
TGGGCCAAAC AGGATATCTG TGGTAAGCAG TTCCTGCCCC GGCTCAGGGC CAAGAACAGA 660
TGGTCCCCAG ATGCGGTCCA GCCCTCAGCA GTTTCTAGAG AACCATCAGA TGTTTCCAGG 720
GTGCCCCAAG GACCTGAAAT GACCCTGTGC CTTATTTGAA CTAACCAATC AGTTCGCTTC 780
TCGCTTCTGT TCGCGCGCTT ATGCTCCCCG AGCTCAATAA AAGAGCCCAC AACCCCTCAC 840
TCGGGGCGCC AGTCCTCCGA TTGACTGAGT CGCCCGGGTA CCCGTGTATC CAATAAACCC 900
TCTTGCAGTT GCATCCGACT TGTGGTCTCG CTGTTCCTTG GGAGGGTCTC CTCTGAGTGA 960
TTGACTACCC GTCAGCGGGG GTCTTTCATT TGGGGGCTCG TCCGGGATCG GGAGACCCCT 1020
GCCCAGGGAC CACCGACCCA CCACCGGGAG GTAAGCTGGC CAGCAACTTA TCTGTGTCTG 1080
TCCGATTGTC TAGTGTCTAT GACTGATTTT ATGCGCCTGC GTCGGTACTA GTTAGCTAAC 1140
TAGCTCTGTA TCTGGCGGAC CCGTGGTGGA ACTGACGAGT TCGGAACACC CGGCCGCAAC 1200
CCTGGGAGAC GTCCCAGGGA CTTCGGGGGC CGTTTTTGTG GCCCGACCTG AGTCCTAAAA 1260
TCCCGATCGT TTAGGACTCT TTGGTGCACC CCCCTTAGAG GAGGGATATG TGGTTCTGGT 1320
AGGAGACGAG AACCTAAAAC AGTTCCCGCC TCCGTCTGAA TTTTTGCTTT CGGTTTGGGA 1380
CCGAAGCCGC GCCGCGCGTC TTGTCTGCTG CAGCATCGTT CTGTGTTGTC TCTGTCTGAC 1440
TGTGTTTCTG TATTTGTCTG AAAATATGGG CCCGGGCTAG CCTGTTACCA CTCCCTTAAG 1500
TTTGACCTTA GGTCACTGGA AAGATGTCGA GCGGATCGCT CACAACCAGT CGGTAGATGT 1560
CAAGAAGAGA CGTTGGGTTA CCTTCTGCTC TGCAGAATGG CCAACCTTTA ACGTCGGATG 1620
GCCGCGAGAC GGCACCTTTA ACCGAGACCT CATCACCCAG GTTAAGATCA AGGTCTTTTC 1680
ACCTGGCCCG CATGGACACC CAGACCAGGT GGGGTACATC GTGACCTGGG AAGCCTTGGC 1740
TTTTGACCCC CCTCCCTGGG TCAAGCCCTT TGTACACCCT AAGCCTCCGC CTCCTCTTCC 1800
TCCATCCGCC CCGTCTCTCC CCCTTGAACC TCCTCGTTCG ACCCCGCCTC GATCCTCCCT 1860
TTATCCAGCC CTCACTCCTT CTCTAGGCGC CCCCATATGG CCATATGAGA TCTTATATGG 1920
GGCACCCCCG CCCCTTGTAA ACTTCCCTGA CCCTGACATG ACAAGAGTTA CTAACAGCCC 1980
CTCTCTCCAA GCTCACTTAC AGGCTCTCTA CTTAGTCCAG CACGAAGTCT GGAGACCTCT 2040
GGCGGCAGCC TACCAAGAAC AACTGGACCG ACCGGTGGTA CCTCACCCTT ACCGAGTCGG 2100
CGACACAGTG TGGGTCCGCC GACACCAGAC TAAGAACCTA GAACCTCGCT GGAAAGGACC 2160
TTACACAGTC CTGCTGACCA CCCCCACCGC CCTCAAAGTA GACGGCATCG CAGCTTGGAT 2220
ACACGCCGCC CACGTGAAGG CTGCCGACCC CGGGGGTGGA CCATCCTCTA GACTGCCATG 2280
GGAAGGGGCC TGCTGAGAGG CCTGTGGCCC CTGCACATCG TGCTGTGGAC CAGGATCGCC 2340
TCCACAATCC CCCCTCATGT GCAGAAGTCT GTGAACAATG ACATGATCGT GACAGATAAC 2400
AATGGCGCCG TGAAGTTCCC TCAGCTGTGC AAGTTCTGTG ACGTGCGGTT TAGCACATGC 2460
GATAACCAGA AGTCCTGCAT GTCTAATTGT AGCATCACCT CCATCTGCGA GAAGCCACAG 2520
GAGGTGTGCG TGGCCGTGTG GAGAAAGAAC GACGAGAATA TCACCCTGGA GACAGTGTGC 2580
CACGATCCTA AGCTGCCATA CCACGACTTT ATCCTGGAGG ATGCCGCCAG CCCTAAGTGT 2640
ATCATGAAGG AGAAGAAGAA GCCAGGCGAG ACATTCTTCA TGTGCTCCTG TAGCTCCGAC 2700
GAGTGTAACG ATAATATCAT CTTCAGCGAG GAGTATAACA CATCCAATCC AGACCTGCTG 2760
CTGGTCATCT TTCAGCTGCG CCCAGTGCAG GCACAGGCAC AGTCTGACTG CTCTTGTAGC 2820
ACAGTGAGCC CAGGCGTGCT GGCAGGAATC GTGATGGGCG ATCTGGTGCT GACCGTGCTG 2880
ATCGCCCTGG CCGTGTACTT TCTGGGCCGG CTGGTGCCTC GGGGCAGAGG AGCAGCAGAG 2940
GCAGCCACCA GGAAGCAGCG CATCACCGAG ACAGAGAGCC CCTACCAGGA GCTGCAGGGC 3000
CAGAGGAGCG ACGTGTATTC CGATCTGAAC ACACAGCGCC CTTACTATAA GGGATCTGAG 3060
GGCAGAGGCT CCCTGCTGAC CTGCGGCGAT GTGGAGGAGA ATCCAGGACC TATGCCTCCA 3120
CCAAGGCTGC TGTTCTTTCT GCTGTTCCTG ACACCAATGG AGGTGCGGCC CGAGGAGCCT 3180
CTGGTGGTGA AGGTGGAGGA GGGCGACAAC GCCGTGCTGC AGTGTCTGAA GGGCACCTCT 3240
GATGGCCCCA CCCAGCAGCT GACATGGTCT AGGGAGAGCC CACTGAAGCC CTTTCTGAAG 3300
CTGAGCCTGG GCCTGCCAGG CCTGGGTATC CACATGCGCC CTCTGGCCAT CTGGCTGTTC 3360
ATCTTCAACG TGAGCCAGCA GATGGGAGGC TTCTACCTGT GCCAGCCAGG ACCTCCATCT 3420
GAGAAGGCCT GGCAGCCTGG ATGGACCGTG AACGTGGAGG GAAGCGGAGA GCTGTTTCGG 3480
TGGAACGTGA GCGACCTGGG AGGCCTGGGA TGTGGCCTGA AGAACAGATC CTCTGAGGGC 3540
CCTAGCTCCC CATCTGGCAA GCTGATGAGC CCAAAGCTGT ACGTGTGGGC CAAGGATAGG 3600
CCAGAGATCT GGGAGGGAGA GCCACCTTGC CTGCCACCCC GCGACTCCCT GAATCAGTCC 3660
CTGTCTCAGG ATCTGACAAT GGCCCCTGGC TCCACCCTGT GGCTGTCTTG TGGCGTGCCT 3720
CCAGACAGCG TGTCCAGAGG CCCACTGTCT TGGACCCACG TGCACCCCAA GGGCCCTAAG 3780
TCCCTGCTGT CTCTGGAGCT GAAGGACGAT CGGCCTGCCA GAGACATGTG GGTCATGGAG 3840
ACAGGCCTGC TGCTGCCACG GGCCACCGCA CAGGATGCCG GCAAGTACTA TTGCCACAGA 3900
GGCAACCTGA CAATGAGCTT CCACCTGGAG ATCACCGCCC GGCCCGTGCT GTGGCACTGG 3960
CTGCTGAGAA CAGGCGGCTG GAAGGTGTCT GCCGTGACCC TGGCCTACCT GATCTTCTGC 4020
CTGTGCAGCC TGGTGGGCAT CCTGCACCTG CAGAGGGCCC TGGTGCTGAG GAGAAAGAGG 4080
AAGCGCATGA CCGACCCTAC AAGGCGCTTT TAAGGATCCG GATTAGTCCA ATTTGTTAAA 4140
GACAGGATAT CAGTGGTCCA GGCTCTAGTT TTGACTCAAC AATATCACCA GCTGAAGCCT 4200
ATAGAGTACG AGCCATAGAT AAAATAAAAG ATTTTATTTA GTCTCCAGAA AAAGGGGGGA 4260
ATGAAAGACC CCACCTGTAG GTTTGGCAAG CTAGCTTAAG TAACGCCATT TTGCAAGGCA 4320
TGGAAAAATA CATAACTGAG AATAGAGAAG TTCAGATCAA GGTCAGGAAC AGATGGAACA 4380
GCTGAATATG GGCCAAACAG GATATCTGTG GTAAGCAGTT CCTGCCCCGG CTCAGGGCCA 4440
AGAACAGATG GAACAGCTGA ATATGGGCCA AACAGGATAT CTGTGGTAAG CAGTTCCTGC 4500
CCCGGCTCAG GGCCAAGAAC AGATGGTCCC CAGATGCGGT CCASCCCTCA GCAGTTTCTA 4560
CAGAACCATC AGATGTTTCC AGGGTGCCCC AAGGACCTGA AATGACCCTG TGCCTTATTT 4620
GAACTAACCA ATCAGTTCGC TTCTCGCTTC TGTTCGCGCG CTTCTGCTCC CCGAGCTCAA 4680
TAAAAGAGCC CACAACCCCT CACTCGGGGC GCCAGTCCTC CGATTGACTG AGTCGCCCGG 4740
GTACCCGTGT ATCCAATAAA CCCTCTTGCA GTTGCATCCG ACTTGTGGTC TCGCTGTTCC 4800
TTGGGAGGGT CTCCTCTGAG TGATTGACTA CCCGTCAGCG GGGGTCTTTC ACACATGCAG 4860
CATGTATCAA AATTAATTTG GTTTTTTTTC TTAAGTATTT ACATTAAATG GCCATAGTAC 4920
TTAAAGTTAC ATTGGCTTCC TTGAAATAAA CATGGAGTAT TCAGAATGTG TCATAAATAT 4980
TTCTAATTTT AAGATAGTAT CTCCATTGGC TTTCTACTTT TTCTTTTATT TTTTTTTGTC 5040
CTCTGTCTTC CATTTGTTGT TGTTGTTGTT TGTTTGTTTG TTTGTTGGTT GGTTGGTTAA 5100
TTTTTTTTTA AAGATCCTAC ACTATAGTTC AAGCTAGACT ATTAGCTACT CTGTAACCCA 5160
GGGTGACCTT GAAGTCATGG GTAGCCTGCT GTTTTAGCCT TCCCACATCT AAGATTACAG 5220
GTATGAGCTA TCATTTTTGG TATATTGATT GATTGATTGA TTGATGTGTG TGTGTGTGAT 5280
TGTGTTTGTG TGTGTGACTG TGAAAATGTG TGTATGGGTG TGTGTGAATG TGTGTATGTA 5340
TGTGTGTGTG TGAGTGTGTG TGTGTGTGTG TGCATGTGTG TGTGTGTGAC TGTGTCTATG 5400
TGTATGACTG TGTGTGTGTG TGTGTGTGTG TGTGTGTGTG TGTGTGTGTG TGTGTTGTGA 5460
AAAAATATTC TATGGTAGTG AGAGCCAACG CTCCGGCTCA GGTGTCAGGT TGGTTTTTGA 5520
GAGAGAGTCT TTCACTTAGC TTGGAATTCA CTGGCCGTCG TTTTACAACG TCGTGACTGG 5580
GAAAACCCTG GCGTTACCCA ACTTAATCGC CTTGCAGCAC ATCCCCCTTT CGCCAGCTGG 5640
CGTAATAGCG AAGAGGCCCG CACCGATCGC CCTTCCCAAC AGTTGCGCAG CCTGAATGGC 5700
GAATGGCGCC TGATGCGGTA TTTTCTCCTT ACGCATCTGT GCGGTATTTC ACACCGCATA 5760
TGGTGCACTC TUAGTACAAT CTGCTCTGAT GCCGCATAGT TAAGCCAGCC CCGACACCCG 5820
CCAACACCCG CTGACGCGCC CTGACGGGCT TGTCTGCTCC CGGCATCCGC TTACAGACAA 5880
GCTGTGACCG TCTCCGGGAG CTGCATGTGT CAGAGGTTTT CACCGTCATC ACCGAAACGC 5940
GCGATGACGA AAGGGCCTCG TGATACGCCT ATTTTTATAG GTTAATGTCA TGATAATAAT 6000
GGTTTCTTAG ACGTGAGGTG GCACTTTTCG GGGAAATGTG CGCGGAACCC CTATTTGTTT 6060
ATTTTTCTAA ATACATTCAA ATATGTATCC GCTCATGAGA CAATAACCCT GATAAATGCT 6120
TCAATAATAT TGAAAAAGGA AGAGTATGAG TATTCAACAT TTCCGTGTCG CCCTTATTCC 6180
CTTTTTTGCG GCATTTTGCC TTCCTGTTTT TGCTCACCCA GAAACGCTGG TGAAAGTAAA 6240
AGATGCTGAA GATCAGTTGG GTGCACGAGT GGGTTACATC GAACTGGATC TCAACAGCGG 6300
TAAGATCCTT GAGAGTTTTC GCCCCGAAGA ACGTTTTCCA ATGATGAGCA CTTTTAAAGT 6360
TCTGCTATGT GGCGCGGTAT TATCCCGTAT TGACGCCGGG CAAGAGCAAC TCGGTCGCCG 6420
CATAGACTAT TCTCAGAATG ACTTGGTTGA GTACTCACCA GTCACAGAAA AGCATCTTAC 6480
GGATGGCATG ACAGTAAGAG AATTATGCAG TGCTGCCATA ACOATGAGTG ATAACACTGC 6540
GGCCAACTTA CTTCTGACAA CGATCGGAGG ACCGAAGGAG CTAACCGCTT TTTTGCACAA 6600
CATGGGGGAT CATGTAACTC GCCTTGATCG TTGGGAACCG GAGCTGAATG AAGCCATACC 6660
AAACGACGAG CGTGACACCA CGATGCCTGT AGCAATGGCA ACAACGTTGC GCAAACTATT 6720
AACTGGCGAA CTACTTACTC TAGCTTCCCG GCAACAATTA ATAGACTGGA TGGAGGCGGA 6780
TAAAGTTGCA GGACCACTTC TGCGCTCGGC CCTTCCGGCT GGCTGGTTTA TTGCTGATAA 6840
ATCTGGAGCC GGTGAGCGTG GGTCTCGCGG TATCATTGCA GCACTGGGGC CAGATGGTAA 6900
GCCCTCCCGT ATCGTAGTTA TCTACACGAC GGGGAGTCAG GCAACTATGG ATGAACGAAA 6960
TAGACAGATC GCTGAGATAG GTGCCTCACT GATTAAGCAT TGGTAACTGT CAGACCAAGT 7020
TTACTCATAT ATACTTTAGA TTGATTTAAA ACTTCATTTT TAATTTAAAA GGATCTAGGT 7080
GAAGATCCTT TTTGATAATC TCATGACCAA AATCCCTTAA CGTGAGTTTT CGTTCCACTG 7140
AGCGTCAGAC CCCGTAGAAA AGATCAAAGG ATCTTCTTGA GATCCTTTTT TTCTGCGCGT 7200
AATCTGCTGC TTGCAAACAA AAAAACCACC GCTACCAGCG GTGGTTTGTT TGCCGGATCA 7260
AGAGCTACCA ACTCTTTTTC CGAAGGTAAC TGGCTTCAGC AGAGCGCAGA TACCAAATAC 7320
TGTCCTTCTA GTGTAGCCGT AGTTAGGCCA CCACTTCAAG AACTCTGTAG CACCGCCTAC 7380
ATACCTCGCT CTGCTAATCC TGTTAGCAGT GGCTGCTGCC AGTGGCGATA AGTCGTGTCT 7440
TACCGGGTTG GACTCAAGAC GATAGTTACC GGATAAGGCG CAGCGGTCGG GCTGAACGGG 7500
GGGTTCGTGC ACACAGCCCA GCTTGGAGCG AACGAGCTAC ACCGAACTGA GATACCTACA 7560
GCGTGAGCAT TGAGAAAGCG CCACGCTTCC CGAAGGGAGA AAGGCGGACA GGTATCCGGT 7620
AAGCGGCAGG GTCGGAACAG GAGAGCGCAC GAGGGAGCTT CCAGGGGGAA ACGCCTGGTA 7680
TCTTTATAGT CCTGTCGGGT TTCGCCACCT CTGACTTGAG CGTCGATTTT TGTGATGCTC 7740
GTCAGGGGGG CGGAGCCTAT GGAAAAACGC CAGCAACGCG CzCCTTTTAC GGTTCCTGGC 7800
CTTTTGCTGG CCTTTTGCTC ACATGTTCTT TCCTGCGTTA TCCCCTGATT CTGTGGATAA 7860
CCGTATTACC GCCTTTGAGT GAGCTGATAC CGCTCGCCGC AGCCGAACGA CCGAGCGCAG 7920
CGAGTCAGTG AGCGAGGAAG CGGAAGAGCG CCCAATACGC AAACCGCCTC TCCCCGCGCG 7980
TTGGCCGATT CATTAATGCA GCTGGCACGA CAGGTTTCCC GACTGGAAAG CGGGCAGTGA 8040
GCGCAACGCA ATTAATGTGA GTTAGCTCAC TCATTAGGCA CCCCAGGCTT TACACTTTAT 8100
GCTTCCGGCT CGTATGTTGT GTGGAATTGT GAGCGGATAA CAATTTCACA CAGGAAACAG 8160
CTATGACCAT GATTACGCC 8179

### Materials and Methods

### Cell Sources and Cell Lines

Umbilical cord blood mononuclear cells were harvested from fresh cord blood units obtained from MD Anderson Cancer Center (Houston. TX) under approved Institutional review board-approved protocols (Pro00003896) by density gradient separation, and NK cells were isolated by negative selection with the EasySep Human NK Cell Isolation Kit (StemCell Technologies). Cord blood units were obtained under informed written consent and in accordance to the Declaration of Helsinki and the guidelines of the Institutional Review Board at MDACC (Houston, TX). After 24 hours of activation with 10 ng/mL of human IL15 (R&D Systems), NK cells were stimulated with K562 feeder cells, modified to express membrane-bound IL15 and 41BBL (refs. 31, 37; generously obtained from Baylor College of Medicine, Houston, TX; Pro00003869), irradiated at 200 Gy and cultured with NK cells at a 2:1 K562:NK-cell ratio. NK cells were expanded in Stem Cell Growth Medium (CellGenix) supplemented with 200 IU/mL human IL2, 15 ng/mL human IL15, 10% FBS (Gibco, Thermo Fisher Scientific), and 1% Glutamax (Gibco. Thermo Fisher Scientific). NK cells were isolated from 30 total cord blood donors for downstream use, and untransduced and transduced cells were generated from each individual donor line. Sample size (number of donor-derived lines) used for each experiment is specified in each figure legend. Modified and unmodified K562 cell lines were cultured with Iscove’s modified Dulbecco’s medium (Thermo Fisher Scientific) supplemented with 10% FBS (Gibco, Thermo Fisher Scientific), 1% penicillin-streptomycin, and 1% Glutamax (Gibco, Thermo Fisher Scientific) Neuroblastoma line SHSY5Y was purchased from ATCC and grown in a 1:1 medium of DMEM and F12K medium supplemented with 10% FBS (Gibco, Thermo Fisher Scientific), and 1% Glutamax (Gibco, Thermo Fisher Scientific). We performed HLA and short tandem repeat profiling to verify the identity and type of the SHSY5Y tumor line (Genetica Cell Line Testing). We also verified that the SFISY5Y neuroblastoma line produces high levels of TGFβ in vivo from SHSY5Y-inoculated NSG mice, and expresses low levels of MHC class I molecules (Supplementary FIG. S1). For generating the bioluminescent neuroblastoma line used in vivo, SHSY5Y was transduced with 2.5 × 10⁶ CFU of CMV-Firefly-luciferase-puro-resistant (Cellomics Technology) as per manufacturer’s protocol. Bioluminescence was assessed with the Pierce Luciferase Dual Assay Kit (Thermo Fisher Scientific) and positive clones isolated by puromycin resistance and expanded for use, and the cell line was identified as SHSY5Y-luc. Identical in vitro experiments were performed with the neuroblastoma line HTLA230, purchased from ATCC.

### Generation of Plasmids and Retrovirus Production

Three modified plasmids were constructed as follows (FIG. 2A): (I) RBDNR: human type II TGFβ receptor cDNA was truncated at nt597 as described previously (38) and coupled to a truncated CD19 tag and pac puromycin-resistant gene via T2A sequences. (ii) NKA: human type II TGFβ receptor cDNA was truncated at nt597 as described previously (38), containing extracellular and transmembrane moieties, and coupled to the transmembrane and intracellular coding region of DAP12 as derived from full-length DAP12 cDNA (39), a truncated CD19 tag and a pac puromycin-resistant gene via T2A sequences. (iii) NKCT: human type II TGFβ receptor cDNA was truncated at nt597 as described previously (38) and coupled to a “SynNotch” receptor (26) composed of the Notch 1 minimal regulatory region fused to the DNA binding domain for RELA (p65) and a VP64 effector domain (40), coupled to a truncated CD19 tag and a pac puromycin-resistant gene via T2A sequences. The RBDNR, NKA, and NKCT constructs were then individually integrated at the BaniHI and NcoI sites of the retroviral vector SFG to generate plasmids of the same name. A control GFP-containing plasmid was generated elsewhere (41). Phoenix-ecotropic cells (ATCC) were transfected with SFGRBDNR, SFG:NKA, and SFGNKCT, with Lipofectamine 2000 (Thermo Fisher Scientific) reagents used as per manufacturer’s protocol . Transient retroviral supernatant was collected 48 and 72 hours after transfection and was used to transduce the PG13-stable packaging cell line (ATCC). Transduced PG13 cells were evaluated for transduction efficiency as described below, and single-cell FACS sorting was performed to isolate single clonally-derived producer lines for RBDNR, NKA, and NKCT constructs. For FACS sorting, single cells that expressed high levels of CD19 and TGFβRII expression were isolated with the Becton Dickinson Influx Cell Sorter (BD Biosciences) and selectively expanded in puromycin-containing DMEM with 10% FBS (Gibco, Thermo Fisher Scientific) and 1% Glutamax (Gibco, Thermo Fisher Scientific). Retroviral supernatants containing RBDNR, NKA, NKC and NKA2 constructs were harvested from subconfluent PG 13 cells, passed through a 0.45-µm filter, and stored at -80◦C until needed for transduction .

### NK-Cell Transduction and Expansion

Activated NK cells were plated on retronectin-coated nontissue culture---treated plates (Takara) and transduced with RBDNR, NKA, or NKCT-containing retroviral supernatant in the presence of IL2 (200 IU/mL). After transductions, NK cells were assessed for transduction efficiency by staining with antibodies against CD19 conjugated to allophycocyanin (BD Biosciences) and TGFβRII conjugated to phycoerythrin (R&D Systems). After transduction, NK cells were expanded with additional stimulations with irradiated modified K562s, as described above, and exogenous IL2 and IL15. To enrich for phenotypic, functional, and in vivo assays, transduced NK cells were stained with CD19 microbeads (Miltenyi Biotec), and enriched by positive immunomagnetic bead selection according to the manufacturer’s protocol.

### Phenotypic and Functional Assessment of NK Cells

NK cells were harvested from 21- or 28-day cultures, washed with FACS buffer, and incubated with human FcR Blocking Reagent for 10 minutes (Miltenyi Biotec). 21-day cultures were used for analysis of NK-cell molecular signaling, whereas 28-day cultures were used for all other endpoint NK-cell assays including phenotype, cytotoxicity, and in vivo applications, to allow for maximal cell expansion. Unmodified and modified NK cells, or cell lines, were stained with antibodies specific for NKp30, NKG2D, NKp44, CD16, PD1, CD56, CD3, DNAM1, CD19, TGFβRII (R&D Systems), HLA-ABC, or MICA/B. Antibodies were conjugated to FITC, PE, PerCP, APC, APC-Cy7, Pe-Cy7, or PerCP-Cy5.5 (BD Biosciences, unless otherwise identified). Samples were run on the Accuri C6 (BD Biosciences) or CytoFLEX S (Beckman Coulter) flow cytometers and analysis conducted using Flow Jo 7.6.5 (FlowJo LLC). To assess the cytokine profile of transduced and untransduced NK cells, cell supernatant was harvested from 21/28-day NK cultures and used in the Bio-Plex Human Cytokine 17-plex Assay according to the manufacturer’s instructions (Bio-Rad Laboratories). For examination of cellular proliferation at endpoint, NK cells were labeled with carboxyfluorescein succinimidyl ester (CFSE) as per manufacturer’s protocol (Thermo Fisher Scientific) and cocultured with modified K562 cells for 72 hours after assay establishment. To determine the cytolytic properties of unmodified and modified NK cells in various conditions, standard ⁵¹Cr release cytotoxicity assays were performed as described elsewhere (22) NK cells were incubated with ⁵¹Cr-labeled target cells (unmodified K562s, SHSY5Y cell lines-loaded with 10 µCi ⁵¹Cr per 10,000 cells) at 40: 1, 20:1, 10:1, and 5:1 ratios for 5 hours in triplicate, and percent killing was determined by the following formula: (experimental count ... spontaneous count)/(maximum count ... spontaneous count) x 100%. For phenotypic and functional assessment of NK cells after exposure to TGFβ, NK cells were cultured with 10 ng/ml, TGFβ (activated with 4 mmol/L HCl) added every other day. Five days after assay establishment, NK cells were isolated and examined by flow cytometry, multiplex assays, or cytotoxicity assays, as described above. Further details of NK-cell culture can be found in the Supplementary Data.

### Molecular Assessment of NK Cells After TGFβ Exposure

To examine the molecular effects of TGFβ unmodified and modified NK cells (from 21-day cultures, were cultured with 10 ng/mL TGFβ (activated with 4 mmol/L HCI) at 37° C. At 30 minutes, 1, 3, 24, 48, and 72 hours post-TGFβ addition protein was isolated for molecular assessment. Briefly, unmodified or modified NK cells were pelleted and resuspended in RIPA lysis buffer (Thermo Fisher Scientific) containing protease inhibitor and phosphatase inhibitor cocktails (Roche Diagnostics) . After 10-minute incubation at 4° C., protein was isolated and particulate matter removed by filtration with Ultrafree-CL centrifugal filter units (EMD Millipore). Protein was quantified with a Pierce BCA Protein Assay Kit (Thermo Fisher Scientific) and 30 µg of protein lysate was isolated and used in the TGFβ Signaling Pathway Magnetic Bead 6-plex Cell Signaling Multiplex Assay (EMD Millipore) as per manufacturer’s instructions. Protein expression of phospho-Akt (Ser473), phospho-ERK (Thrt85/Tyr187), phospho-Smad2 (Ser465/467), and phospho-Smad3 (Ser423/425) was quantitated with Luminex xMap detection, based on positive and negative quantified protein controls.

### Mice and in Vivo Experiments

Male and female NSG (NOD.Cg-Prkdc^{seid}112rg^{tm(Wj)}/SzJ mice were purchased from Jackson Laboratories and bred in-house in accordance with approved protocols with the Institutional Animal Care and Use Committee at Children’s National Health System (Washington, D.C.). For in vivo neuroblastoma treatment experiments, 6- to 10-week-old male and female mice were preconditioned with sublethal irradiation (300 cGy) and inoculated with 2.5 × 10⁶ SHSY5Y-luc cells, administered subcutaneously in the dorsal flank of animals . This sublethal irradiation was performed at doses similar to that reported by other groups, which has verified successful immune depletion and immune engraftment in these models (42-45).

Animals were treated immediately following inoculation, a model commonly used in the field (43), with systemic administration of 1.5 × 10⁷ unmodified or modified NK cells via tail veins. For long-term studies, animals received weekly doses of 5-10 × 10⁶ unmodified or modified NK cells, administered systemically (5 doses in total). All mice were treated with 0.2 µg human IL2, administered intraperitoneally every other day over the course of their cell therapy doses. The SHSY5Y neuroblastoma line was specifically chosen over the HTLA230 neuroblastoma line due its superior production of TGFβ both in vitro and in vivo in preliminary xenograft experiments. In addition, The SHSY5Y neuroblastoma line derives from the SK-N-SH line originating from a 4-year-old neuroblastoma patient and is a well-established neuroblastoma line used in the field and published in other immunotherapy studies (46-50). For examination of tumor progression, animals were imaged every other day with the IVIS Lumina 100 (PerkinElmer), and images were scaled to the same minimum and maximum photon distribution prior to analysis Animals were injected with 150 mg/kg Xeno-Light D~Luciferin (PerkinElmer) 10 minutes prior to imaging with the IVIS, during which time animals were anesthetized with 2% isoflurane. Bioluminescent images were captured with 15-second exposure, with small binning and f-stop 2, and total bioluminescence was quantified by photon counts under individual murine regions of interest (photon counts) . For analysis of NK-cell persistence, blood was collected at designated time points from submandibular veins with Goldenrod Animal Lancets (Braintree Scientific Inc.) and stored in K2EDTA-containing Microtainer tubes (BD Biosciences) at -80° C.

### Assessment of NK-Cell Persistence in Vivo

Transduced NK cells were detected and quantified in the peripheral blood using digital droplet PCR (ddPCR) methods. RNA was extracted from collected blood using the Whole Blood Quick-RNA Kit according to the manufacturer’s instructions (Zymo Research). cDNA was prepared from 2,000 ng of isolated RNA by performing PCR amplification with RT buffer, dNTP Mix, MultiScribe RT, RNAse inhibitor, random primers, and nuclease-free water according to the High Capacity RT cDNA Kit (Thermo Fisher Scientific) and samples were run with the BioRad QC200 Droplet system according to manufacturer’s protocols (Bio-Rad Laboratories Inc.). For identification of NK cells, primers specific to GFP, RBDNR, NKA, and NKCT construct were used, as described in the Supplementary Data and Methods.

### Statistical Analysis

All experiments were performed in duplicate or triplicate, with sample sizes indicated in each corresponding figure legend. Data were analyzed using GraphPad Prism software (GraphPad), and across all figures the solid color bars indicate non-TGFβ-treated groups, whereas striped bars indicate TGFβ-treated groups. Comparisons between untransduced, RBDNR, NKA, and NKCT data were performed using Student l test or X² tests, with P < 0.05 considered as significant and denoted with an asterisk (*) and P < 0.0001 denoted with a two asterisks (**), unless otherwise noted . For in vivo experiments, we performed the log-rank (Mantel-Cox) test for Kaplan--Meier---generated survival data, with P < 0.05 considered as significant. Schematic signaling diagrams were generated using Biorender.

### Results

### Variant TGFβ Receptor-Modified NK Cells Are Phenotypically and Functionally Similar To Unmodified NK Cells

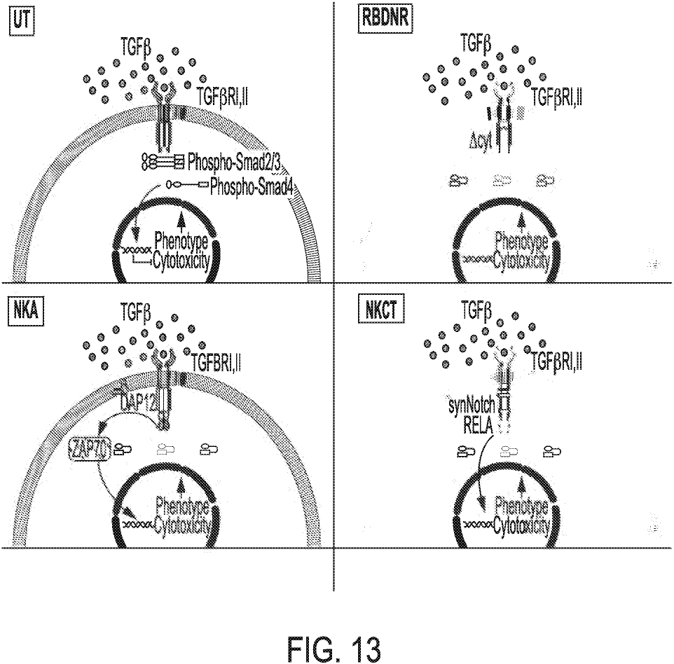

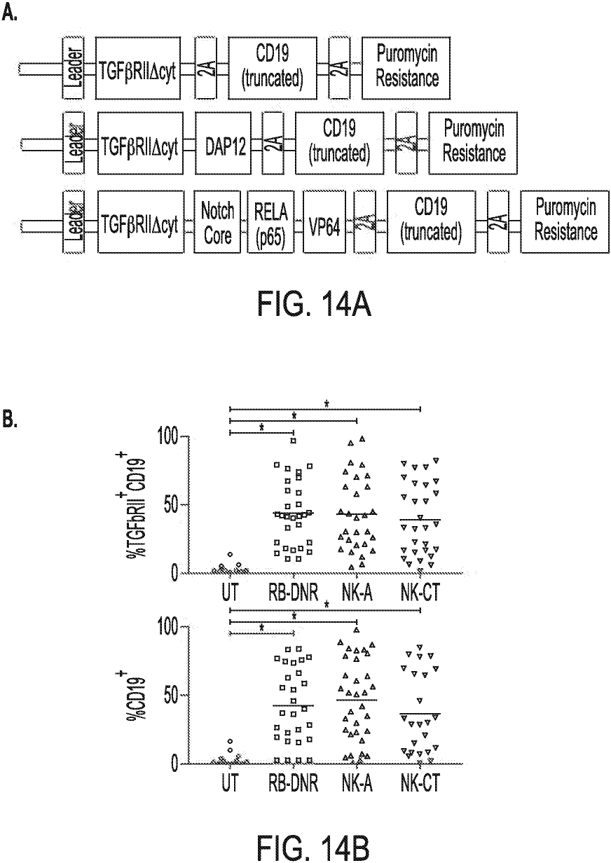

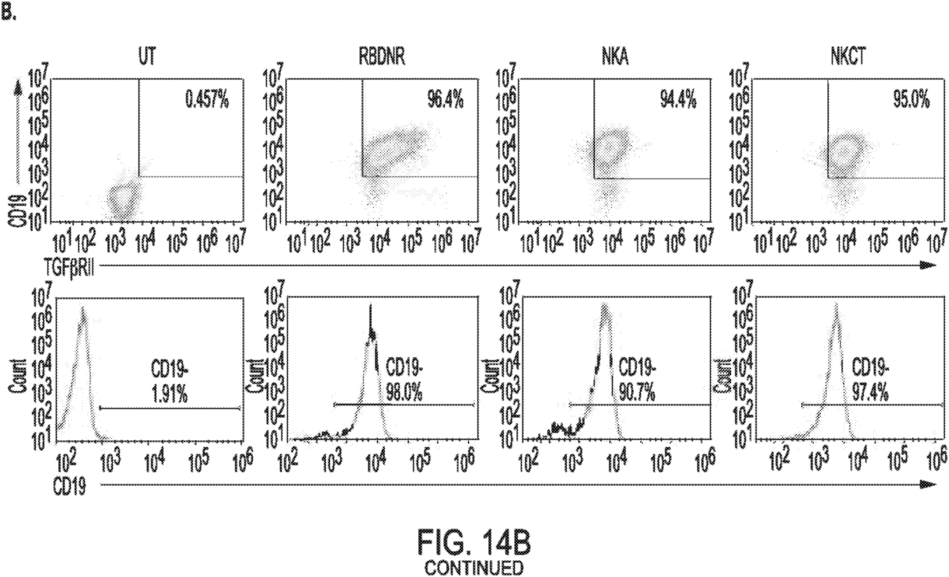

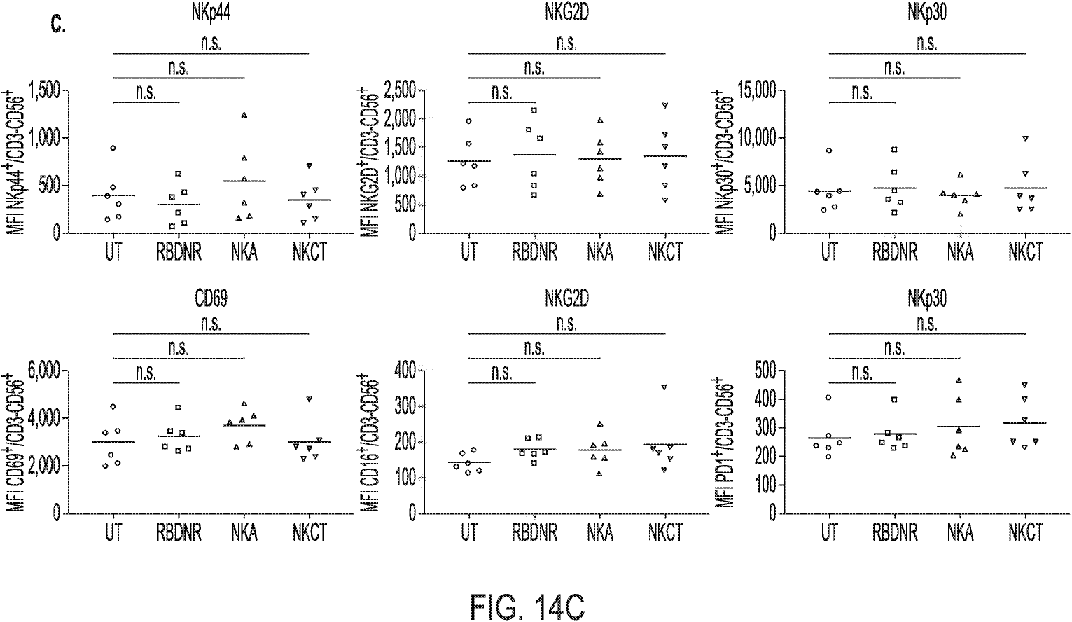

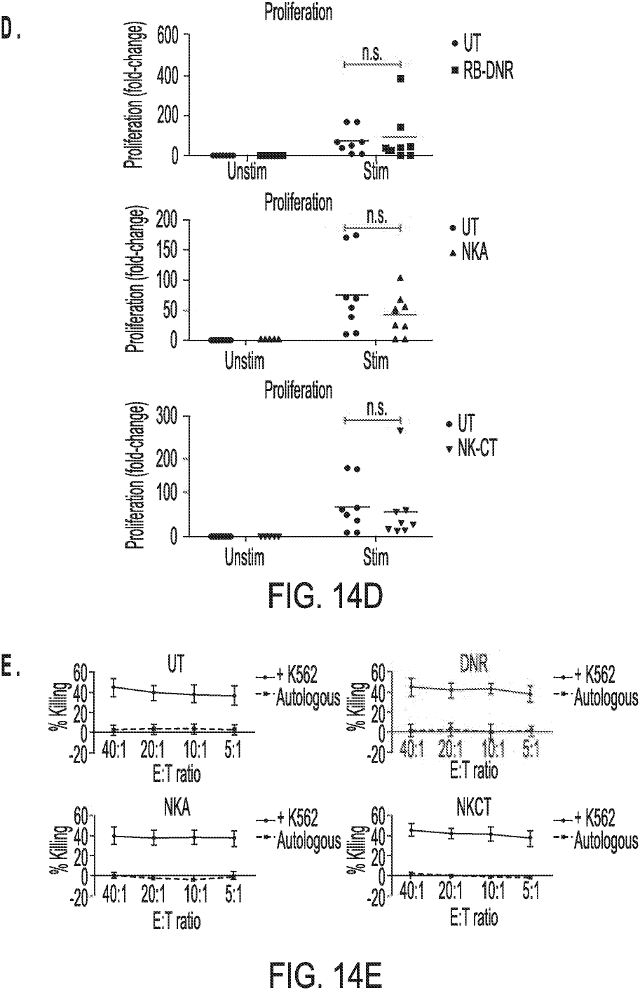

To examine NK-cell phenotype and function following genetic modification of the TGFβ receptor, cord blood-derived NK cells (33, 34, 36) were isolated and stimulated with irradiated feeder cells and supplemented with recombinant human IL.2 and IL15 (31, 37). Four days after stimulation, NK cells were divided in to four groups: untransduced (UT), RBDNR-transduced, NKA-transduced, and NKCT-transduced NK cells as described (FIG. 14A). Cord blood-derived NK cells were successfully transduced with RBDNR, NKA, or NKCT variant TGFβ receptors, as indicated by surface staining of TGFβRII and truncated CD19, which was included in receptor design for identification and selection (TCFβRII+CD19+: UT 1.92% ± 2.64% vs. RBDNR 43.9% ± 24.1% vs. NKA 43.2% ± 27.1% vs. NKCT 39.1% ± 26.3%, CD19+: UT 1.86% ± 3.57% vs. RBDNR 42.6% ± 27.6%, vs. NKA 43.9% ± 30.2% vs. NKCT 36.9% ± 29.4%, n > 30; FIG. 2B). Transduced NK cells could be enriched by performing immunomagnetic sorting with CD19 microbeads to achieve >90% enrichment (data not shown). Staining for natural cytotoxicity receptors NKp44 and NKp30 showed no significant difference in expression on transduced NK cells compared with their untransduced counterparts (NKp44: UT 27.40% ± 15.6% vs. RBDNR 25.1% ± 18.0% vs. NKA 31.9% ± 14.9% vs. NKCT 26.4% ± 18.2% P> 0.05, NKp30: UT 41.1% ± 27.7% vs. RBDNR 44.2% ± 28.9% vs. NKA 41.7% ± 26.5% vs. NKCT 41.9% ± 31.4% P > 0.05, n > 5, FIG. 14C). Similarly, no impairment in the expression of other NK-cell surface markers NKG2D, CD69, CD16, or PD1 was found (P > 0.05, n > 5; FIG. 14C). NK cells were labeled with CFSE and cocultured with unlabeled modified K562s. Flow cytometric analysis of CFSE dilution over three days demonstrated no changes in NK-cell proliferation after transduction with RBDNR, NKA, or NKCT receptors (fold change compared with unstimulated; UT 75.3-fold vs. RBDNR 88.5-fold vs. NKA 41.3-fold vs. NKCT 64.2-fold, P > 0.05, n > 5, FIG. 14D; Supplementary FIG. S4). ⁵¹Cr-based cytotoxicity assays with untransduced and transduced NK cells showed maintained cytolysis of K562 target cells in all conditions (UT vs. RBDNR vs. NKA vs. NKCT P > 0.05, n > 5; FIG. 14E). Additional cytotoxicity assays with untransduced and transduced cells showed maintained cytolysis of HTLA230 neuroblastoma target cells in all conditions (UT vs. RBDNR vs. NKA vs. NKCT P > 0.05, n > 5; Supplementary FIG. S2). These results showed that introducing an engineered TGFβ receptor for any of the RBDNR, NKA, or NKCT constructs did not affect NK-cell phenotype and function.

### TGFβ Receptor Modification Protects NK Cells From Downstream Molecular Effects Of Exogenous TGFβ

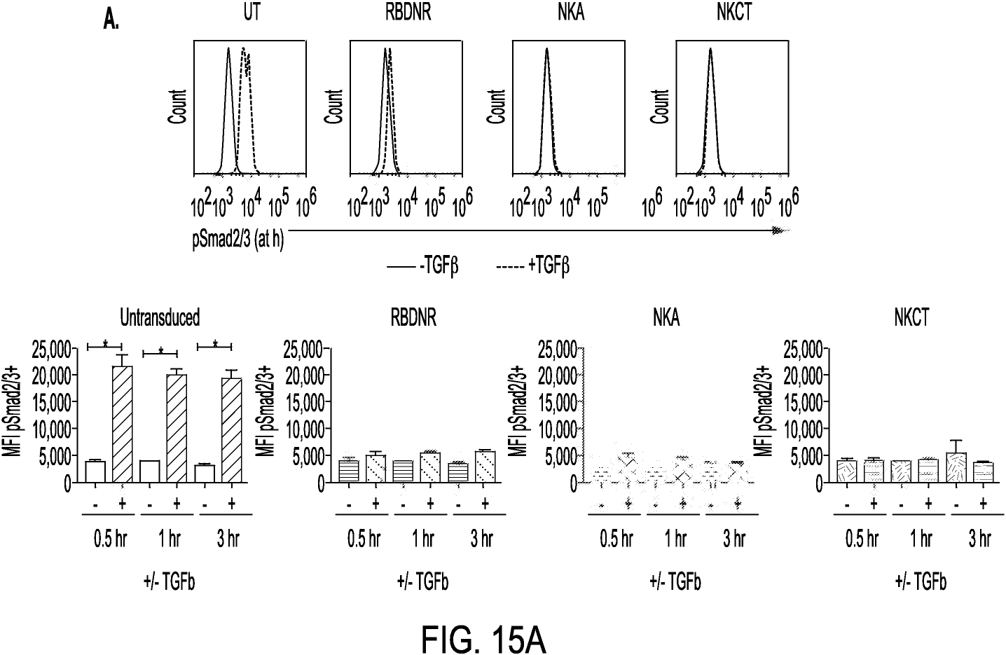

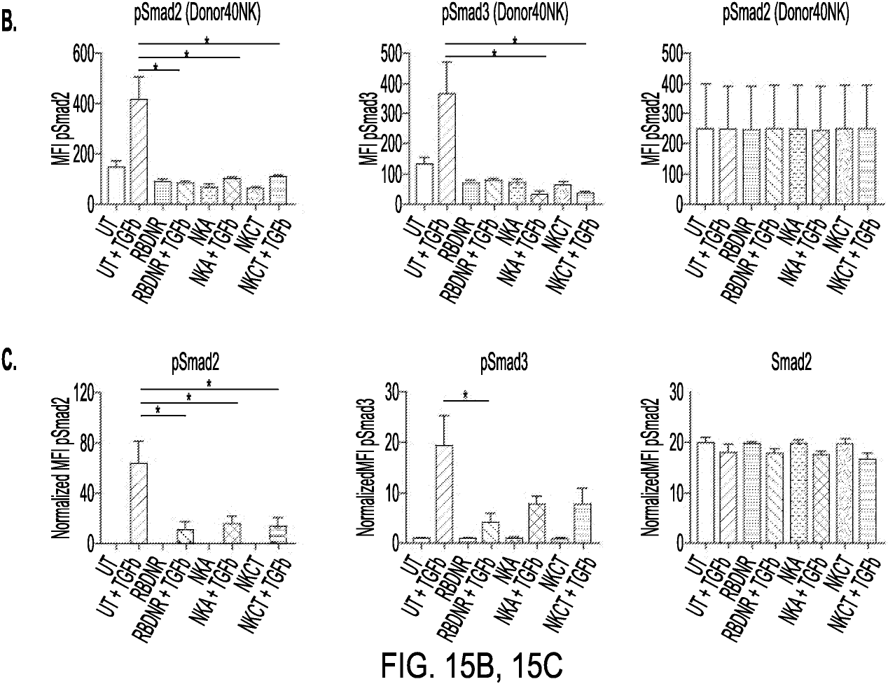

TGFβ binding initiates the phosphorylation of intracellular Smad2 and Smad3 proteins (15). To investigate the ability of RBDNR, NKA, and NKCT constructs to prevent TGFβ-mediated signaling, we cocultured untransduced, RBDNR, NKA, and NKCT-transduced NK cells with TGFβ. Cells were harvested 0.5, 1, or 3 hours after TGFβ exposure, and either assayed by flow cytometry or lysed to isolate and characterize intracellular proteins . Flow cytometry demonstrated rapid phosphorylation (Ser465/467) of Smad2/3 when untransduced NK cells were exposed to TGFfβ (pSmad2/3: UT+1.36 ± 0.95% vs. UT+TGFβ UT 73.9 ± 20.5%, P = 0.04 at 1 hour, n > 3; FIG. 3A), but not in NK cells transduced with either RBDNR, NKA, or NKCT receptors following TGFβ exposure (P > 0.05 at 1 hour, P>0.4 at 3 hours, n > 3; FIG. 3A). Similarly, evaluation of Smad2 (Ser465/467) and Smad3 (Ser423/425) phosphorylation from protein lysate isolated from untransduced and transduced cells after 1 hour of TGFβ exposure further demonstrated the protective effect of receptor modifications conferred to NK cells. Protein lysate results are shown from one representative NK line (FIG. 15B) as well as from pooled NK donor lines (pSmad2 UT+TGFβ vs. REDNR+TGFβ P= 0.025, UT+TGFβ vs. NKCT+TGFβ P = 0.031; pSmad3 UT+TGFβ vs. RBDNR+TGFβ P = 0.037, n > 5; FIG. 15C). These results demonstrated that Smad2 was only phosphorylated in UT NK cells exposed to TGFβ, while expression of the RBDNR, NKA, or NKCT receptors protected from Smad2 phosphorylation.

### TGFβ Receptor-Modified NK Cells Have Increased Expression of Activation Markers And Maintain Function in the Presence of TGFβ

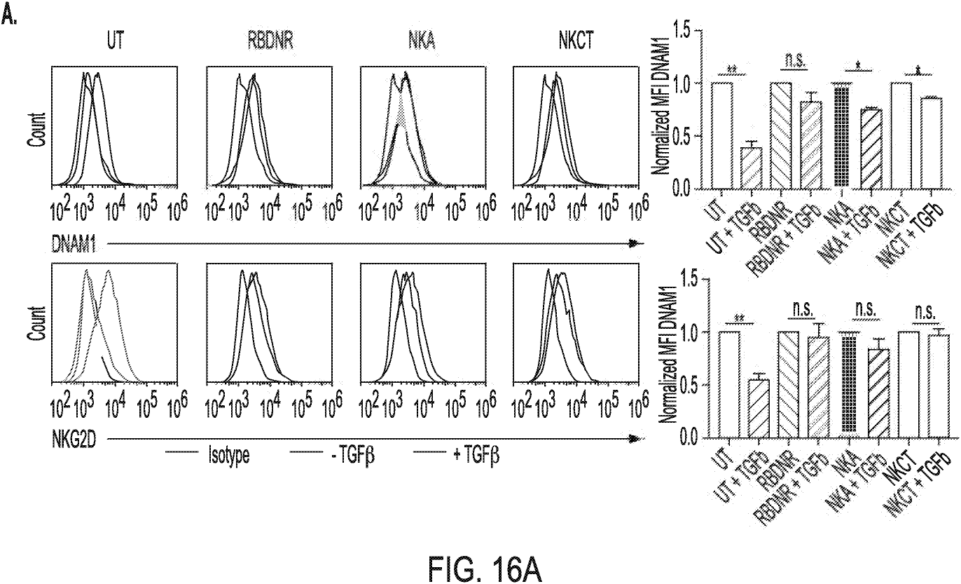

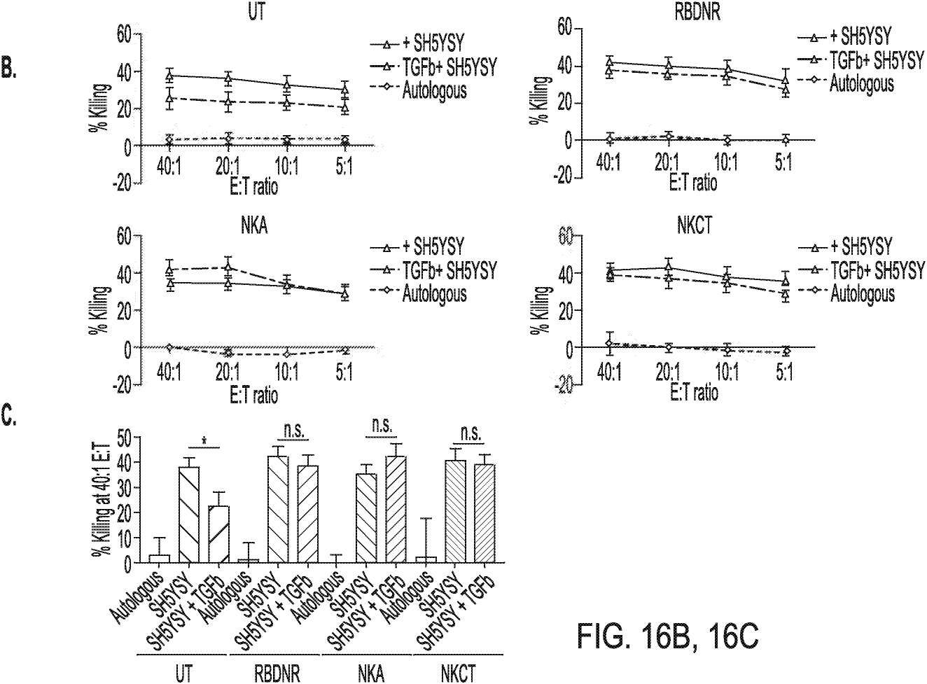

To assess whether the protection from the molecular changes occurring after TGFβ exposure translated to a phenotypic or functional advantage, untransduced, and RBDNR, NKA, and NKCT-transduced NK cells were examined after 5 days in culture with TGFβ. Flow cytometry showed decreased expression of DNAX Accessory Molecule-1 (DNAM1 fold change from non-TGFβ exposed: UT 0.39-fold, P ::: 0.0163, n > 5; FIG. 16A) and in NKG2D (fold-change from non- TGFβ exposed: UT 0.58-fold, P = 0.04, n > 5; FIG. 16A) in untransduced NK cells following exposure to TGFβ. Surface marker downregulation was not observed in RBDNR, NKA, or NKCT-transduced NK cells, which all exhibited protection from these TGFβ-mediated phenotype impairments (P > 0.05, n > 5; FIG. 4A). In addition, expression of CD16 was not impaired in transduced cells following TGFβ exposure, alluding to their potential to successfully mediate an antitumor effect via ADCC as well as cytolysis (Supplementary FIG. S5). Indeed, whereas untransduced NK cells showed dose-dependent cytotoxicity against SHSY5Y neuroblastoma cells (38.2% ± 4.69% killing at E:T ratio 40:1), they exhibited impaired cytolytic activity (24.6% ± 4.58% killing at E:T ratio 40: 1) following preculture with TGFβ (FIGS. 16B and C). Impaired cytolytic activity was not demonstrated when NK cells transduced to express the variant TGFβ receptors (RBDNR, NKA, or NKCT) were evaluated following pretreatment with TGFβ (FIGS. 16B and C), suggesting their functional superiority at killing target cells in a TGFβ-rich environment. As such, we found that not only did expression of the modified TGFβ receptors protect from the molecular signaling occurring in endogenous NK cells following TGFβ exposure, but this protection translated to a protection from altered phenotype and decreased antitumor activity occurring in untransduced cells exposed to TGFβ.

### DAP12 and RELA-Containing TGFβ Receptor-Variant NK Cells Demonstrated Increased Expression of Molecular Activation Markers Following Exposure to TGFβ

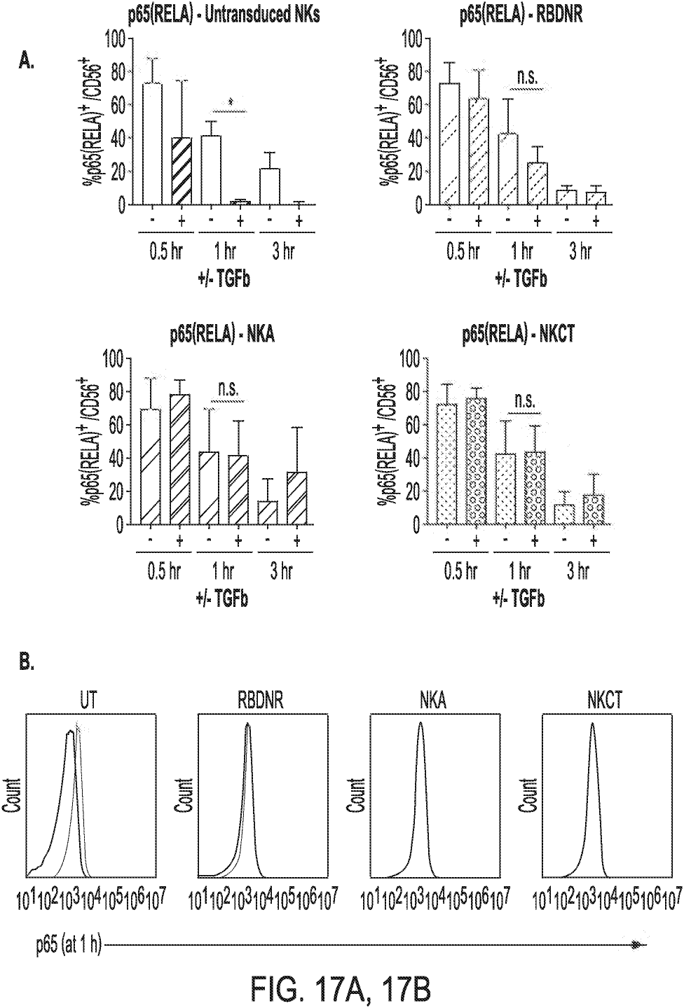

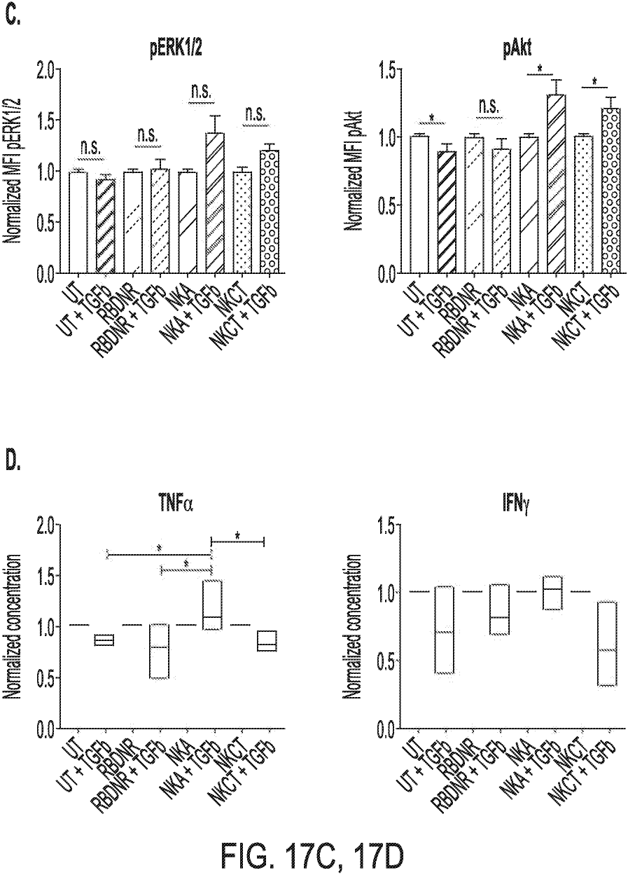

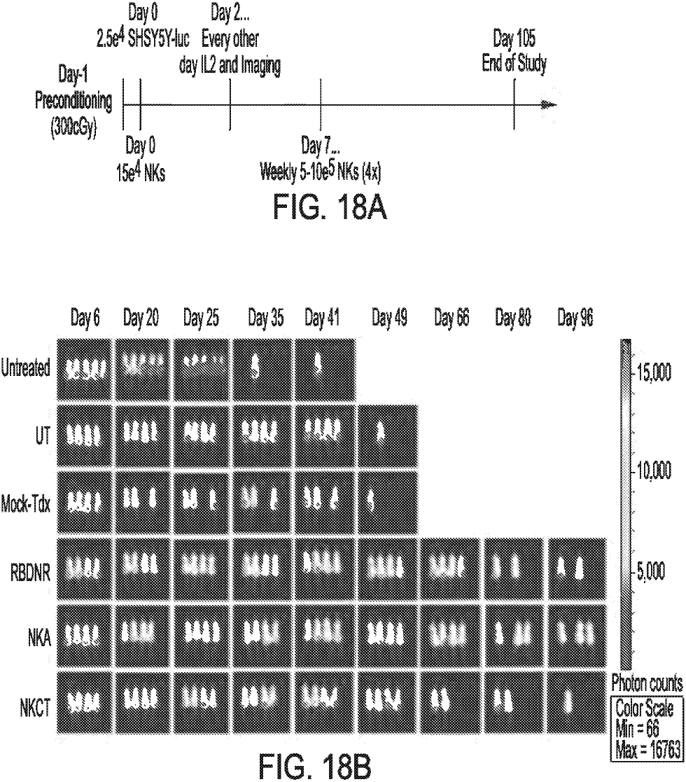

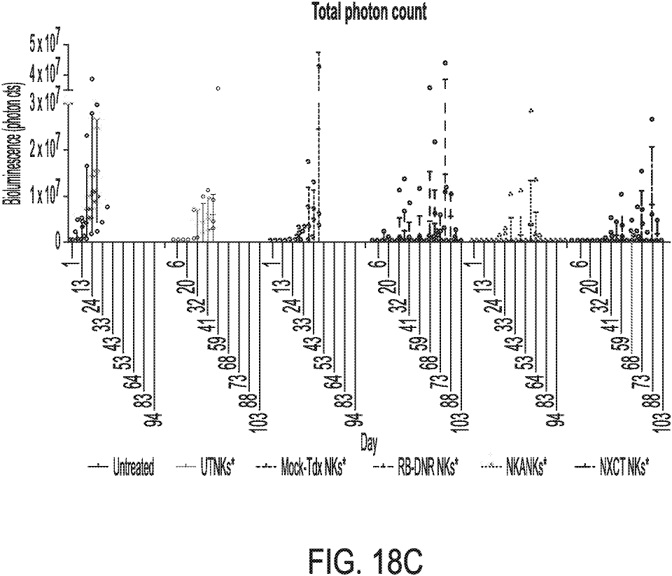

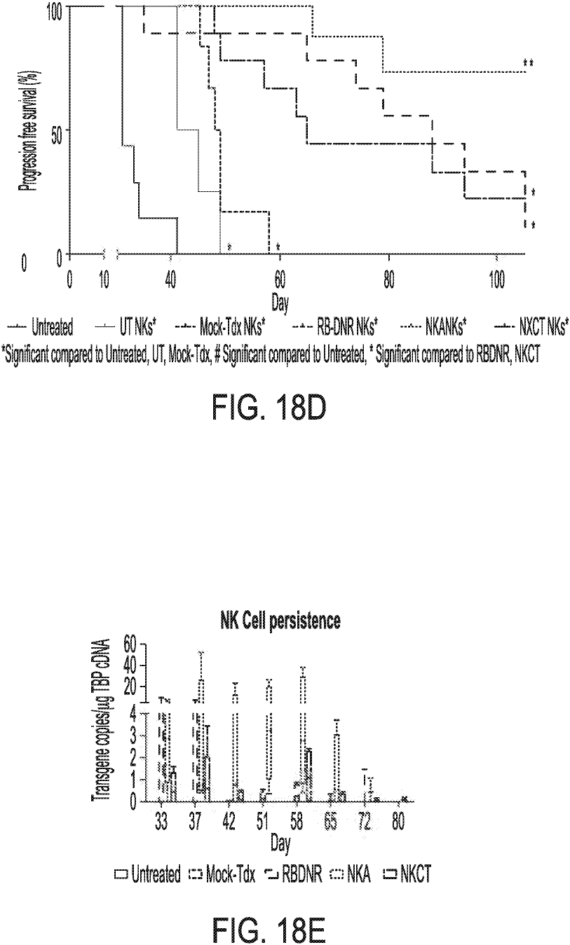

To examine the induction of NK-cell activation, we cocultured untransduced, RBDNR, NKA, and NKCT-transduced NK cells with TGFβ. Cells were harvested 0.5, 1, or 3 hours after TGFβ exposure and either lysed to isolate protein or assayed by flow cytometry. Using flow cytometry, we demonstrated decreasing levels of RELA (p65) in untransduced NK cells at 1 and 3 hours post TGFβ-exposure (UT 42.3% ± 13.7% vs UT+TGFβ UT 2.02% ± 1.08%, P = 0.02 at 1 hour UT 21.5% ± 11.5% vs. UT+TGFβ UT 0.47% ± 0.46%, P = 0.18 at 3 hours, n > 3; FIGS. 17A and B). Similar trends in RELA were seen in RBDNR-transduced NK cells at 1-hour post-TGFβ exposure (P = 0.31 at 1 hour, P = 0.18 at 3 hours, n > 3; FIGS. 17A and B) NK cells transduced with either NKA or NKCT-variant TGFβ receptors demonstrated unaltered p65 expression following exposure to TGFβ (NKA P = 0.92 at 1 hour. P = 0.61 and 3 hours, n > 3; NKCT P = 0.96 at 1 hour, P = 0.75 at 3 hours, n > 3), suggesting that NFKB-mediated signaling persisted in these cells. Evaluation of ERK1/2 (ThrI85/Tyr187) and Akt (Ser473) phosphorylation occurring in protein lysate isolated from untransduced and transduced cells after 1 hour of TGFβ exposure further showed activation in NKA and NKCT-transduced NK cells. While untransduced and RBDNR-transduced NK cells exhibited decreased or unchanged levels of Akt phosphorylation (UT vs. UT+TGFβ P = 0.0075, RBDNR vs. RBDNR+TGFβ P = 0.282, n > 5; FIG. 5C), NK cells equipped with the activation-inducing TGFβ variants had increased Akt phosphorylation (NKA vs. NKA > TGFβ P = 0.0013, NKCT vs. NKCT+TGFβ P = 0.0037, n > 5; FIG. 17C). In an examination of supernatant isolated from cell cultures after 12 hours of exposure to TGFβ, we found significantly increased TNFα production in NKA-transduced NK cells after cytokine exposure, as compared with either untransduced or other variant transduced NK-cell groups (NKA+TGFβ vs. UT+TGFβ P = 0.039, NKA+TGFβ vs. RBDNR+TGFβ P = 0.006, NKA+TGFβ vs. NKCT+TGFβ P = 0.041; FIG. 17D). Taken together, these results suggest that NK cells transduced to express the TGFβ receptor variants, in particular the NKA-modified receptor, demonstrated heightened NK activation, consistent with our observed molecular changes occurring along the NFKB and PI3K signaling pathways.

### Repeat Dosing With TGFβ Receptor-Modified NK Cells Enhances Survival and Tumor Eradication in a Xenograft Model of TGFβ Secreting Neuroblastoma

We established a xenograft model of human neuroblastoma using SHSY5Y human neuroblastoma cells (51), inoculated subcutaneously in preconditioned immunodeficient animals. Animals were randomly assigned to six treatment groups: untreated, untransduced NK cells (UT), mock GFP-transduced NK cells (Mock-Tdx), RBDNR-transduced NK cells (RBDNR), NKA-transduced NK cells (NKA), and NKCT-transduced NK cells (NKCT). After inoculation, animals were treated systemically (43) with 1.5 × 10⁷ NK cells, and monitored during alternate day intraperitoneal IL2 administration for the duration of the study. Repeated doses of untransduced or transduced NK cells were subsequently given on days 0, 7, 14, 21, and 28 following tumor inoculation (FIG. 6A), which mirrors desired clinical dosing regimens Tumor growth was monitored every other day by quantifying bioluminescence (total photon counts) of animals imaged with the IVIS system, using a normalized photon scale (52. 53). Rapid tumor progression was seen in untreated animals, who had a median survival of 31 days (FIGS. 18B and C). Animals infused with untransduced or mock-transduced NK cells showed delayed tumor progression compared with untreated animals; however, these animals eventually succumbed to tumor progression (UT median survival = 43 days, Mock-tdx median survival = 48.5 days; FIGS. 18B and C). In contrast, infusion of RBDNR or NKCT-transduced NK cells led to improved tumor control and prolonged survival (RBDNR median survival = 88 days, NKCT median survival = 65 days; survival untreated vs. RBDNR P < 0.0001, untreated vs. NKCT P < 0.0001; FIG. 6D). Animals treated with NKA-transduced NK cells exhibited superior protection from tumor progression (FIGS. 6B and C; Supplementary FIG. S6) and significantly enhanced survival (progression-free survival = 72.9%, survival untreated vs. NKA P < 0.0001, UT vs. NKA P = 0.0001, RBDNR vs. NKA P = 0.0333, NKCT vs. NKA P = 0.0313; FIG. 6D). In an assessment to determine the immune populations in the peripheral blood of mice using flow cytometry, we showed that NK cells represented a very minor (<1%) population of the total lymphoid compartment (Supplementary FIG. S7), and as such, the more sensitive ddPCR assay was used to identify the presence of unmodified or modified NK cells peripherally. Therefore, peripheral blood was isolated weekly following the final therapeutic dose of NK cells on day 28, and RNA was extracted from the blood to evaluate the presence of the NK-cell transgene (GFP or TGFβ variant receptor) by quantitative ddPCR assay. At 5 and 9 days after the final infusion, modified NK cells were identified in circulation. Over the next 6 weeks, there was some evidence of RBDNR and NKCT-transduced NK cells persisting, although in progressively dwindling numbers as time continued and tumors progressed (FIG. 6E; Supplementary Table S1). NKA-transduced NK cells, however, persisted in higher frequencies than either RBDNR or NKCT-transduced NK. cells (FIG. 17E). Analysis of the TBP transgene in all samples ensured a sufficient quantity and quality of DNA, and was used to normalize all results.

Taken together, these data indicate that NK cells modified to express novel variants of a TGFβ receptor protect cells from the inhibitory effects of neuroblastoma-associated TGFβ and demonstrate superior antitumor efficacy in vivo. Furthermore, the enhanced persistence of NKA-transduced NK cells and the significant improvement in progression-free survival in mice administered NKA-transduced NK cells over the RBDNR- and NKCT-transduced NK-cell products suggest that coupling the TGFβ receptor modification to the NK-specific signaling motif DAP12 confers additional therapeutic advantages and prolonged NK-cell persistence in vivo.

### Discussion

In this study, we genetically engineered NK cells with novel TGFβ receptors to counter any suppressive TGFβ-mediated signaling and investigated whether we could switch the negative TGFβ signal into an activating signal. We demonstrated that phosphorylation of Smad2 and Smad3 occurred as early as 30 minutes after TGFβ exposure in unmodified NK cells, but was blocked in RBDNR-, NKA-, and NKCT-transduced NK cells. The signaling cascade initiated by the phosphorylation of Smad2/3 led to impaired expression of surface receptors (54) and consequent impairment of antitumor cytolytic function. We found that not only were cord blood-modified NK cells resistant to the inhibitory effects of tumor-associated TGFβ-they also showed superior antitumor efficacy in a TGFβ-rich tumor setting, specifically when transduced with the NKA receptor. The strategy of rendering cell therapy products resistant to inhibitory TGFβ has been explored in a number of malignancies (19, 23). However, by fully inactivating the negative TGFβ pathway and converting the inhibitory signal to an ancillary signal, we created a novel and potent NK-cell-specific therapeutic which could be used as an allogeneic “off the shelf” cellular therapy for the treatment of patients with neuroblastoma.

Use of the synthetic Notch receptor into the NKCT receptor is a strategy conceptualized and first applied in the setting of chimeric antigen receptor generation for T cells (26, 27). This strategy employs logic gating, requiring the cell to receive a primary signal to trigger a secondary signal through a “SynNotch” receptor. The “SynNotch” receptor contains a core regulatory Notch domain, coupled to an intracellular transcriptional domain that cleaves and engages with nuclear promoters to initiate a given transcriptional change. The NKCT receptor used here contains the extracellular TGFβ dominant-negative receptor coupled to a Notch and RELA-linked domain; engagement of TGFβ with this receptor would trigger cleavage of the “SynNotch” motif leading to increased transcription of RELA (p65) and consequent increase in NK-cell activation. Our in vitro experiments with the NKCT construct validated this strategy for activating NK cells. However, the potential advantage of this construct was not borne out in vivo, as systemic treatment with NKCT-modified NK cells achieved antitumor efficacy and progression-free survival no better than achieved by RBDNR-modified NK cells that only block TGFβ-mediated signaling. The size of the construct might have been a limiting factor, impairing cleavage and translocation of the large intracellular signaling portion of this receptor. In addition, because the construct bypassed a natural signaling cascade instead of leading directly to transcriptional activation, it is possible that in the TGFβ-rich environment NKCT-transduced NK cells could be chronically activated causing NK-cell dysfunction and apoptosis. Alternatively, chronic activation could have generated a negative feedback loop from inhibitory cytokines (55).

In contrast, the NKA receptor (containing DAP12 fused to the dominant-negative receptor facilitating NK-specific intracellular signaling) led to improved activity in vivo. In unmodified NK cells, DAP12 associates with natural activating and cytotoxicity receptors, such as NKG2C and NKp44. Once dimerized, the ITAM-containing cytoplasmic domain can readily dock with Zap70 and Syk proteins. Global cell activation is the resultant effect of DAP12 activation, which signals through the P13K/ERK and Akt pathways (25, 56-58) By incorporating the transmembrane and ITAM-containing domains of DAP12 in the NKA construct, TGFβ binding with the engineered receptor triggered activation of DAP12 signaling and enhanced the NK-cell activity. The antitumor efficacy of the NKA construct was superior to that obtained with NK cells engineered only to block TGFβ signaling, as in the RBDNR-engineered cells. Furthermore, this additional modification conferred a distinct survival advantage, with NKA-transduced cells persisting up to 7 weeks following their final infusion in treated animals. This in turn led to a superior antitumor effect and a survival advantage in these mice. Further assessment of activation markers expressed by NK cells isolated ex vivo from treated animals would allow a greater depth of understanding into the in vivo mechanism, and will be an important component of larger scale efforts as this approach is translated to the clinic. Although the enhanced PBK/Akt signaling found in vitro indicates successful propagation of DAP12-mediated activation, it does not specifically address the mechanism through which the TGFβRII-DAP12-linked receptor is forming a dimer or tetramer, and the resultant signaling cascade As such, it would be essential for future studies to further elucidate this signaling mechanism as well as examine other downstream molecular targets to ensure that enhanced Akt activity would not lead to artificially enhanced NK-cell exhaustion.

Topfer and colleagues have also incorporated DAP12 signaling into a prostate stem cell antigen (PSCA)-specific CAR construct. Preliminary results confirmed the benefit of the DAP12 construct over non-DAPI2-containing CAR cells (39); however, this effort was conducted with the NK-cell line YTS, which, although similar to endogenous NK cells in phenotype, lacks the KIR expression resident to primary NK cells. Our efforts genetically modifying primary NK cells derived from cord blood sources represents a clinically relevant application, where interaction between inhibitory KIRs on NK cells with MHC 1 variants can have a large influence on the resultant activity (cytotoxicity or suppression) of NK cells used for cell therapy. Our modification of NK cells with a combination of enhanced cell activity through DAP12 and ameliorated TCFβ blockade represents a novel and promising cell therapy approach for neuroblastoma and other malignancies.

One drawback of using CD19 expression to identify transduced cells is that selective downregulation of either the TGFβ-modified receptor or the CD19 tag could occur. By using immunomagnetic beads to selectively enrich our cell populations, we minimized the likelihood of this happening (Supplementary FIG. S3). While engineered NK cells might downregulate the modified TGFβ receptor over time, our in vivo studies identified gene-modified NK cells with biological activity beyond four weeks suggesting that the cell constructs were stable and could exert long-term antitumor effects.

This report demonstrates preclinical efficacy of a novel mechanism to convert a customarily inhibitory signal, TGFβ, into an activating pathway for NK cells—by doing so, the TGFβ-rich tumor microenvironment is transformed to enhance NK-cell....mediated cytotoxicity of tumors. By generating NK-cell products from over 30 umbilical cord blood units, and through in vitro and in vivo testing in a human xenograft model of neuroblastoma, this report supports translation to clinical applications. Further preclinical work is being pursued to identify the potential mechanisms of escape that could be faced clinically. For example, examining the function of these variant TGFβ receptors in a humanized model would be of considerable future interest because humanized neuroblastoma models would provide the opportunity to examine interactions with other immune components (e.g., myeloid-derived suppressor cells) that may also play a role in promoting NK-cell dysfunction in the neuroblastoma setting . Furthermore, ex vivo profiling of immune subsets over time would allow for further in depth analysis of the interactions between NK cells and other immune effectors, and could help determine whether the gene engineered NK cells are capable of eliciting enhanced cytotoxicity through supporting ADCC in addition to tumor-targeted cytotoxicity. Although many neuroblastomas have decreased or absent levels of MHC I, rendering them attractive targets for NK-mediated cytolysis, it would also be of considerable interest in further studies to examine the efficacy of this NK-based immunotherapy in a tumor that has upregulated MHC I expression as a method of tumor escape. In such a setting, however, combining NK-cell therapy with other immunomodulatory agents (small molecule or epigenetic) may represent an attractive therapeutic avenue. Finally, another priority in further preclinical testing and in initial clinical readouts would be to determine the extent of NK-cell migration to tumor-draining lymph nodes and other biological niches following repeat NK-cell dosing. While preliminary efforts revealed that CCR2 expression is impaired in NK cells following exposure to TGFβ, and modification with the dominant negative receptor (and variants) may protect from this decline, further probing of the complete effect on NK cells migration is the subject of future study.

In summary, cord blood-derived NK cells modified to avoid the inhibitory effects of TGFβ represent an efficient way to harness fast-acting innate immune cells for therapy. Furthermore, our development of novel variant TGFβ receptors, composed of the dominant-negative receptor coupled to intracellular signaling domains initiating NK-cell activation, represents a unique approach to transform a classical tumor-inhibitory mechanism into a therapeutic weapon. Our preclinical results support translational research to establish allogeneic, cord blood-derived, gene-modified NK cells to treat patients with neuroblastoma and other malignancies that use TGFβ secretion as a potent immune evasion mechanism.

1. A human cell comprising:
(a) a first exogenous nucleic acid sequence comprising a sequence encoding a fusion protein comprising a first and a second domain, wherein the first domain comprises an extracellular TGF-β receptor sequence capable of binding TGF-β and the second domain comprises an intracellular signaling sequence that is free of a biologically active TGF-β receptor I (TGF-βRI) or a modified TGF-β receptor II (TGF-PRII) intracellular domain, wherein the intracellular signaling sequence comprises an NK cell activation domain or sequence;
(b) a second exogenous nucleic acid sequence comprising a sequence encoding one or more cytokines; and
(c) a third exogenous nucleic acid sequence comprising a sequence encoding a chimeric antigen receptor (CAR).

2. The human cell of claim 1, wherein the human cell is a primary antigenic presenting cell. T-cell, or NK cell.

3. The human cell of claim 1 , wherein the human cell is a primary NK cell harvested from a subject or a human cell derived from an umbilical cord blood of a subject.

4. (canceled)

5. (canceled)

6. (canceled)

7. (canceled)

8. (canceled)

9. (canceled)

10. (canceled)

11. (canceled)

12. The human cell of claim 1, wherein the human cell comprises a viral vector that comprises one or more of the first exogenous nucleic acid sequence, the second exogenous nucleic acid sequence, and the third exogenous nucleic acid sequence.

13. (canceled)

14. (canceled)

15. The human cell of claim 1, wherein the chimeric antigen receptor comprises an amino acid sequence that binds to a cancer cell.

16. (canceled)

17. The human cell of claim 1, wherein the one or more cytokines are selected from the group consisting of: IL-2, IL-12, IL-15, IL-18, IL-21, and IL-27.

18. (canceled)

19. The human cell of claim 1, wherein the extracellular TGF-β receptor sequence comprises an extracellular portion of human TGFβ-RI or an extracellular portion of human TGFβ-RII.

20. The human cell of claim 1, wherein the second amino acid domain comprises a functional fragment of one or more polypeptides selected from the group consisting of: DAP-12, KIR2DS1, KIR2DS2, KIR2DS3, KIR2DS4, KIR2DS5, KIR3DS1, NKp44, NKG2C, NKG2E, NOTCH1, NOTCH2, NOTCH3, and NOTCH4.

21. (canceled)

22. A pharmaceutical composition comprising: (i) a therapeutically effective amount of the human cells of claim 1; and (ii) a pharmaceutically acceptable carrier.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A method for inducing cell death of a target cell, the method comprising:
contacting the human cell of claim 1 with a target cell.

35. (canceled)

36. (canceled)

37. (canceled)

38. (canceled)

39. (canceled)

40. (canceled)

41. A method of treating cancer in a subject in need thereof, the method comprising administering to the subject a therapeutically effective amount of the human cells of claim 1.

42. A method of treating a hyperproliferative disorder characterized by dysfunctional expression of TGFβ in a subject in need thereof, the method comprising administering to the subject a therapeutically effective amount of the human cells of claim 1.

43. A method of preventing progression of cancer in a subject in need thereof, the method comprising administering to the subject a therapeutically effective amount of the human cells of claim 1.

44. A method of targeting and/or killing a hyperproliferative cell in a subject, the method comprising administering to the subject a therapeutically effective amount of the human cells of claim 1.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. The human cell of claim 1, wherein the fusion protein comprises a transmembrane domain of DAP-12, KIR2DS1, KIR2DS2, KIR2DS3, KIR2DS4, KIR2DS5, KIR3DS1, NKp44, NKG2C, or NKG2E.

59. A human cell comprising an exogenous nucleic acid sequence comprising a sequence encoding a fusion protein comprising a first and a second domain, wherein the first domain comprises an extracellular TGF-β receptor sequence capable of binding TGF-β and the second domain comprises an intracellular signaling sequence that is free of a biologically active TGF-P receptor I (TGF-PRI) or TGF-β receptor II (TGF-PRII) intracellular domain, and the second domain comprises a functional fragment of one or more polypeptides selected from the group consisting of: KIR2DS1, KIR2DS2, KIR2DS3, KIR2DS4, KIR2DS5, KIR3DS1, NKp44, NKG2C, NKG2E, NOTCH1, NOTCH2, NOTCH3, and NOTCH4.

60. The human cell of claim 59, wherein the human cell is a primary antigenic presenting cell, T-cell, or NK cell.

61. A human cell comprising an exogenous nucleic acid sequence comprising a coding sequence, wherein the coding sequence comprises a nucleic acid sequence that is at least 90% identical to the nucleic acid sequence of SEQ ID NO: 4 and a nucleic acid sequence that is at least 90% identical to the nucleic acid sequence of SEQ ID NO: 9.


SEQUENCE LISTING (39 sequences)

SEQ ID NO: 1 - DNA, 54 bp, Homo sapiens
gagggcagaggctccctgctgacctgcggcgatgtggaggagaatccaggacct

SEQ ID NO: 2 - DNA, 999 bp, Homo sapiens
atgcctccaccaaggctgctgttctttctgctgttcctgacaccaatggaggtgcggcccgaggagcctctggtggtgaaggtggaggagggcgacaacgccgtgctgcagtgtctgaagggcacctctgatggccccacccagcagctgacatggtctagggagagcccactgaagccctttctgaagctgagcctgggcctgccaggcctgggtatccacatgcgccctctggccatctggctgttcatcttcaacgtgagccagcagatgggaggcttctacctgtgccagccaggacctccatctgagaaggcctggcagcctggatggaccgtgaacgtggagggaagcggagagctgtttcggtggaacgtgagcgacctgggaggcctgggatgtggcctgaagaacagatcctctgagggccctagctccccatctggcaagctgatgagcccaaagctgtacgtgtgggccaaggataggccagagatctgggagggagagccaccttgcctgccaccccgcgactccctgaatcagtccctgtctcaggatctgacaatggcccctggctccaccctgtggctgtcttgtggcgtgcctccagacagcgtgtccagaggcccactgtcttggacccacgtgcaccccaagggccctaagtccctgctgtctctggagctgaaggacgatcggcctgccagagacatgtgggtcatggagacaggcctgctgctgccacgggccaccgcacaggatgccggcaagtactattgccacagaggcaacctgacaatgagcttccacctggagatcaccgcccggcccgtgctgtggcactggctgctgagaacaggcggctggaaggtgtctgccgtgaccctggcctacctgatcttctgcctgtgcagcctggtgggcatcctgcacctgcagagggccctggtgctgaggagaaagaggaagcgcatgaccgaccctacaaggcgcttt

SEQ ID NO: 3 - DNA, 66 bp, Homo sapiens
atgggaaggggcctgctgagaggcctgtggcccctgcacatcgtgctgtggaccaggatcgcctcc

SEQ ID NO: 4 - DNA, 432 bp, Homo sapiens
acaatcccccctcatgtgcagaagtctgtgaacaatgacatgatcgtgacagataacaatggcgccgtgaagttccctcagctgtgcaagttctgtgacgtgcggtttagcacatgcgataaccagaagtcctgcatgtctaattgtagcatcacctccatctgcgagaagccacaggaggtgtgcgtggccgtgtggagaaagaacgacgagaatatcaccctggagacagtgtgccacgatcctaagctgccataccacgactttatcctggaggatgccgccagccctaagtgtatcatgaaggagaagaagaagccaggcgagacattcttcatgtgctcctgtagctccgacgagtgtaacgataatatcatcttcagcgaggagtataacacatccaatccagacctgctgctggtcatctttcag

SEQ ID NO: 5 - DNA, 63 bp, Homo sapiens
gtgacaggcatcagcctcctgccaccactgggagttgccatatctgtcatcatcatcttctac

SEQ ID NO: 6 - DNA, 36 bp, Homo sapiens
tgctaccgcgttaaccggcagcagaagctgagttca

SEQ ID NO: 7 - DNA, 57 bp, Homo sapiens
ctgcgcccagtgcaggcacaggcacagtctgactgctcttgtagcacagtgagccca

SEQ ID NO: 8 - DNA, 63 bp, Homo sapiens
ggcgtgctggcaggaatcgtgatgggcgatctggtgctgaccgtgctgatcgccctggccgtg

SEQ ID NO: 9 - DNA, 156 bp, Homo sapiens
tactttctgggccggctggtgcctcggggcagaggagcagcagaggcagccaccaggaagcagcgcatcaccgagacagagagcccctaccaggagctgcagggccagaggagcgacgtgtattccgatctgaacacacagcgcccttactataag

SEQ ID NO: 10 - DNA, 48 bp, Homo sapiens
tcacccactgaaccaagctccaaaaccggtaaccccagacacctgcat

SEQ ID NO: 11 - DNA, 63 bp, Homo sapiens; identical to SEQ ID NO: 8

SEQ ID NO: 12 - DNA, 156 bp, Homo sapiens; identical to SEQ ID NO: 9

SEQ ID NO: 13 - DNA, 2016 bp, Homo sapiens
atgggaaggggcctgctgagaggcctgtggcccctgcacatcgtgctgtggaccaggatcgcctccacaatcccccctcatgtgcagaagtctgtgaacaatgacatgatcgtgacagataacaatggcgccgtgaagttccctcagctgtgcaagttctgtgacgtgcggtttagcacatgcgataaccagaagtcctgcatgtctaattgtagcatcacctccatctgcgagaagccacaggaggtgtgcgtggccgtgtggagaaagaacgacgagaatatcaccctggagacagtgtgccacgatcctaagctgccataccacgactttatcctggaggatgccgccagccctaagtgtatcatgaaggagaagaagaagccaggcgagacattcttcatgtgctcctgtagctccgacgagtgtaacgataatatcatcttcagcgaggagtataacacatccaatccagacctgctgctggtcatctttcaggtgaccggaatctctctgctgccaccactgggagtggcaatcagcgtgatcatcatcttctactgctatcgggtgaacagacagcagaagctgtctagcatgggcggcctggagccttgtagcaggctgctgctgctgccactgctgctggccgtgtccggcctgcgcccagtgcaggcacaggcacagtctgactgctcttgtagcacagtgagcccaggcgtgctggcaggaatcgtgatgggcgatctggtgctgaccgtgctgatcgccctggccgtgtactttctgggccggctggtgcctcggggcagaggagcagcagaggcagccaccaggaagcagcgcatcaccgagacagagagcccctaccaggagctgcagggccagaggagcgacgtgtattccgatctgaacacacagcgcccttactataagggatctggaggaagcggaggatccggagagggcagaggctccctgctgacctgcggcgatgtggaggagaatccaggacctatgcctccaccaaggctgctgttctttctgctgttcctgacaccaatggaggtgcggcccgaggagcctctggtggtgaaggtggaggagggcgacaacgccgtgctgcagtgtctgaagggcacctctgatggccccacccagcagctgacatggtctagggagagcccactgaagccctttctgaagctgagcctgggcctgccaggcctgggcatccacatgcgccctctggccatctggctgttcatcttcaacgtgagccagcagatgggaggcttctacctgtgccagccaggacctccatctgagaaggcctggcagcctggatggaccgtgaacgtggagggaagcggagagctgtttcggtggaacgtgagcgacctgggaggcctgggatgtggcctgaagaacagatcctctgagggccctagctccccatctggcaagctgatgagcccaaagctgtacgtgtgggccaaggataggccagagatctgggagggagagccaccttgcctgccaccccgcgactccctgaatcagtccctgtctcaggatctgacaatggcccctggctccaccctgtggctgtcttgtggcgtgcctccagacagcgtgtccagaggcccactgtcttggacccatgtgcaccccaagggccctaagtccctgctgtctctggagctgaaggacgatcggcctgccagagacatgtgggtcatggagacaggcctgctgctgccacgggccaccgcacaggatgccggcaagtactattgccacagaggcaacctgacaatgagcttccacctggagatcaccgcccggcccgtgctgtggcactggctgctgagaacaggcggctggaaggtgtctgccgtgaccctggcctacctgatcttctgcctgtgcagcctggtgggcatcctgcacctgcagagggccctggtgctgaggagaaagaggaagcgcatgaccgaccctacaaggcgcttt

SEQ ID NO: 14 - DNA, 1767 bp, Homo sapiens
acaatcccccctcatgtgcagaagtctgtgaacaatgacatgatcgtgacagataacaatggcgccgtgaagttccctcagctgtgcaagttctgtgacgtgcggtttagcacatgcgataaccagaagtcctgcatgtctaattgtagcatcacctccatctgcgagaagccacaggaggtgtgcgtggccgtgtggagaaagaacgacgagaatatcaccctggagacagtgtgccacgatcctaagctgccataccacgactttatcctggaggatgccgccagccctaagtgtatcatgaaggagaagaagaagccaggcgagacattcttcatgtgctcctgtagctccgacgagtgtaacgataatatcatcttcagcgaggagtataacacatccaatccagacctgctgctggtcatctttcagctgcgcccagtgcaggcacaggcacagtctgactgctcttgtagcacagtgagcccaggcgtgctggcaggaatcgtgatgggcgatctggtgctgaccgtgctgatcgccctggccgtgtactttctgggccggctggtgcctcggggcagaggagcagcagaggcagccaccaggaagcagcgcatcaccgagacagagagcccctaccaggagctgcagggccagaggagcgacgtgtattccgatctgaacacacagcgcccttactataagggatctgagggcagaggctccctgctgacctgcggcgatgtggaggagaatccaggacctatgcctccaccaaggctgctgttctttctgctgttcctgacaccaatggaggtgcggcccgaggagcctctggtggtgaaggtggaggagggcgacaacgccgtgctgcagtgtctgaagggcacctctgatggccccacccagcagctgacatggtctagggagagcccactgaagccctttctgaagctgagcctgggcctgccaggcctgggtatccacatgcgccctctggccatctggctgttcatcttcaacgtgagccagcagatgggaggcttctacctgtgccagccaggacctccatctgagaaggcctggcagcctggatggaccgtgaacgtggagggaagcggagagctgtttcggtggaacgtgagcgacctgggaggcctgggatgtggcctgaagaacagatcctctgagggccctagctccccatctggcaagctgatgagcccaaagctgtacgtgtgggccaaggataggccagagatctgggagggagagccaccttgcctgccaccccgcgactccctgaatcagtccctgtctcaggatctgacaatggcccctggctccaccctgtggctgtcttgtggcgtgcctccagacagcgtgtccagaggcccactgtcttggacccacgtgcaccccaagggccctaagtccctgctgtctctggagctgaaggacgatcggcctgccagagacatgtgggtcatggagacaggcctgctgctgccacgggccaccgcacaggatgccggcaagtactattgccacagaggcaacctgacaatgagcttccacctggagatcaccgcccggcccgtgctgtggcactggctgctgagaacaggcggctggaaggtgtctgccgtgaccctggcctacctgatcttctgcctgtgcagcctggtgggcatcctgcacctgcagagggccctggtgctgaggagaaagaggaagcgcatgaccgaccctacaaggcgcttt

SEQ ID NO: 15 - DNA, 1716 bp, Homo sapiens
acaatcccccctcatgtgcagaagtctgtgaacaatgacatgatcgtgacagataacaatggcgccgtgaagttccctcagctgtgcaagttctgtgacgtgcggtttagcacatgcgataaccagaagtcctgcatgtctaattgtagcatcacctccatctgcgagaagccacaggaggtgtgcgtggccgtgtggagaaagaacgacgagaatatcaccctggagacagtgtgccacgatcctaagctgccataccacgactttatcctggaggatgccgccagccctaagtgtatcatgaaggagaagaagaagccaggcgagacattcttcatgtgctcctgtagctccgacgagtgtaacgataatatcatcttcagcgaggagtataacacatccaatccagacctgctgctggtcatctttcagtcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcaggatctgagggcagaggctccctgctgacctgcggcgatgtggaggagaatccaggacctatgcctccaccaaggctgctgttctttctgctgttcctgacaccaatggaggtgcggcccgaggagcctctggtggtgaaggtggaggagggcgacaacgccgtgctgcagtgtctgaagggcacctctgatggccccacccagcagctgacatggtctagggagagcccactgaagccctttctgaagctgagcctgggcctgccaggcctgggtatccatatgcgccctctggcgatctggctgtttatcttcaacgtgagccagcagatgggaggcttctacctgtgccagccaggacctccatctgagaaggcctggcagcctggatggaccgtgaacgtggagggaagcggagagctgtttcggtggaacgtgagcgacctgggaggcctgggatgtggcctgaagaacagatcctctgagggccctagctccccatctggcaagctgatgagcccaaagctgtacgtgtgggccaaggataggccagagatctgggagggagagccaccttgcctgccaccccgcgactccctgaatcagtccctgtctcaggatctgacaatggcccctggctccaccctgtggctgtcttgtggcgtgcctccagacagcgtgtccagaggcccactgtcttggacccacgtgcaccccaagggccctaagtccctgctgtctctggagctgaaggacgatcggcctgccagagacatgtgggtcatggagacaggcctgctgctgccacgggccaccgcacaggatgccggcaagtactattgccacagaggcaacctgacaatgagcttccacctggagatcaccgcccggcccgtgctgtggcactggctgctgagaacaggcggctggaaggtgtctgccgtgaccctggcctacctgatcttctgcctgtgcagcctggtgggcatcctgcacctgcagagggccctggtgctgaggagaaagaggaagcgcatgaccgaccctacaaggcgcttt

SEQ ID NO: 16 - DNA, 8179 bp, Homo sapiens
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgggaaggggcctgctgagaggcctgtggcccctgcacatcgtgctgtggaccaggatcgcctccacaatcccccctcatgtgcagaagtctgtgaacaatgacatgatcgtgacagataacaatggcgccgtgaagttccctcagctgtgcaagttctgtgacgtgcggtttagcacatgcgataaccagaagtcctgcatgtctaattgtagcatcacctccatctgcgagaagccacaggaggtgtgcgtggccgtgtggagaaagaacgacgagaatatcaccctggagacagtgtgccacgatcctaagctgccataccacgactttatcctggaggatgccgccagccctaagtgtatcatgaaggagaagaagaagccaggcgagacattcttcatgtgctcctgtagctccgacgagtgtaacgataatatcatcttcagcgaggagtataacacatccaatccagacctgctgctggtcatctttcagctgcgcccagtgcaggcacaggcacagtctgactgctcttgtagcacagtgagcccaggcgtgctggcaggaatcgtgatgggcgatctggtgctgaccgtgctgatcgccctggccgtgtactttctgggccggctggtgcctcggggcagaggagcagcagaggcagccaccaggaagcagcgcatcaccgagacagagagcccctaccaggagctgcagggccagaggagcgacgtgtattccgatctgaacacacagcgcccttactataagggatctgagggcagaggctccctgctgacctgcggcgatgtggaggagaatccaggacctatgcctccaccaaggctgctgttctttctgctgttcctgacaccaatggaggtgcggcccgaggagcctctggtggtgaaggtggaggagggcgacaacgccgtgctgcagtgtctgaagggcacctctgatggccccacccagcagctgacatggtctagggagagcccactgaagccctttctgaagctgagcctgggcctgccaggcctgggtatccacatgcgccctctggccatctggctgttcatcttcaacgtgagccagcagatgggaggcttctacctgtgccagccaggacctccatctgagaaggcctggcagcctggatggaccgtgaacgtggagggaagcggagagctgtttcggtggaacgtgagcgacctgggaggcctgggatgtggcctgaagaacagatcctctgagggccctagctccccatctggcaagctgatgagcccaaagctgtacgtgtgggccaaggataggccagagatctgggagggagagccaccttgcctgccaccccgcgactccctgaatcagtccctgtctcaggatctgacaatggcccctggctccaccctgtggctgtcttgtggcgtgcctccagacagcgtgtccagaggcccactgtcttggacccacgtgcaccccaagggccctaagtccctgctgtctctggagctgaaggacgatcggcctgccagagacatgtgggtcatggagacaggcctgctgctgccacgggccaccgcacaggatgccggcaagtactattgccacagaggcaacctgacaatgagcttccacctggagatcaccgcccggcccgtgctgtggcactggctgctgagaacaggcggctggaaggtgtctgccgtgaccctggcctacctgatcttctgcctgtgcagcctggtgggcatcctgcacctgcagagggccctggtgctgaggagaaagaggaagcgcatgaccgaccctacaaggcgcttttaaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 17 - protein, 19 aa, Artificial Sequence
  Transmembrane peptide 1
VLIGTSVVKIPFTILLFFL

SEQ ID NO: 18 - protein, 20 aa, Artificial Sequence
  TRANSMEMBRANE PEPTIDE 2
VLIGTSVVKIPFTILLFFLL

SEQ ID NO: 19 - protein, 19 aa, Artificial Sequence
  TRANSMEMBRANE PEPTIDE 3
VLIGTSVVKLPFTILLFFL

SEQ ID NO: 20 - protein, 20 aa, Artificial Sequence
  TRANSMEMBRANE PEPTIDE 4
VLIGTSVVKIPFTILLFFLL

SEQ ID NO: 21 - protein, 19 aa, Artificial Sequence
  TRANSMEMBRANE PEPTIDE 5
VLIGTSVVKLPFTILLFFL

SEQ ID NO: 22 - protein, 20 aa, Artificial Sequence
  TRANSMEMBRANE PEPTIDE 6
ILIGTSVVKIPFTILLFFLL

SEQ ID NO: 23 - protein, 19 aa, Artificial Sequence
  TRANSMEMBRANE PEPTIDE 7
LVPVFCGLLVAKSLSALLV

SEQ ID NO: 24 - protein, 20 aa, Artificial Sequence
  TRANSMEMBRANE PEPTIDE 8
LTAEVLGIICIVLMATVLKT

SEQ ID NO: 25 - protein, 20 aa, Artificial Sequence
  TRANSMEMBRANE PEPTIDE 9
LTAEVLGIICIVLMATVLKT

SEQ ID NO: 26 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 29 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 31 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 32 - DNA, 22 bp, Artificial Sequence
  Primer
gacatgatcgtgaccgataaca

SEQ ID NO: 33 - DNA, 22 bp, Artificial Sequence
  Primer
gcagatagaggtgatggaacaa

SEQ ID NO: 34 - DNA, 24 bp, Artificial Sequence
  Probe
agttctgtgacgtgcggtttagca

SEQ ID NO: 35 - DNA, 22 bp, Artificial Sequence
  Primer
aacaatggcgccgtgaagttcc

SEQ ID NO: 36 - DNA, 22 bp, Artificial Sequence
  Primer
cctcctgtggcttctcgcagat

SEQ ID NO: 37 - DNA, 19 bp, Artificial Sequence
  Primer
ccgccgacaccagactaag

SEQ ID NO: 38 - DNA, 21 bp, Artificial Sequence
  Primer
gctgaacttgtggccgtttac

SEQ ID NO: 39 - DNA, 20 bp, Artificial Sequence
  Probe
tgccatggtgagcaagggcg